(19) United States
(12) Patent Application Publication
(10) Pub. No.: US 2024/0067957 A1
(43) Pub. Date: Feb. 29, 2024

(54) AUTOCATALYTIC BASE EDITING FOR RNA-RESPONSIVE TRANSLATIONAL CONTROL

(71) Applicants: Massachusetts Institute of Technology, Cambridge, MA (US); President and Fellows of Harvard College, Cambridge, MA (US)

(72) Inventors: James J. Collins, Newton, MA (US); Raphael Gayet, Cambridge, MA (US); Katherine IIia, Melrose, MA (US); Shiva Razavi, Cambridge, MA (US); Nathaniel Tippens, Cambridge, MA (US); Kehan Zhang, Brighton, MA (US); Jack Chen, Newton, MA (US); Jonathan Chen, Boston, MA (US); Makoto Lalwani, San Jose, CA (US)

(73) Assignees: Massachusetts Institute of Technology, Cambridge, MA (US); President and Fellows of Harvard College, Cambridge, MA (US)

(21) Appl. No.: 18/339,607

(22) Filed: Jun. 22, 2023

Related U.S. Application Data
(60) Provisional application No. 63/399,845, filed on Aug. 22, 2022.
(60) Provisional application No. 63/481,010, filed on Jan. 23, 2023.

(51) Int. Cl. C12N 15/10; C12N 9/78; C12N 15/11; C12N 15/86

(52) U.S. Cl. CPC C12N 15/1082; C12N 9/78; C12N 15/1086; C12N 15/11; C12N 15/86; C12Y 305/04004; C12N 2310/20; C12N 2310/531; C12N 2750/14143

(57) ABSTRACT

Genetic circuits that control transgene expression in response to pre-defined transcriptional cues would enable the development of smart therapeutics. The present disclosure relates to engineered programmable single-transcript RNA sensors in which adenosine deaminases acting on RNA (ADARs) autocatalytically convert trigger hybridization into a translational output. This system amplifies the signal from editing by endogenous ADAR through a positive feedback loop. Amplification is mediated by the expression of a hyperactive, minimal ADAR variant and its recruitment to the edit site via an orthogonal RNA targeting mechanism. This topology confers high dynamic range, low background, minimal off-target effects, and a small genetic footprint. The circuits and systems disclosed herein leverage an ability to detect single nucleotide polymorphisms and modulate translation in response to endogenous transcript levels in mammalian cells.

### RELATED APPLICATIONS

The application claims the benefit under 35 U.S.C. 119(e) of U.S. Provisional Application No. 63/399,845 filed Aug. 22, 2022, and U.S. Provisional Application No. 63/481,010 filed Jan. 23, 2023, the contents of which are incorporated by reference in their entireties.

### GOVERNMENT SUPPORT

This invention was made with government support under DK120535 and EB024591 awarded by the National Institutes of Health. The government has certain rights in the invention.

### REFERENCE TO AN ELECTRONIC SEQUENCE LISTING

The contents of the electronic sequence listing (M065670526US02-SEQ-JRV.xml; Size: 82,400 bytes; and Date of Creation: Jun. 22, 2023) is herein incorporated by reference in its entirety.

### BACKGROUND

Developing robust tools to modulate the activity of genetic payloads in response to pre-defined cellular cues is a pressing challenge in biomedicine and biological engineering (1). Context-aware genetic circuits would have extensive applications in clinical settings as they could adjust gene expression during disease progression or facilitate precise, cell-specific targeting while minimizing off-target effects (2). Recent advances in transcriptomics have generated rich datasets that capture the molecular signatures of cell states and cell types (3), which could enable efforts to harness this information for the selective, on-demand expression of therapeutic transgenes using novel sense- and-respond modules.

### SUMMARY

RNA-editing technologies have the potential to provide a means to control gene product synthesis to generate therapeutic outcomes. Transcripts of interest (or trigger nucleic acids) can be detected upon specific hybridization with RNA-responsive sensors. As such, a key advantage of RNA as a sensor module is its ability to detect trigger nucleic acids by simple base pairing, thus facilitating the design of highly programmable tools that can be easily repurposed for new applications (4,5). In particular, strand displacement has been explored as a strategy for the direct sensing of trigger nucleic acids both in prokaryotes and eukaryotes (6,7). Recently, the repertoire of transcript-sensing riboregulators was broadened to eukaryotes in a technology known as eToeholds, which relies on engineered mRNA internal ribosome entry sites (IRES) (8). In eToeholds, inhibitory loops of IRES structures are disrupted upon hybridization with target RNAs, thereby restoring ribosome recruitment and enabling RNA-responsive translational control.

Most recently, three groups have independently described convergent approaches to design RNA-based sensors (9-11). In these preliminary reports, base editing by adenosine deaminases acting on RNAs (ADARs) couples the detection of an RNA target to the translation of a user-defined genetic output (such as a payload) (see, FIG. 1A). ADARs efficiently edit mismatched adenosines within imperfect double-stranded RNA (dsRNA) structures (12). The specific hybridization of an engineered sensor transcript with an RNA target of interest therefore allows the conditional recruitment of these RNA-editing enzymes to pre-defined edit sites on the sensors. As adenosines and inosines are interpreted differently by the translational machinery (13), sensor transcript sequences can be designed such that an in-frame UAG stop codon is converted to UIG. As a result of the base editing, the amber codon becomes a sense (tryptophan) codon that does not block translation, leading to the expression of a protein output (such as a payload) encoded downstream of the edited codon (see, FIG. 1A). Through this process, ADAR enzymes convert the detection of an RNA target (via base pairing) into translational activation.

As ADARs are ubiquitous in metazoan cells, these sensors could be used in isolation to detect RNA molecules of interest (see, FIG. 1B, panel i). Although this design paradigm has been validated in vivo in neurons (10), the nervous system is known to express high levels of ADAR (14). Therefore, circuits using endogenous levels of ADARs might not be as effective in other tissues. Overexpression of exogenous ADAR has been explored as a possible solution to enhance the performance of this class of circuits in cells with a lower supply of endogenous ADAR (9,11) (FIG. 1B, panel ii). This, however, results in an increase in the number of transcriptional units, which may hinder the delivery of such a system to cells of interest. Therefore, a circuit topology was designed that overcomes the constraints imposed by the endogenous ADAR supply without stymying delivery.

Accordingly, the present disclosure relates to, in some aspects, a nucleic acid comprising, from 5′ to 3′: (i) a translation initiation sequence; (ii) a sensor sequence comprising a premature stop codon; and (iii) a sequence encoding a base editor that acts on double stranded ribonucleic acid (dsRNA) and a sequence encoding an output, wherein the sequence encoding the base editor and the sequence encoding the output are in frame with the translation imitation sequence. In some embodiments, (i) is separated from (ii) by a sequence encoding a self-cleaving peptide. In some embodiments (ii) is separated from (iii) by a sequence encoding a self-cleaving peptide. In some embodiments, (i) is separated from (ii) and (ii) is separated from (iii) by a sequence encoding a self-cleaving peptide.

In some embodiments, the sequence encoding the base editor and the sequence encoding the output are separated by a sequence encoding a self-cleaving peptide.

In some embodiments, the nucleic acid further comprises a sequence encoding a reporter which is 5′ to the premature stop codon.

In some embodiments, the base editor comprises an adenosine deaminase acting on RNA (ADAR).

In some embodiments, the output sequence comprises a therapeutic protein.

In some embodiments, the therapeutic protein is capable of binding an RNA. In some embodiments, the therapeutic protein comprises a guanine deaminase, a cytidine deaminase, an adenosine deaminase, or a uridine isomerase. In some embodiments, the cytidine deaminase is an apolipoprotein B mRNA editing enzyme catalytic polypeptide (APOBEC).

In some embodiments, the therapeutic protein is capable of processing a gRNA into a mature component of a ribonucleoprotein (RNP).

In some embodiments, the sensor sequence comprises an MS2 hairpin sequence. In some embodiments, the sensor sequence comprises an MS2 hairpin sequence flanking each side of the premature stop codon, and wherein the base editor comprises an MS2 coat protein (MCP).

In some embodiments, the premature stop codon is a TAG stop codon or a UAG stop codon.

In some embodiments, the sensor sequence comprises two or more premature stop codons. In some embodiments, each of the two or more premature stop codons is a TAG stop codon or a UAG stop codon.

In some embodiments, the sensor sequence does not comprise an ATG start codon that: is positioned 3′ to a premature stop codon; and is in frame with the translation initiation sequence. In some embodiments, the sensor sequence does not comprise an ATG start codon that is in frame with the translation initiation sequence.

In some embodiments, the sensor sequence does not comprise a TAG stop codon, a UAG stop codon, a TAA stop codon, a UAA stop codon, a TGA stop codon, or a UGA stop codon.

In some aspects, the present disclosure relates to a vector comprising a nucleic acid sequence encoding a nucleic acid described herein.

In some aspects, the present disclosure relates to a recombinant viral genome comprising a nucleic acid sequence encoding a nucleic acid described herein.

In some embodiments, the recombinant viral genome is a recombinant adeno-associated virus (AAV) genome.

In some aspects, the present disclosure relates to a genetic circuit comprising: an RNA-responsive sensor comprising a nucleic acid as described herein; and a trigger nucleic acid comprising a trigger sequence, wherein the trigger sequence is capable of hybridizing with the sensor sequence of the RNA-responsive sensor, and wherein a nucleotide of the premature stop codon of the sensor sequence is non-complementary to the trigger sequence, and wherein the base editor of the RNA-responsive sensor is capable of editing the nucleotide of the premature stop codon of the sensor sequence that is non-complementary to the trigger sequence.

In some embodiments, the genetic circuit comprises a vector having a nucleic acid sequence encoding the RNA-responsive sensor.

In some embodiments, the genetic circuit comprises a recombinant viral genome having a nucleic acid sequence encoding the RNA-responsive sensor.

In some embodiments, the recombinant viral genome is a recombinant adeno-associated virus (AAV) genome.

In some embodiments, the trigger nucleic acid is a non-coding RNA.

In some embodiments, the trigger nucleic acid is an mRNA.

In some embodiments, the region of the trigger sequence that is capable of hybridizing with the sensor sequence is within the 5′ UTR, the 3′ UTR, or an intron of the mRNA.

In some embodiments, the sensor sequence is designed to encode a TAG stop codon or a UAG stop codon at each position that aligns with a CCA site found in the trigger sequence.

In some embodiments, fewer than 15 nucleic acid mismatches exist between the trigger sequence and the sensor sequence upon their hybridization.

In some embodiments, only one mismatch exists between the trigger sequence and the sensor sequence upon their hybridization.

In some aspects, the present disclosure relates to a cell comprising a genetic circuit described herein, wherein the cell expresses an endogenous base editor that acts on dsRNA.

In some embodiments, the base editor comprises an ADAR.

In some aspects, the present disclosure relates to a method of treating a disease, disorder, or condition in a subject comprising administering to the subject an RNA-responsive sensor comprising a nucleic acid described herein.

In some embodiments, the subject is administered a vector comprising a nucleic acid sequence encoding the RNA-responsive sensor.

In some embodiments, the subject is administered a recombinant viral genome comprising a nucleic acid sequence encoding the RNA-responsive sensor. In some embodiments, the recombinant viral genome is a recombinant adeno-associated virus (AAV) genome.

In some aspects, the present disclosure relates to a method of detecting an RNA molecule in a sample, comprising contacting the sample with a nucleic acids vector described herein, or a recombinant viral genome described herein, wherein the sample comprises a base editor that acts on double stranded RNA or a polynucleotide encoding the same, and mRNA translation machinery; and then detecting output that is produced.

In some embodiments, contacting the sample comprises introducing the nucleic acid, the vector, or the recombinant viral genome into a cell, wherein the cell comprises a base editor or a polynucleotide encoding the same and mRNA translation machinery.

In some aspects, the present disclosure relates to a method of expressing a product of interest in a cell comprising introducing into the cell a nucleic acid described herein, a vector described herein, or a recombinant viral genome described herein, wherein the output of the engineered nucleic acid, the vector, or the recombinant viral genome encodes the product of interest, and wherein the cell expresses a trigger nucleic acid comprising a trigger sequence, wherein the trigger sequence is capable of hybridizing with the sensor sequence of the nucleic acid, and wherein a nucleotide of the premature stop codon of the sensor sequence is non-complementary to the trigger sequence, and an endogenous base editor that is capable of editing the nucleotide of the premature stop codon of the sensor sequence that is non-complementary to the trigger sequence. In some embodiments, the base editor comprises an ADAR.

### BRIEF DESCRIPTION OF DRAWINGS

FIGS. 1A-1F show non-limiting examples of designs for adenosine deaminase acting on RNA (ADAR)-mediated RNA-responsive sensors and testing of the same. FIG. 1A shows a schematic presenting an overview of a basic ADAR-mediated RNA-responsive sensor. In their inactive state, ADAR-mediated RNA-responsive sensors include a premature stop codon (UAG), which precludes translation of a downstream output. ADAR-mediated RNA-responsive sensors are activated by the specific hybridization of trigger nucleic acids, followed by the enzymatic deamination of the mismatched A in the premature stop codon. In this way the premature stop codon is converted to UIG, which is read as UGG (a codon for tryptophan), and translation of the output is activated. FIG. 1B shows schematics presenting three additional ADAR-mediated RNA-responsive sensor designs. Pros (in green) and cons (in orange) inherent in these designs are summarized on the right-hand side of panels i, ii, and iii. (i) ADAR-mediated RNA-responsive sensors can be designed such that there is no exogenous supplementation of ADAR. In such systems, the low levels of endogenous ADAR carry out editing of a subset of sensor molecules. (ii) Other embodiments include constitutive overexpression of exogenous ADAR. Here, high levels of ADAR efficiently mediate editing of sensor sequences, but these systems are more difficult to deliver, and exogenous ADAR is overexpressed in all cells, resulting in unnecessary consumption of cellular resources. (iii) An embodiment that builds on the advantages of the aforementioned approaches is based on conditional expression of exogenous ADAR. In such circuits, endogenous ADAR mediates editing in a small subpopulation of sensor molecules. This step prompts the translation of the circuit payload, which includes ADAR itself. After this initial editing step by endogenous ADAR, the system produces ADAR in order to increase the frequency of editing events and achieve higher dynamic range as a result of this amplification step. m7G: mRNA cap; 2A: self-cleaving 2A peptide; AAA: poly(A) tail. FIG. 1C shows exogenous supplementation of ADAR improved sensor performance. Numbers following the CCAs indicate the nucleotide position of the premature stop codon, using the start codon as position+1. Output fold-change (FC) is the ratio of the geometric mean of mNeonGreen expression in the presence and absence of trigger nucleic acid. Error bars represent 95% confidence intervals for the fold-change values. FIG. 1D shows fluorescence microscopy of mNeonGreen illustrating the performance of the CCA60 sensor against iRFP720 in HEK293FT cells, 48 hr after transfection (Scale bar: 300 μm). FIG. 1E shows sequencing data confirmed increased A-to-I editing of the CCA60 sensor in the presence of trigger nucleic acid in samples where exogenous ADAR p150 is supplied. The sequence logo demonstrated ADAR-mediated editing is specific to the central A in the UAG stop codon. Data collected on n=3 biological replicates. FIG. 1F shows the system relied on an initial editing step by endogenous ADAR enzymes, which was then amplified by the translation of additional ADAR (black bars indicate positions within the ADAR-mediated RNA-responsive sensor where self-cleaving peptide sequences are located).

FIGS. 2A-2E show optimization of non-limiting examples of ADAR-mediated RNA-responsive sensors. FIG. 2A shows a schematic depicting RNA-responsive sensors and a corresponding trigger nucleic acid having a region of complementarity (i.e., trigger sequence) with the sensor in its coding sequence. It was hypothesized that the RNA-responsive sensors would work best if the premature stop codon did not hybridize to a region of the trigger nucleic acid that may be bound by ribosomes (orange). As such, the performance of RNA-responsive sensors designed against secreted proteins or 3′UTR sequences was expected to be enhanced, as stalled and dissociated ribosomes are less likely to disrupt sensor-trigger nucleic acid hybridization. SP: signal peptide; UTR: untranslated region. FIG. 2B shows ADAR-mediated RNA-responsive sensors yielded higher dynamic range when designed to hybridize with the 3′UTR of a trigger nucleic acid or the coding sequences of a secreted protein. “allstop” indicates that all the sites in the sensor sequence aligning with CCA sites in the trigger nucleic acid were made into editable UAG codons (as opposed to only the central codon). Fold-change (FC) is the ratio of the geometric mean of mNeonGreen expression in the presence and absence of trigger nucleic acid. Error bars represent 95% confidence intervals for the fold-change values. FIG. 2C shows MCP-ADAR2dd is a compact base editor that acts on double-stranded RNA, and the MCP-MS2 binding system facilitated the specific recruitment of the enzyme to the sensor sequence. NES: nuclear export sequence; ZBDs: Z-DNA binding domains; dsRBDs: dsRNA-binding domains; NLS: nuclear localization signal; MCP: MS2 major coat protein; m7G: mRNA cap; 2A: self-cleaving 2A peptide; AAA: poly(A) tail. FIG. 2D shows the functionality of sensors containing MS2 hairpins without ADAR, with ADAR p150, or with MCPADAR2dd. The OFF and ON states corresponded to mNeonGreen expression in the absence and presence of iRFP720 trigger nucleic acid mRNA, respectively. FIG. 2E shows MCP-ADAR specifically activated the translation of payloads encoded in ADAR-mediated RNA-responsive sensors containing MS2 RNA hairpins.

FIGS. 3A-3K show autocatalytic ADAR-mediated RNA-responsive sensors are responsive, specific, and sensitive. FIG. 3A shows a schematic illustrating the ADAR-mediated RNA-responsive sensor design. FIG. 3B shows ADAR-mediated RNA-responsive sensors modified by adding two flanking MS2 hairpins upstream and downstream of the edit site in three configurations. Numeric annotations correspond to the number of base pairs between the edit site and MS2 hairpin. L, left; R, right; C, center. FIG. 3C shows a positive feedback loop, the system disclosed herein is a closed-loop (CL) system. To benchmark the system's performance, an open-loop (OL) control was constructed in which the ADAR was constitutively expressed and supplied in trans. FIG. 3D shows the fold-change (FC) of the geometric mean of mNeonGreen (output). Expression levels are plotted for open-loop (OL) and closed-loop (CL) variants. ADAR-mediated RNA-responsive sensors represented by points that fall near the blue dashed diagonal line were minimally improved by autocatalysis, whereas points that fall above this line corresponded to ADAR-mediated RNA-responsive sensors that perform better with autocatalysis. FIG. 3E shows a comparison of the ratio of basal activity (x-axis) and fold-change (y-axis) in open- and closed-loop ADAR-mediated RNA-responsive sensor variants demonstrated the performance boost provided by the autocatalytic architecture of the system. Closed-loop ADAR-mediated RNA-responsive sensors with x-axis values below zero demonstrated a decrease in mNeonGreen expression in the absence of trigger nucleic acid and closed-loop ADAR-mediated RNA-responsive sensors with y-axis values above zero showed an increase in dynamic range. FIG. 3F shows open- and closed-loop ADAR-mediated RNA-responsive sensors have different transfer curves for a given sensor expression level. The iRFP720 bin #1 corresponds to a “no-trigger” (i.e., no trigger nucleic acid) condition. For each variant, mNeonGreen expression is normalized to the maximal sensor activation (bin #7). FIG. 3G shows ADAR-mediated RNA-responsive sensors can be designed to specifically activate in response to a point mutation (n=3 biological replicates). WT: wild-type p53 mRNA; Y220H: mutant p53 mRNA. CL: closed-loop sensors; OL: open-loop version of CL, without constitutive ADAR supplementation; OL+MCP-ADAR: open-loop version of CL, with constitutive MCP-ADAR supplementation.

FIG. 3H shows C2C12 cell differentiation steered towards the myoblastic or the osteoblastic lineage. Top right: Hoechst 33342 (DNA, blue) and CFSE (whole cells, green) staining demonstrated the presence of multinucleated syncytia (arrows) two days after switching C2C12 cells to the differentiation-inducing medium. Bottom right: a colorimetric BCIP/NBT assay detects alkaline phosphatase activity in C2C12 cells treated with BMP-2 for 8 days.

FIG. 3I shows RT-qPCR gene expression analysis highlighted muscle and bone lineage-specific markers in undifferentiated and differentiated C2C12 cells. Bars represent mean and standard deviation measured on n=3 technical replicates. FIG. 3J shows ADAR-mediated RNA-responsive sensors targeting endogenous myosin heavy chain (Myh7) and myogenin (Myog) mRNAs were specifically activated between days 0 and 2 post-induction of differentiation. Backbone: premature stop codon absent from sensor sequence, constitutively active; N1, N2: ADAR-mediated RNA-responsive sensors for osteoblastic differentiation (negative controls). K. Three ADAR-mediated RNA-responsive sensors targeting endogenous alkaline phosphatase (ALP) mRNA were detected on days 0 and 8 post-treatment with BMP-2. Backbone: premature stop codon absent from sensor sequence, constitutively active; N1, N2: ADAR-mediated RNA-responsive sensors for myogenic differentiation (negative controls).

FIG. 4 shows an exemplary RNA-responsive sensor having a TagBFP reporter sequence was included at the 5′ end of the sensor transcript to account for plasmid dosage, and an mNeonGreen coding sequence was included downstream of the sensor sequence as the output. All the elements are insulated by self-cleaving 2A peptide sequences. “m7G” corresponds to mRNA cap; “2A” corresponds to self-cleaving 2A peptide; “AAA” corresponds to poly(A) tail.

FIGS. 5A-5C show flow cytometry analysis of the experimental pipeline. FIG. 5A shows a gating strategy wherein cells are gated on forward- and side-scatter signals. FIG. 5B shows a binning strategy wherein flow cytometry data was binned at half-log intervals, excluding datapoints with saturated fluorescence measurements. Representative data for CCA60 in the absence (left plot) or presence (right plot) of secreted iRFP720 trigger is shown. FIG. 5C shows an overview of the workflow used for data processing.

FIGS. 6A-6B show the performance of non-limiting examples of shorter RNA-responsive sensors. FIG. 6A shows the exogenous supplementation of ADAR improved the performance of 51 bp sensors, although these had slightly lower dynamic range compared to the 75 bp sensors. Fold-change is the ratio of the geometric mean of mNeonGreen expression in the presence and absence of trigger. Error bars represent 95% confidence intervals for the fold-change values. FIG. 6B shows 75 bp and 51 bp ADAR-mediated RNA-responsive sensors also yielded higher dynamic range when targeting 3′UTRs or transcripts of secreted proteins. Fold-change is the ratio of the geometric mean of mNeonGreen expression in the presence and absence of trigger. Error bars represent 95% confidence intervals for the fold change values.

FIGS. 7A-7B show the performance of non-limiting examples of ADAR-mediated RNA-responsive sensors targeting coding sequences of cytosolic proteins, secreted proteins, and 3′ UTRs. ADAR-mediated RNA-responsive sensors yielded higher dynamic range when designed to target the 3′UTRs of transcripts (as is the case for NanoLuc luciferase in FIG. 7A), or coding sequences of secreted proteins (as is the case for puromycin acetyltransferase (PAC) in FIG. 7B). The fold change (FC) is the ratio of the geometric means of mNeonGreen expression in the presence and absence of trigger. Error bars represent 95% confidence intervals for the fold-change values.

FIG. 8 shows nuclear transcript detection data. ADAR-mediated RNA-responsive sensors targeting the nuclear lncRNA MALAT1, as well as exogenous ADAR p150 and p110, were transiently transfected in two A549 cell lines. “WT cells” corresponds to parental cell line; “A cells” corresponds to MALAT1 knock-out derivative. The sensor output expression was comparable in both cell lines across 16 different sensors, suggesting that current ADAR-mediated RNA-responsive sensors function in the cytosol.

FIGS. 9A-9B show changes in translational outputs related to changes in A:I RNA editing in HEF293T cells transfected with different sensors and corresponding trigger nucleic acids. FIG. 9A shows flow cytometry analysis of output protein expression levels. FIG. 9B shows next generation sequencing analysis of the frequencies of A:I editing in the sensor UAG stop codon. Bars represent the means of n=3 biological replicates. “CL” corresponds to closed loop (DART VADAR); “OL” corresponds to open loop; “AFU” corresponds to arbitrary fluorescent units.

FIGS. 10A-10C show the effect of sensor length in the absence of exogenous ADAR. FIG. 10A shows the performance of ADAR-mediated RNA-responsive sensors relying only on endogenous ADAR was not appreciably improved with increased sensor length. “FC” corresponds to fold change. FIGS. 10B-10C show RT-qPCR gene expression analyses of the expression of transcripts involved in the dsRNA immune response as a function of the length of the sensor-trigger duplexes. FIG. 10B shows RT-qPCR analysis of IFNB1. FIG. 10C shows RT-qPCR analysis of IFH1. Fold changes represent the ratios of GAPDH-normalized transcript abundances in the presence and absence of RNA trigger. Bars represent the means of n=3 biological replicates. “CL” corresponds to closed loop (DART VADAR); “OL” corresponds to open loop.

FIG. 11 shows analysis of ADAR-mediated global off-target effects.

FIG. 12 shows an non-limiting example of a step-by-step guide for designing RNA-responsive sensors comprising MS2 hairpins.

### DETAILED DESCRIPTION

Described herein are engineered nucleic acids encoding RNA-responsive sensors and genetic circuits comprising the same. These RNA-responsive sensors allow one to couple the detection of a trigger nucleic acid to a molecular response (e.g., translation of an output encoded by an RNA-responsive sensor). Also described herein are compositions and kits comprising an RNA-responsive sensor (or an engineered nucleic acid encoding the same), as well as various methods, such as methods of treatment, methods of detecting an RNA molecule in a sample, and methods expressing a product of interest.

### I. RNA-Responsive Sensors

In some aspects, the disclosure relates to RNA-responsive sensors. In some embodiments, the RNA-responsive sensors described herein are single stranded RNA (ssRNA) molecules that comprise, from 5′ to 3′: (i) a translation initiation sequence; (ii) a sensor sequence that comprises a premature stop codon; and (iii) a sequence encoding a base editor that acts on double-stranded RNA (dsRNA) (such as an ADAR protein) and a sequence encoding an output.

In some embodiments, the sequence encoding the base editor that acts on dsRNA is 5′ to the sequence encoding the output sequence. In other embodiments, the sequence encoding the base editor is 3′ to the sequence encoding the output sequence. In some embodiments, the sequence encoding the base editor and the sequence encoding the output are separated by a sequence encoding a self-cleaving peptide.

In some embodiments, (i) is separated from (ii) by a sequence encoding a self-cleaving peptide. In some embodiments, (ii) is separated from (iii) by a sequence encoding a self-cleaving peptide. In some embodiments, (i) is separated from (ii) by a sequence encoding a self-cleaving peptide, and (ii) is separated from (iii) by a sequence encoding a self-cleaving peptide.

In some embodiments, an RNA-responsive sensor comprises a sequence encoding a reporter. In some embodiments, the sequence encoding the reporter is 5′ to the sensor sequence. In some embodiments, the sequence encoding the reporter is positioned between the translation initiation sequence and the sensor sequence.

A. Translation Initiation Sequence

In some embodiments, the RNA-responsive sensors described herein comprise a translation initiation sequence. As used herein, a “translation initiation sequence” refers to a sequence that is capable of being bound by a ribosome such that the ribosome can initiate translation in the presence of other translational machinery. Exemplary translation initiation sequences (for various expression systems) are known to those having ordinary skill in the art.

In some embodiments, a translation initiation sequence is a cap-dependent translation initiation site. In some embodiments, a cap-dependent translation initiation site comprises the sequence GCCRCSATGN (SEQ ID NO: 66), wherein “r” is a purine (a/g), “s” is a strong base (c/g), “n” is any base, and the capital “ATG” is the initiation methionine. In some embodiments, a cap dependent translation initiation site comprises the sequence GCCACCATGA (SEQ ID NO: 67).

In some embodiments, a translation initiation sequence is a cap-independent translation initiation site, such as a translation initiation sequence comprising an internal ribosome entry site (IRES). Exemplary IRES sequences are known to those having ordinary skill in the art. In some embodiments, an IRES comprises those derived from viruses, such as encephalomyocarditis virus (EMCV), coxsackievirus B3 (CVB3), polioviruses, cricket paralysis virus (CrPV), enterovirus 71 (EV71), foot-and-mouth disease virus (FMDV), hepatitis A virus (HAV), and hepatitis C virus (HCV). In some embodiments, an IRES comprises those from eukaryotic sequences, such as c-myc or vascular endothelial growth factor (VEGF).

In some embodiments, a cap-independent translation initiation sequence comprises a 5′ UTR comprising N⁶-methyladenosine (m⁶A). See, e.g., Meyer et al., (2015). 5′ UTR m⁶A Promoters Cap-Independent Translation. Cell, 163(4): 999-1010.

B. Sensor Sequence

In some embodiments, the ADAR-mediated RNA-responsive sensors described herein comprise a sensor sequence. As used herein, a “sensor sequence” refers to a nucleic acid sequence that is designed to and is capable of hybridizing with a portion of a trigger nucleic acid (an RNA molecule which is described in Part III below), for example under the physiological conditions of a cell.

In some embodiments, the sensors of the RNA-responsive sensors described herein comprise a premature stop codon. In some embodiments, this premature stop codon prevents translation of sequences 3′ to the sensor sequence (e.g., the sequence encoding the base editor that acts on double-stranded RNA and the sequence encoding the output). However, in some embodiments, mutation of the premature stop codon (e.g., by deamination mediated by a base-editor that acts on dsRNA), such that it no longer is a premature stop codon, enables the translation of the sequence 3′ of the sensor sequence, when in the presence of translational machinery.

In some embodiments, a sensor comprises a TAG stop codon or a UAG stop codon. In some embodiment, a sensor comprises a TAA stop codon or a UAA stop codon. In some embodiments, a sensor comprises a TGA stop codon or a UGA stop codon.

In some embodiments, a sensor comprises more than one premature stop codon. In some embodiments, a sensor comprises 2, 3, 4, 5, 6, 7, 8, 9, or more premature stop codons.

In some embodiments, a sensor is designed to encode a UAG stop codon at each position that aligns with a CCA site found in a trigger sequence (described below).

In some embodiments, the sensor sequence does not comprise an ATG start codon that: is positioned 3′ to a premature stop codon; and is in frame with the translation initiation sequence. In some embodiments, the sensor sequence does not comprise an ATG start codon that is in frame with the translation initiation sequence.

In some embodiments, the sensor sequence does not comprise a TAG stop codon, a UAG stop codon, a TAA stop codon, a UAA stop codon, a TGA stop codon, or a UGA stop codon.

Sensor sequences vary in length. In some embodiments, a sensor sequence is at least 10, at least 15, at least 20, at least 25, at least 30, at least 35, at least 40, at least 50, at least 60, at least 70, at least 80, at least 90, at least 100, at least 120, at least 140, at least 160, at least 180, or at least 200 nucleotides in length. In some embodiments, a sensor sequence is no more than 200, no more than 180, no more than 160, no more than 140, no more than 120, no more than 100, no more than 90, no more than 80, no more than 70, no more than 60, no more than 50, no more than 40, no more than 25, or no more than 20 nucleotides in length. In some embodiments, a sensor sequence is 10-15, 15-20, 20-25, 25-30, 30-35, 35-40, 40-45, 45-50, 50-55, 55-60, 60-65, 65-70, 70-75, 75-80, 80-85, 85-90, 90-95, 95-100, 100-110, 110-120, 120-130, 130-140, 140-150, 150-160, 160-170, 170-180, 180-190, 190-200, 200-225, 225-250, 250-275, or 275-300 nucleotides in length. In some embodiments, a sensor sequence comprises about 50 or 75 nucleotides in length.

In some embodiments, a sensor sequence comprises a functional nucleic acid motif that is capable of recruiting a factor(s) to the sensor sequence (i.e., is capable of being bound by such factor(s)).

In some embodiments, at least one functional nucleic acid motif is located at the most 5′ or 3′ end of a sensor sequence. In some embodiments, a functional nucleic acid motif is separated from the premature stop codon of a sensor sequence by at least 20, at least 21, at least 22, at least 23, at least 24, at least 25, at least 26, at least 27, at least 28, at least 29, or at least 30 nucleotides. In some embodiments, a functional nucleic acid motif is separated from the premature stop codon by no more than 20, no more than 21, no more than 22, no more than 23, no more than 24, no more than 25, no more than 26, no more than 27, no more than 28, no more than 29, or no more than 30 nucleotides. In some embodiments, a functional nucleic acid motif is separated from the premature stop codon by 20-30, 21-30, 22-30, 23-30, 24-30, 25-30, 26-30, 27-30, 28-30, 29-30, 20-25, 21-25, 22-25, 23-25, or 24-25 nucleotides. In some embodiments, a functional nucleic acid motif is located within 20, 21, 22, 23, 24, 25, 26, 27, 28, 29, or 30, or more nucleotides upstream and/or downstream of the premature stop codon of the sensor sequence.

In some embodiments, a sensor sequence comprises 1, 2, 3, 4, 5, 6, 7, 8, 9, 10, or more functional nucleic acid motifs. In some embodiments a sensor sequence comprises a functional nucleic acid motif(s) flanking the premature stop codon on the 5′ end, and a functional nucleic acid motif(s) flanking the premature stop codon on the 3′ end. In some embodiments, a functional nucleic acid motif flanking the premature stop codon on the 5′ end and a functional nucleic acid motif flanking the premature stop codon on the 3′ end are identical (i.e., comprise the same sequence). In some embodiments, a functional nucleic acid motif flanking the premature stop codon on the 5′ end and a functional nucleic acid motif flanking the premature stop codon on the 3′ end are different (i.e., comprise different sequences).

In some embodiments, a functional nucleic acid motif promotes recruitment of endogenous proteins of a cell to the RNA-responsive sensor. In some embodiments, a functional nucleic acid motif promotes recruitment of exogenous proteins of a cell to the RNA-responsive sensor. In some embodiments, a functional nucleic acid motif promotes recruitment of both endogenous and exogenous proteins of a cell to the RNA-responsive sensor. In some embodiments, a functional nucleic acid motif promotes recruitment of a base editor that act on dsRNA to the RNA-responsive sensor.

In some embodiments, a sensor sequence comprises a functional nucleic acid motif that folds or base pairs intramolecularly to assume a conformation recognized by a protein. In some embodiments, the sensor sequence may feature one or more hairpin sequences.

In some embodiments, a functional nucleic acid motif comprises an MS2 hairpin. Exemplary sequences encoding MS2 RNA hairpins are known to those having ordinary skill in the art. In some embodiments, an MS2 RNA hairpin comprises a nucleic acid sequence having at least 80%, at least 85%, at least 90%, at least 92%, at least 95%, at least 98%, or at least 99% identity with the nucleic acid sequence of SEQ ID NO: 1 or 2. In some embodiments, an MS2 RNA hairpin comprises or consists of the nucleic acid sequence of SEQ ID NO: 1 or 2.

In some embodiments, a MS2 RNA hairpin flanks the 5′ end of the premature stop codon. In some embodiments, a MS2 RNA hairpin flanks the 3′ end of the premature stop codon. In some embodiments, a MS2 RNA hairpin flanks the 5′ end of the premature stop codon, and a MS2 RNA hairpin flanks the 3′ end of the premature stop codon.

In some embodiments, a sensor sequence comprises a hairpin that binds to a protein derived from Pseudomonas bacteriophage PP7, a protein derived from bacteriophage Qβ, λN22 protein, a protein that binds to boxB RNA, an L7Ae protein having a k-turn motif, a pumilio homology domain (PUM-HD), or a tetR-DDX6 fusion protein. In some embodiments, a sensor sequence comprises an operator hairpin sequence from the genome of a Fiersviridae virus family member.

In some embodiments, a sensor sequence may comprise an engineered nucleic acid sequence (e.g., an engineered sequence that forms a hairpin) that is engineered to recruit proteins to facilitate an RNA-protein interaction.

In some embodiments, a sensor sequence comprises an aptamer.

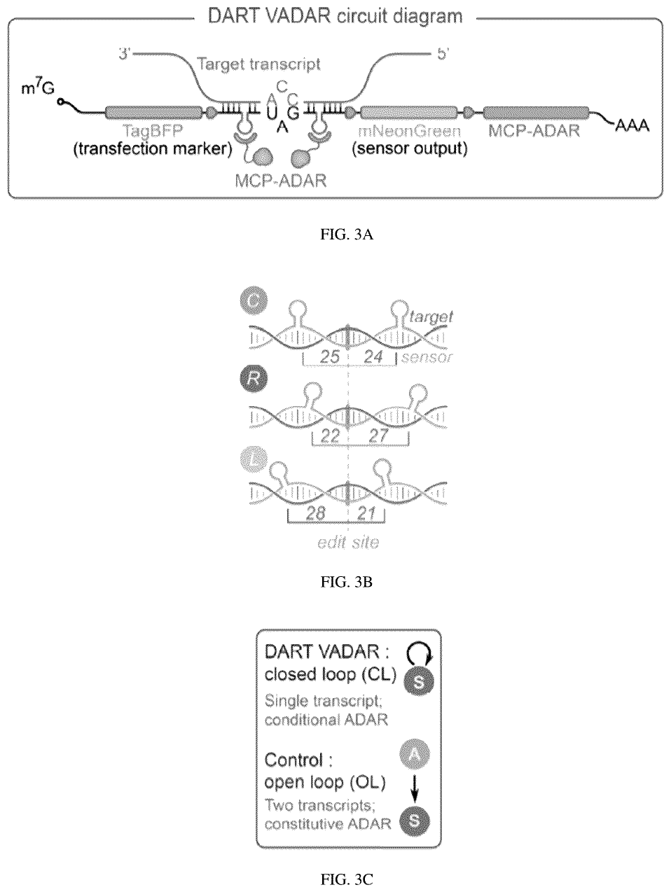

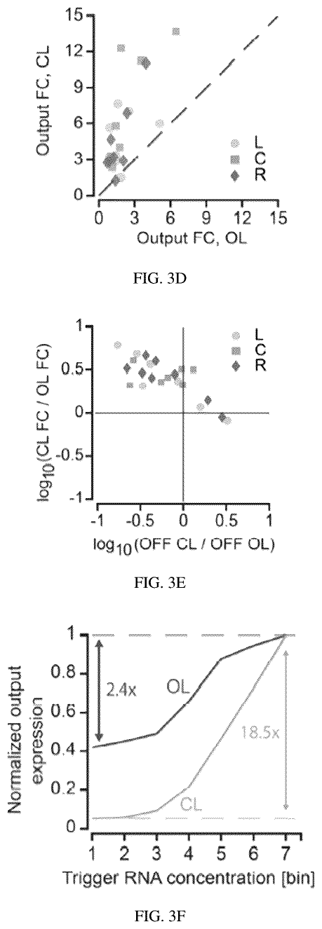

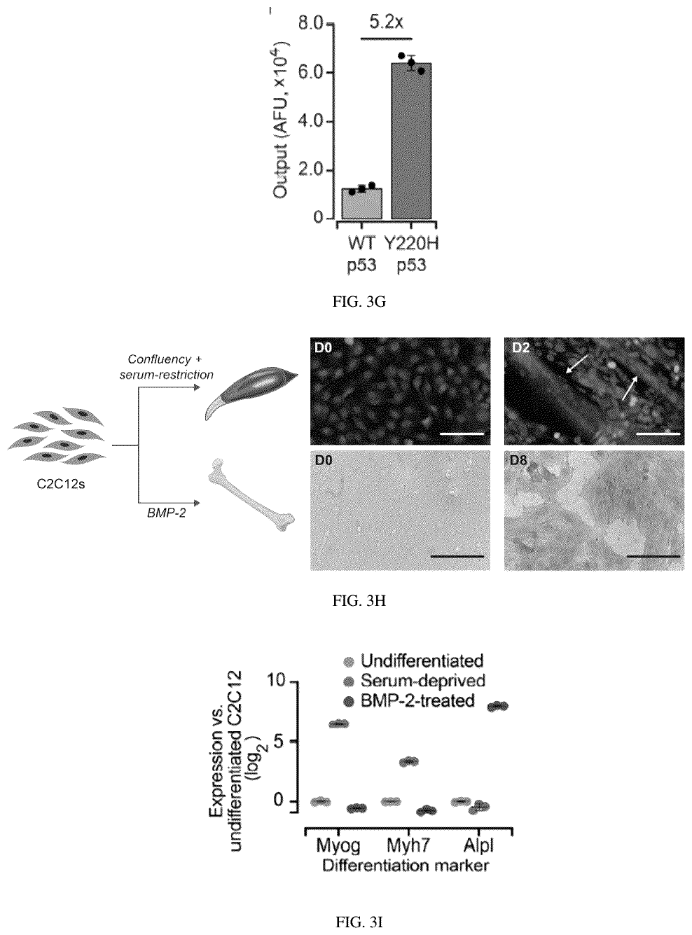

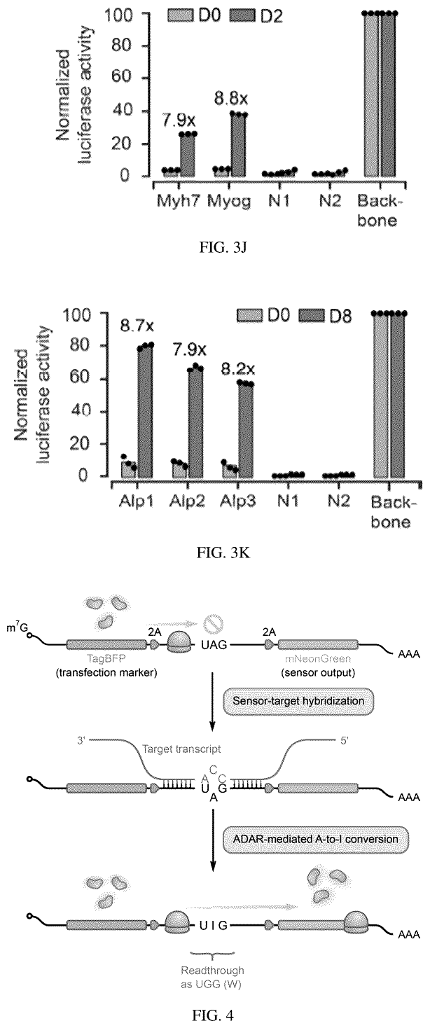

In some embodiments, a sensor sequence may be designed to detect sequence-specific variants of an mRNA transcript. In some embodiments, a sensor sequence may be designed to detect sequence-specific variants comprising a single nucleotide polymorphism (see, e.g., FIG. 3G). In some embodiments, a sensor sequence may be designed to detect sequence-specific variants comprising a plurality of mutations (e.g., substitutions, insertions, and/or deletions). In some embodiments, a sensor sequence may be designed to detect sequence-specific variants comprising a splice variant.

In some embodiments, the RNA-responsive sensors described herein comprise a nucleotide sequence having at least 80%, at least 85%, at least 90%, at least 92%, at least 95%, at least 98%, or at least 99% identity to any one of SEQ ID NOs: 21-47. In some embodiments, the RNA-responsive sensors described herein comprise the nucleotide sequence of any one of SEQ ID NOs: 1-2 or 21-47.

C. Base Editor that Acts on Double-Stranded RNA

In some embodiments, the RNA-responsive sensors described herein comprise a sequence encoding a base editor that acts on dsRNA. As used herein, a “base editor that acts on dsRNA” is an enzyme capable of binding to a double-stranded RNA molecule and editing a nucleotide of the double-stranded molecule (e.g., a nucleotide of a premature stop codon), for examples via deamination.

In some embodiments, a base editor that acts on dsRNA comprises an adenosine deaminase acting on RNA (ADAR) protein. Exemplary ADAR proteins are known to those have ordinary skill in the art. In mammals, there are three types of ADAR proteins: ADAR (ADAR1); ADARB1 (ADAR2); and ADARB2 (ADAR3). ADAR and ADARB1 are found in many tissues in the mammalian body while ADARB2 is only found in the brain.

In some embodiments, the base editor that acts on dsRNA comprises ADAR1, or a functional variant thereof. In some embodiments, the base editor that acts on dsRNA comprises an ADAR1 isoform such as p110 or p150.

In some embodiments, the base editor that acts on dsRNA comprises an ADAR described herein, or a functional variant thereof. In some embodiments, the ADAR is a minimal variant of ADAR. In some embodiments, a minimal variant of ADAR comprises an engineered amino acid sequence lacking one or more domains, or a portion thereof, that are naturally found in ADAR. In some embodiments, a minimal variant of ADAR comprises an amino acid sequence that changes the RNA-binding properties of ADAR. In some embodiments, a minimal variant of ADAR comprises one or more bacteriophage major coat protein (MCP) sequences that specifically bind to short MS2 RNA hairpin which replaces the dsRNA-interacting domains of natural ADAR enzymes

In some embodiments, the base editor that acts on dsRNA comprises ADAR2, or a functional variant thereof. In some embodiments, the ADAR2 is a minimal variant of ADAR2. In some embodiments, a minimal variant of ADAR2 comprises an engineered amino acid sequence lacking one or more domains, or a portion thereof, that are naturally found in ADAR2. In some embodiments, a minimal variant of ADAR2 comprises an amino acid sequence that changes the RNA-binding properties of ADAR2. In some embodiments, a minimal variant of ADAR2 comprises one or more bacteriophage major coat protein (MCP) sequences that specifically bind to short MS2 RNA hairpin which replaces the dsRNA-interacting domains of natural ADAR enzymes

In some embodiments, the base editor that acts on dsRNA comprises ADAR3, or a functional variant thereof.

In some embodiments, an ADAR protein of a base editor that acts on dsRNA described herein comprises an amino acid sequence having at least 80%, at least 85%, at least 90%, at least 92%, at least 95%, at least 98%, or at least 99% identity to any one of SEQ ID NOs: 3-6 or 9. In some embodiments, an ADAR protein comprises or consists of the amino acid sequence of any one of SEQ ID NOs: 3-6 or 9. In some embodiments, an ADAR protein of a base editor that acts on dsRNA described herein comprises an amino acid sequence having at least 80%, at least 85%, at least 90%, at least 92%, at least 95%, at least 98%, or at least 99% identity to any one of SEQ ID NOs: 3-6 or 9 and comprises one or more mutations that increases the catalytic activity of the ADAR (e.g., a variant comprising the E488Q substitution). In some embodiments, an ADAR protein of a base editor that acts on dsRNA described herein comprises an amino acid sequence having at least 80%, at least 85%, at least 90%, at least 92%, at least 95%, at least 98%, or at least 99% identity to any one of SEQ ID NOs: 3-6 or 9 and further comprises one or more additional polypeptides that are operably linked thereto. In some embodiments, an ADAR protein comprises or consists of the amino acid sequence of any one of SEQ ID NOs: 3-6 or 9 and further comprises one or more additional polypeptides that are operably linked thereto. In some embodiments, the one or more additional polypeptides that are operably linked comprises a reporter (e.g., a fluorescent protein, such as GFP).

In some embodiments, a base editor that acts on dsRNA is a fusion protein that further comprises a MS2 bacteriophage major coat protein (MCP) which specifically binds to a short MS2 RNA hairpin. Exemplary MCP proteins are known to those having ordinary skill in the art. In some embodiments, an MCP protein comprises an amino acid sequence having at least 80%, at least 85%, at least 90%, at least 92%, at least 95%, at least 98%, or at least 99% identity with the amino acid sequence of SEQ ID NO: 7. In some embodiments, an MCP protein comprises or consists of the amino acid sequence of SEQ ID NO: 7. In some embodiments, a fusion protein comprises a base editor that is directly linked to an MCP. In some embodiments, a fusion protein comprises a base editor that is indirectly linked to an MCP. For example, in some embodiments, the fusion protein further comprises one or more peptide linkers that separate the base editor from the MCP. Exemplary peptide linkers are known to those having ordinary skill in the art, such as Gly-Ser linkers.

In some embodiments, a base editor that acts on dsRNA further comprises a nuclear export signal (NES). In some embodiments, a base editor that acts on dsRNA further comprises a nuclear localization sequence (NLS). In some embodiments, the base editor that acts on dsRNA comprises both an NES and an NLS. Exemplary nuclear export signals and nuclear localization signals are known to those having ordinary skill in the art.

In some embodiments, the base editor that acts on dsRNA comprises the structure MCP-ADAR protein-NES. In some embodiments, the base editor that acts on dsRNA comprises the structure MCP-ADAR2D-NES. In some embodiments, the base editor comprises an amino acid sequence having at least 80%, at least 85%, at least 90%, at least 92%, at least 95%, at least 98%, or at least 99% identity to SEQ ID NO: 8. In some embodiments, the base editor comprises or consists of the amino acid sequence of SEQ ID NO: 9. In some embodiments, the base editor that acts on dsRNA comprises the structure MCP-ADAR2DD(E488Q)-NES.

D. Output

In some embodiments, the RNA-responsive sensors described herein comprise a sequence encoding an output. In some embodiments, the output of an RNA-responsive sensor encodes a peptide or polypeptide.

In some embodiments, an output (when translated) is capable of altering the physiological state of a cell. In some embodiments, altering the physiological state of a cell includes, but is not limited to, regulating a metabolic pathway, regulating the expression of a gene product(s), and regulating a signaling pathway in a cell. In some embodiments, the output comprises a wild-type form of a peptide or a polypeptide that is either not expressed or expressed at low levels as the result of a disease or a disorder. In some embodiments, the output comprises a wild-type form of a peptide or protein thereby compensating for the expression of a mutant or variant form of the protein expressed in a cell (e.g., a cell of a subject) as the result of a disorder or disease. In some embodiments, the output comprises an enzyme that regulates a pathway that is complementary or antagonistic to a protein that is mutated in a cell thereby compensating for the mutated protein. In some embodiments, an output comprises an enzyme that regulates the degradation of another protein, such as a mutated protein. In some embodiments, an output comprises a protein that makes a cell responsive to a drug such as, but not limited to, a cell surface protein that can be targeted by an agent that binds to an antigen. In some embodiments, an output comprises an allosteric modulator of an enzyme or an enzyme that produces an allosteric modulator.

In some embodiments, the output comprises a component of a gene editing system. In some embodiments, the output comprises a nuclease such as a TALEN or a zinc-finger nuclease. In some embodiments, the output comprises a CRISPR-effector protein such as a Cas molecule, a Cpf1 molecule, a base editor, or a primer editor. In some embodiments, a Cas molecule includes, but is not limited to, Streptococcus pyogenes Cas9 (spCas9), Staphylococcus aureus Cas9 (saCas9), Cas12a, Cas12b, Cas13, nicking Cas9 (nCas9), or dead Cas9 (dCas9). In some embodiments, the Cpf1 molecule includes, but is not limited to, AsCas12a, FnCas12a, LbCas12a, PaCas12a, other Cpf1 orthologs, and Cas12a derivatives, such as the MAD7 system (MAD7™, Inscripta, Inc.), or the Alt-R Cas12a (Cpf1) Ultra nuclease (Alt-R® Cas12a Ultra; Integrated DNA Technologies, Inc.). In some embodiments, the Cpf1 domain is from Cas12a/Cpf1 obtained from Acidaminococcus sp. (referred to as “AsCas12a” or “AsCpf1”), such as Acidaminococcus sp. Strain BV3L6. In some embodiments, the output comprises a guanine deaminase. In some embodiments, the output comprises a cytidine deaminase. In some embodiments, the output comprises a uridine isomerase. In some embodiments, the output comprises an apolipoprotein B mRNA editing enzyme, catalytic polypeptide (APOBEC). APOBECs catalyzes C:U conversion using a catalytic domain comprising a cytidine deaminase. Examples of APOBEC family members include APOBEC1, APOBEC2, APOBEC3A, APOBEC3B, APOBEC3C, APOBEC3D, APOBEC3E, APOBEC3F, APOBEC3H, APOBEC3H, APOBEC4, and activation-induced (cytidine) deaminase (AID). In some embodiments, the output comprises an adenosine deaminase such as ADA1 or ADA2.

In some embodiments, the output comprises a protein that regulates the maturation of a component of a ribonucleoprotein protein (RNP) complex such as a nucleic acid programmable nuclease or a guide RNA. In some embodiments, the output (when translated) can process a gRNA into a mature component of an RNP. In some embodiments, the output comprises a protein that regulates the transcription or modification of a nucleic acid programmable nuclease or a guide RNA thereby regulating the maturation of a ribonucleoprotein complex.

In some embodiments, the output (when translated) is capable of binding RNA. In some embodiments, the output (when translated) can mutate RNA (i.e., induce one or more nucleotide changes).

In some embodiments, the output sequence may comprise a detectable protein.

In some embodiments, the detectable protein is a fluorescent protein. Examples of fluorescent proteins will be known by those of ordinary skill in the art and include, but are not limited to, mNeonGreen, GFP, EGFP, Superfold GFP, Azami Green, mWasabi, TagGFP, TurboGFP, acGFP, zsGreen, T-sapphire, EBFP, EBFP2, Azurite, TagBFP, ECFP, mECFP, Cerulean, mTurquoise, CyPet, AmCyan1, TagCFP, mTFP1, EYFP, mCitrine, TagYFP, phiYFP, zsYellow1, mBanana, Kusabira Orange, mOrange, dTomato, DsRed, mTangerine, mRuby, mApple, mStrawberry, AsRed2, mRFP1, mCherry, HcRed1, iRFP720, and AQ143.

In some embodiments, the detectable protein is a luciferase enzyme.

In some embodiments, the detectable protein is a β-galactosidase enzyme.

In some embodiments, the detectable protein is a molecule that is capable of being secreted by a cell.

In some embodiments, the detectable protein is a cell surface protein. In some embodiments, the cell surface protein serving as the detectable protein may be used to purify or separate cells from a heterogenous population.

In some embodiments, the detectable protein is an enzyme that is capable of producing a pigment that can be detected by eye. In some embodiments, the enzyme serving as the detectable protein produces a molecule comprising a unique photochemical signature. In some embodiments, the enzyme serving as a detectable protein may produce a small molecule that serves as a substrate in a chemical reaction that produces a pigment or a molecule comprising a unique photochemical signature.

In some embodiments, the detectable protein comprises an amino acid sequence having at least 80%, at least 85%, at least 90%, at least 92%, at least 95%, at least 98%, or at least 99% identity to any one of SEQ ID NOs: 14-15. In some embodiments, the detectable protein comprises or consists of the amino acid sequence of SEQ ID NOs: 14-15.

E. Reporter

In some embodiments, an RNA-responsive sensor described herein comprises a sequence encoding a reporter. As used herein, the term “reporter” refers to a detectable protein that can be used to identify the presence of a corresponding RNA-responsive sensor, even in the absence of a mutation the sensor sequence's premature stop codon. In some embodiments, an RNA-responsive sensor that comprises both a reporter and an output comprising a detectable protein yields two different signals in the cell upon translation of the output. In some embodiments, the reporter can be used to detect the presence of an RNA-responsive sensor in a cell. In some embodiments, a reporter can be used as a transfection marker.

In some embodiments, the reporter is located upstream of the sensor sequence and thus is not impacted by the stop codon in the sensor sequence. In some embodiments, the reporter is separated from the sensor sequence by a self-cleaving peptide sequence.

In some embodiments, the reporter is a fluorescent protein. Examples of fluorescent proteins will be known by those of ordinary skill in the art and include, but are not limited to, mNeonGreen, GFP, EGFP, Superfold GFP, Azami Green, mWasabi, TagGFP, TurboGFP, acGFP, zsGreen, T-sapphire, EBFP, EBFP2, Azurite, TagBFP, ECFP, mECFP, Cerulean, mTurquoise, CyPet, AmCyan1, TagCFP, mTFP1, EYFP, mCitrine, TagYFP, phiYFP, zsYellow1, mBanana, Kusabira Orange, mOrange, dTomato, DsRed, mTangerine, mRuby, mApple, mStrawberry, AsRed2, mRFP1, mCherry, HcRed1, iRFP720, smURFP, and AQ143.

In some embodiments, the reporter is an enzyme. In some embodiments, the reporter is a luciferase enzyme. In some embodiments, the reporter is a β-galactosidase enzyme. In some embodiments, the reporter is an enzyme that is capable of producing a pigment that can be detected by eye. In some embodiments, the enzyme serving as the reporter produces a molecule comprising a unique photochemical signature. In some embodiments, the enzyme serving as a reporter may produce a small molecule that serves as a substrate in a chemical reaction that produces a pigment or a molecule comprising a unique photochemical signature.

In some embodiments, the reporter is a cell surface protein. In some embodiments, the cell surface protein serving as the reporter may be used to purify or separate cells from a heterogenous population.

In some embodiments, the reporter comprises an amino acid sequence having at least 80%, at least 85%, at least 90%, at least 92%, at least 95%, at least 98%, or at least 99% identity to any one of SEQ ID NOs: 14-15. In some embodiments, the reporter comprises or consists of the amino acid sequence of SEQ ID NOs: 14-15.

F. Self-Cleaving Peptide

In some embodiments, an RNA-responsive sensor described herein comprises a sequence encoding a self-cleaving peptide. As used herein, “a self-cleaving peptide” refers to an amino acid sequence that promotes physical and chemical separation of the proteins encoded by the RNA-responsive sensor (such as an output sequence, a base editor that acts on dsRNA, and/or a reporter) such that the proteins are not linked via a continuous polypeptide sequence. As such, in some embodiments, the self-cleaving peptide sequences determine that individual proteins encoded by the engineered nucleic acid comprise their own free N- and C-terminal moieties that are not involved in the formation of a peptide bond between the proteins. In some embodiments, self-cleaving of a self-cleaving peptides occurs via ribosomal skipping of glycyl-prolyl peptide bond formation to yield two separate proteins.

In some embodiments, an RNA-responsive sensor described herein comprises one or more sequences encoding one or more self-cleaving peptides. In some embodiments, when an RNA-responsive sensor comprises more than one self-cleaving peptide sequences, the plurality of self-cleaving peptide sequences may be the same sequence located in different regions of the RNA-responsive sensor. In some embodiments, when an RNA-responsive sensor comprises more than one self-cleaving peptide sequences, the plurality of self-cleaving peptide sequences may be different (e.g., two, three, four, or more distinct self-cleaving peptide sequences).

Exemplary self-cleaving peptides are known to those having ordinary skill in the art. In some embodiments, a self-cleaving peptide is a 2A peptide. 2A peptides typically comprise 18-22 amino acids which can induce ribosomal skipping during translation of protein. The 2A peptide family comprises four members including P2A (derived from porcine teschovirus-1 2A), E2A (derived from equine rhinitis A virus), F2A (derived from foot-and-mouth disease virus), and T2A (derived from thosea asigna virus 2A). In some embodiments, members of the 2A family comprise a core sequence motif of DXEXNPGP (SEQ ID NO: 68). In some embodiments, the RNA-responsive sensors described herein comprise a self-cleaving peptide sequence comprising an amino acid sequence having at least 80%, at least 85%, at least 90%, at least 92%, at least 95%, at least 98%, or at least 99% identity with the amino acid sequence of any one of SEQ ID NOs: 10-12. In some embodiments, the RNA-responsive sensors described herein comprise a self-cleaving peptide sequence comprising or consisting of an amino acid of any one of SEQ ID NOs: 10-12.

In some embodiments, the self-cleaving peptide may comprise a linker on its N- or C-terminal end. In some embodiments, the linker added to the N- or C-terminal end of the self-cleaving peptide is an unstructured, glycine-rich peptide linker. In some embodiments, the unstructured, glycine-rich peptide linker comprises multiple repeats of a glycine-rich motif.

In some embodiments, the glycine-rich motif is of the sequence GGSG. In some embodiments, the unstructured, glycine-rich linker may comprise 1, 2, 3, 4, 5, 6, 7, 8, 9, or more repeats of a GGSG motif. In some embodiments, the self-cleaving peptide is a 2A peptide which may comprise a GSG linker on the N-terminal end.

In some embodiments, the self-cleaving peptide may comprise an intein sequence.

In some embodiments, the self-cleaving peptide may comprise a cleavage site for an endogenous protease (e.g., a wildtype protease or a disease associated protease, such as a protease expressed by cancer cells).

G. Exemplary RNA-Responsive Sensors

In some embodiments, an RNA-responsive sensor comprises a nucleic acid sequence having at least 80%, at least 85%, at least 90%, at least 92%, at least 95%, at least 98%, or at least 99% identity a nucleic acid sequence of any one of SEQ ID NOs: 21-47. In some embodiments, an RNA-responsive sensor comprises or consists of the nucleic acid sequence of any one of SEQ ID NOs: 21-47.

H. Percent Identity

As used herein, the term “percent identity” refers to a relationship between two nucleic acid sequences or two amino acid sequences, as determined by sequence comparison (alignment). In some embodiments, identity is determined across the entire length of a sequence. In some embodiments, identity is determined over a region of a sequence.

Identity of sequences can be readily calculated by those having ordinary skill in the art.

In some embodiments, the percent identity of two sequences is determined using the algorithm of Karlin and Altschul 1990 Proc. Natl. Acad. Sci. U.S.A. 87:2264-68, modified as in Karlin and Altschul 1993 Proc. Natl. Acad. Sci. U.S.A. 90:5873-77. This algorithm is incorporated into the NBLAST® and XBLAST® programs (version 2.0) of Altschul et al. 1990 J. Mol. Biol. 215:403-10. BLAST® protein searches can be performed, for example, with the XBLAST program, score=50, wordlength=3 to obtain amino acid sequences homologous to the protein molecules of the invention. Where gaps exist between two sequences, Gapped BLAST® can be utilized, for example, as described in Altschul et al. 1997 Nucleic Acids Res. 25(17):3389-3402. When utilizing BLAST® and Gapped BLAST® programs, the default parameters of the respective programs (e.g., XBLAST® and NBLAST®) can be used, or the parameters can be adjusted appropriately as would be understood by one of ordinary skill in the art.

### II. Nucleic Acids Encoding an RNA-Responsive Sensor

In some aspects, the disclosure relates to nucleic acids that encode an RNA-responsive sensor as described herein. In some embodiments, nucleic acid sequence encoding an RNA-responsive sensor is comprised within an expression cassette, such that the nucleic acid sequence encoding the RNA-responsive sensor is operably linked to a promoter and/or other regulatory sequences.

In some embodiments, “regulatory sequences” include, in a non-limiting manner, transcriptional regulatory sequences (e.g., promoter, enhancer, silencer, transcription factor binding sequence, 5′ UTR, or 3′ UTR), post-transcriptional regulatory sequences (e.g., acceptor/donor splicing sites and splicing regulatory sequences), and/or translation regulatory sequences (e.g., translation initiation signals, translation termination signals, mRNA degradation or decay signals, polyadenylation signals).

In some embodiments, regulatory sequences include, without limitation, promoter sequences, ribosome binding sites, ribozymes, enhancer sequences, response elements, protein recognition sites, inducible elements, protein binding sequences, 5′ and 3′ untranslated regions (UTRs), transcriptional start sites, transcription terminator sequences, polyadenylation sequences, introns, and premature stop codons.

The promoter driving expression of an RNA-responsive sensor can be, but is not limited to, a constitutive promoter, an inducible promoter, a tissue-specific promoter, or a synthetic promoter.

In some embodiments, the sequence encoding an RNA-responsive sensor is operably linked to a constative promoter. Constitutive promoters maintain constant expression of RNAs (e.g., RNAs corresponding to an RNA-responsive sensor) regardless of the conditions or physiological state of a host cell. In some embodiments, a constitutive promoter can be, but is not limited to, a Herpes Simplex virus (HSV) promoter, a thymidine kinase (TK) promoter, a Rous Sarcoma Virus (RSV) promoter, a Simian Virus 40 (SV40) promoter, a Mouse Mammary Tumor Virus (MMTV) promoter, an Adenovirus E1A promoter, a cytomegalovirus (CMV) promoter (see, e.g., Boshart et al., Cell, 41:521-530 (1985)), the phosphoglycerol kinase (PGK) promoter, the CAG promoter, and the human elongation factor-1 alpha (EF1α) promoter [Invitrogen], the dihydrofolate reductase promoter, a mammalian housekeeping gene promoter, or a β-actin promoter.

In some embodiments, the sequence encoding an RNA-responsive sensor is operably linked to an inducible promoter. Inducible promoters allow regulation of gene expression and can be regulated by exogenously supplied compounds, environmental factors such as temperature, or the presence of a specific physiological state. Inducible promoters and inducible systems are available from a variety of commercial sources, including, without limitation, Invitrogen, and Clontech. Many other systems have been described and can be readily selected by one of skill in the art. An inducible promoter can be, but is not limited to, an IPTG-inducible promoter, a cytochrome P450 gene promoter, a heat shock protein gene promoter, a metallothionein gene promoter, a hormone-inducible gene promoter, an estrogen gene promoter, or a tetVP16 promoter, the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)), the rapamycin-inducible system (Magari et al., J. Clin. Invest., 100:2865-2872 (1997)). Still other types of inducible promoters which may be useful in this context are those which are regulated by a specific physiological state.

In some embodiments, the sequence encoding an RNA-responsive sensor is operably linked to a tissue-specific promoter. In some cases, the tissue-specific regulatory sequences bind tissue-specific transcription factors that induce transcription in a tissue specific manner. Such tissue-specific regulatory sequences (e.g., promoters, enhancers, etc.) are well known in the art. Exemplary tissue-specific regulatory sequences include, but are not limited to the following tissue specific promoters: retinoschisin proximal promoter, interphotoreceptor retinoid-binding protein enhancer (RS/IRBPa), rhodopsin kinase (RK), liver-specific thyroxin binding globulin (TBG) promoter, an trypsin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (α-MHC) promoter, or a cardiac Troponin T (cTnT) promoter. Other exemplary promoters include Beta-actin promoter, hepatitis B virus core promoter, Sandig et al., Gene Ther., 3:1002-9 (1996); alpha-fetoprotein (AFP) promoter, Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998); immunoglobulin heavy chain promoter; T cell receptor α-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)), among others which will be apparent to the skilled artisan.

In some embodiments, the sequence encoding an RNA-responsive sensor is operably linked to a native promoter of a gene endogenous to a cell describe herein. In some embodiments, the native promoter may be preferred when it is desired that expression of the RNA-responsive sensor should mimic the native expression of a gene of interest. In some embodiments, the native promoter may be used when expression of the RNA-responsive sensor must be regulated temporally, developmentally, in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the promoter driving expression of an RNA-responsive sensor is an RNA pol II promoter. In some embodiments, the promoter is an RNA pol III promoter, such as U6 or H1. In some embodiments, the promoter is an RNA pol II promoter. In some embodiments, the promoter is a CMV enhancer (CMVe). In some embodiments, the promoter is a chicken β-actin (CBA) promoter. In some embodiments, the promoter is a CMVe and a CBA promoter. In some embodiments, the promoter is a CAG promoter. Other examples of promoters which may be operably linked to a sequence encoding an RNA-responsive sensor described herein include a BDNF promoter, an NGF promoter, an EGF promoter, a growth factor promoter, an axon-specific promoter, a dendrite-specific promoter, a brain-specific promoter, a hippocampal-specific promoter, a kidney-specific promoter, an elafin promoter, a cytokine promoter, an interferon promoter, an α1 antitrypsin promoter, a brain cell-specific promoter, a neural cell-specific promoter, a central nervous system cell-specific promoter, a peripheral nervous system cell-specific promoter, an interleukin promoter, a serpin promoter, a hybrid CMV promoter, a hybrid β-actin promoter, an EF1 promoter, a U1a promoter, a U1b promoter, a Tet-inducible promoter, a VP16 LexA promoter, or a mammalian or avian β-actin promoter.

In some embodiments, an expression cassette comprises a polyadenylation sequence following the sequence encoding the RNA-responsive sensor and before any other 3′ regulatory sequence (e.g., a 3′ AAV ITR). In some embodiments, a poly(A) signal sequence is inserted following the sequence encoding the RNA-responsive sensor and before any other 3′ regulatory sequence (e.g., a 3′ AAV ITR), which signals for the polyadenylation of transcribed mRNA molecules. Examples of poly(A) signal sequences include, but are not limited to, bovine growth hormone (bGH) poly(A) signal sequence, SV-40 poly(A) signal sequence, and synthetic poly(A) signal sequences, which are known to cause polyadenylation of eukaryotic transgenes and efficient termination of translation (Azzoni A R et al., J Gene Med. 2007; 9(5):392-402).

In some embodiments, a sequence that enhances expression of the RNA-responsive sensor may further be inserted following the sequence encoding the RNA-responsive sensor sequence and before the 3′ AAV ITR and poly(A) signal sequences. An exemplary sequence includes, but is not limited to, a woodchuck hepatitis virus (WHV) post-transcriptional regulatory element (WPRE) (Higashimoto T et al., Gene Ther. 2007; 14(17):1298-304).

As used herein, a nucleic acid sequence (e.g., coding sequence) and regulatory sequences are said to be “operably linked” when they are covalently linked in such a way as to place the expression or transcription of the nucleic acid sequence under the influence or control of the regulatory sequences. For example, if it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly, two or more coding regions are operably linked when they are linked in such a way that their transcription from a common promoter result in the expression of two or more proteins having been translated in frame.

A. Vectors

In some embodiments, a nucleic acid that encodes an RNA-responsive sensor as described herein (or a corresponding cassette) is a vector. As such, aspects of the present disclosure relate to vectors for the expression of RNA-responsive sensors. The term “vector” or “expression vector” or “construct” means any molecular vehicle, such as a plasmid, phage, transposon, recombinant viral genome, cosmid, chromosome, artificial chromosome, virus, viral particle, viral vector (e.g., lentiviral vector or AAV vector), virion, etc. which can transfer gene sequences (e.g., an engineered nucleic acid encoding an ADAR-mediated RNA-responsive sensor) into a cell or between cells. In some embodiments, a viral vector is a lentivirus vector comprising a transgene comprising the nucleic acid sequence of an RNA-responsive sensor flanked by a first and a second lentivirus long-inverted terminal repeat (LTR). In some embodiments, a viral is a recombinant adeno-associated virus (rAAV) vector comprising a transgene comprising the nucleic acid sequence of an RNA-responsive sensor flanked by a first and a second AAV inverted terminal repeat (ITR).

In some embodiments, vectors are single-stranded. In some embodiments, vectors are double-stranded. In some embodiments, vectors are circular (e.g., circular plasmids, nanoplasmids, and minicircle plasmids). In some embodiments, vectors are linear. In some embodiments, vectors are self-complementary.

In some embodiments, the vector may be maintained in high levels in a cell using a selection method such as involving an antibiotic resistance gene. In some embodiments, the vector may comprise a partitioning sequence which ensures stable inheritance of the vector. In some embodiments, the vector is a high copy number vector. In some embodiments, the vector becomes integrated into a chromosome of a cell.

B. Recombinant Viral Genomes and Recombinant Viruses

In some embodiments, a vector is a recombinant viral vector. As such, aspects of the present disclosure relate to recombinant viral genomes for the expression of RNA-responsive sensors. In some embodiments, a recombinant viral vector is a recombinant adeno-associated virus (rAAV) vector. In some embodiments, a recombinant viral vector is a recombinant lentivirus vector. As used herein, “transgene” refers to a DNA sequence (e.g., comprising a nucleic acid sequence of an RNA-responsive sensor) which encodes an RNA to be expressed in a cell.

“Recombinant AAV (rAAV) vectors” typically comprise, at a minimum, a transgene (e.g., comprising the nucleic acid sequence of an RNA-responsive sensor) including its regulatory sequences, flanked by 5′ and 3′ AAV inverted terminal repeats (ITRs). In some embodiments, the 5′ and 3′ ITRs may be alternatively referred to as “first” and “second” ITRs, respectively. The rAAVs of the present disclosure may comprise a transgene comprising an RNA-responsive sensor coding sequence region in addition to expression control sequences (e.g., a promoter, an enhancer, a poly(A) signal, etc.), as described elsewhere in this disclosure.

In some embodiments, a rAAV vector comprises, from 5′ to 3′, a first adeno-associated virus (AAV) inverted terminal repeat (ITR) sequence, a promoter operably linked to the sequence encoding the RNA-responsive sensor, a polyadenylation signal, and a second AAV inverted terminal repeat (ITR) sequence. In some embodiments, the rAAV vector is circular. In some embodiments, the rAAV vector is linear. In some embodiments, the rAAV vector genome is single-stranded. In some embodiments, the rAAV vector genome is double-stranded. In some embodiments, the rAAV genome vector comprising is a self-complementary rAAV vector.

Inverted terminal repeat (ITR) sequences are about 145 bp in length. While the entire sequences encoding the ITRs are commonly used in engineering rAAVs, some degree of minor modification of these sequences is permissible. The ability to modify these ITR sequences is within the capabilities of one of ordinary skill in the pertinent the art. (See, e.g., texts such as Sambrook et al., Molecular Cloning. A Laboratory Manual, 2d ed., Cold Spring Harbor Laboratory, New York (1989); and K. Fisher et al., J Virol., 70:520 532 (1996)).

rAAV particles disclosed herein, may be of any AAV serotype. Examples of AAV serotypes include AAV1, AAV2, AAV3, AAV4, AAV5, AAV6, AAV7, AAV8, AAV9, and AAV10. Non-limiting examples of rAAV pseudotypes include AAV2/1, AAV2/5, AAV2/8, AAV2/9, AAV3/1, AAV3/5, AAV3/8, and AAV3/9, wherein the slash denotes an rAAV genome comprising the transgene flanked by ITRs of one serotype packaged in the capsid comprising a AAV capsid protein from a different serotype (e.g., an rAAV genome comprising AAV2 ITRs packaged in a capsid with an AAV capsid protein from AAV5 would be AAV2/5). As used herein, the serotype of an rAAV refers to the serotype of the capsid proteins of the recombinant virus. Non-limiting examples of derivatives and/or other types include, but are not limited to, AAVrh.10, AAVrh.74, AAV2-AAV3 hybrid, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAE1/2, AAV clone 32/83, AAVShHIO, AAV2 (Y->F), AAV8 (Y733F), AAV2.15, AAV2.4, AAVM41, and AAVr3.45.

Such AAV serotypes and derivatives/pseudotypes, and methods of producing such derivatives/pseudotypes are known in the art (see, e.g., Mol. Ther. 2012 April; 20(4):699-708. Doi: 10.1038/mt.2011.287. Epub 2012 Jan. 24. The AAV vector toolkit: poised at the clinical crossroads. Asokan Al, Schaffer D V, Samulski R J.). Methods for producing and using pseudotyped rAAV vectors are known in the art (see, e.g., Duan et al, J. Virol., 75:7662-7671, 2001; Halbert et al, J. Virol., 74:1524-1532, 2000; Zolotukhin et al, Methods, 28:158-167, 2002; and Auricchio et al., Hum. Molec. Genet., 10:3075-3081, 2001). In some embodiments, the rAAV comprising an ADAR-mediated RNA-responsive sensor is pseudotyped.

In some embodiments, the components needed by a host cell to package an rAAV genome in a capsid may be provided to the host cell in trans. In some embodiments, any one or more of the required components (e.g., the AAV rep sequences and AAV cap sequences, and/or helper functions) may be provided by a stable host cell which has been engineered to contain one or more of the required components. In some embodiments, such a stable host cell will contain the required component(s) under the control of either an inducible promoter or a constitutive promoter. Examples of suitable inducible and constitutive promoters are provided herein. The methods used to construct any rAAV particle have also been previously described (see, e.g., Sambrook et al., Molecular Cloning: A Laboratory Manual, Cold Spring Harbor Press, Cold Spring Harbor, NY (Fourth Edition, 2014)). Similarly, methods of generating rAAV particles are well known and the selection of a suitable method is not a limitation on this disclosure (see, e.g., K. Fisher et al., J. Virol., 70:520-532 (1993) and U.S. Pat. No. 5,478,745).

In some embodiments, rAAV particles may be produced using the triple transfection method (described in detail in U.S. Pat. No. 6,001,650). In some embodiments, rAAV particles are produced by transfecting a host cell with an AAV vector (comprising a transgene flanked by ITR elements) to be packaged into rAAV particles, a packaging nucleic acid (e.g., a nucleic acid encoding the AAV rep and AA cap sequences), and a AAV helper nucleic acid.

An AAV helper nucleic acid comprises the AAV helper function sequences, such as E1, E2A, E4, and/or VA. Preferably, rAAV particles are produced without any detectable wild-type AAV virus particles (e.g., AAV virus particles containing functional rep and cap genes). Helper nucleic acids, and methods of making the same, have been previously described and are commercially available (see, e.g., pDM, pDG, pDP1rs, pDP2rs, pDP3rs, pDP4rs, pDP5rs, pDP6rs, pDG(R484E/R585E), and pDP8.ape plasmids from PlasmidFactory, Bielefeld, Germany; other products and services available from Vector Biolabs, Philadelphia, PA; Cellbiolabs, San Diego, CA; Agilent Technologies, Santa Clara, Ca; and Addgene, Cambridge, MA; pxx6; Grimm et al. (1998), Novel Tools for Production and Purification of Recombinant Adeno-associated Virus Vectors, Human Gene Therapy, Vol. 9, 2745-2760; Kern, A. et al. (2003), Identification of a Heparin-Binding Motif on Adeno-Associated Virus Type 2 Capsids, Journal of Virology, Vol. 77, 11072-11081; Grimm et al. (2003), Helper Virus-Free, Optically Controllable, and Two-Plasmid-Based Production of Adeno-associated Virus Vectors of Serotypes 1 to 6, Molecular Therapy, Vol. 7, 839-850; Kronenberg et al. (2005), A Conformational Change in the Adeno-Associated Virus Type 2 Capsid Leads to the Exposure of Hidden VP1 N Termini, Journal of Virology, Vol. 79, 5296-5303; and Moullier, P. and Snyder, R. O. (2008), International efforts for recombinant adeno-associated viral vector reference standards, Molecular Therapy, Vol. 16, 1185-1188). In some embodiments, the helper nucleic acid may be provided in a helper virus, such as adenovirus, herpes virus (other than herpes simplex virus type-1), and vaccinia virus. The packing nucleic acid comprises nucleotide sequences AAV rep and AAV cap gene sequences. In some embodiments, functions of the helper and packaging nucleic acids support AAV replication, activation of AAV gene transcription, stage specific AAV mRNA splicing, AAV DNA replication, synthesis of cap expression products, and/or AAV capsid assembly.

“Recombinant lentivirus vectors” at a minimum, a transgene (e.g., comprising the nucleic acid sequence of an RNA-responsive sensor) including its regulatory sequences, flanked by 5′ and 3′ lentivirus long inverted terminal repeats (LTRs). In some embodiments, the 5′ and 3′ LTRs may be alternatively referred to as “first” and “second” LTRs, respectively. In some embodiments, recombinant lentiviruses of the present disclosure may comprise a transgene comprising an RNA-responsive sensor coding sequence region in addition to expression control sequences (e.g., a promoter, an enhancer, a poly(A) signal, etc.), as described elsewhere in this disclosure.

The lentivirus is a retrovirus, meaning it has a single stranded RNA genome with a reverse transcriptase enzyme, which functions to perform transcription of the viral genetic material upon entering the cell. Lentiviruses also have a viral envelope with protruding glycoproteins that aid in attachment to the outer membrane of a host cell.

Within the lentivirus genome are RNA sequences that code for specific proteins that facilitate the incorporation of the viral sequences into genome of a host cell. The ends of the genome are flanked with LTRs. LTRs are necessary for integration of the dsDNA into the host chromosome. LTRs also serve as part of the promoter for transcription of the viral genes.

Non-limiting examples of proteins encoded by the lentiviral genome include gag, pol, env, tat, etc. The “gag” gene codes for the structural components of the viral nucleocapsid proteins: the matrix (MA/p17), the capsid (CA/p24), and the nucleocapsid (NC/p7) proteins. The “pol” domain codes for the reverse transcriptase and integrase enzymes. Lastly, the “env” domain of the viral genome encodes the glycoproteins of the envelope found on the surface of the virus.

In some embodiments, to produce a recombinant lentiviral particle, a suitable host cell is contacted with a plurality of nucleic acids (e.g., a nucleic acid comprising the transgene flanked by LTRs, one or more packaging nucleic acids, and/or an envelope nucleic acid). In some embodiments, a packaging nucleic acid comprises gag and/or pol sequences. In some embodiments, a packaging nucleic acid further comprises tat and/or rev sequences. In some embodiments, two packaging nucleic acids are used wherein one comprises gag and/or pol sequences and another comprises the rev sequence. In some embodiments, an envelope nucleic acid comprising env sequences is used. In some embodiments, the one or more packaging nucleic acids and/or the envelope nucleic acid comprise lentiviral sequences under the control of a promoter.

Additional examples of lentiviruses, lentivirus vectors, and methods of use thereof are described in U.S. Pat. Nos. 8,420,104 B2, 5,994,136, 6,207,455 B1, WO2006/089001 A2, Merten et al., Methods Clin. Dev., 3:16017 (2016), Sakuma et al., Biochem. J., 443(3):603-618 (2012), Tiscornia et al., Nature Prot., 1:241-245 (2006).

### III. Genetic Circuits

In some aspects, the disclosure relates to genetic circuits comprising: (i) an RNA-responsive sensor as described in Part I or a nucleic acid encoding the same the same, as described in Part II; and (ii) a trigger nucleic acid (a ssRNA molecule). In some embodiments, in the circuits described herein, a region of the trigger nucleic acid (i.e., the trigger sequence) is capable of hybridizing with the sensor sequence of the RNA-responsive sensor, however a nucleotide of the premature stop codon of the sensor sequence is non-complementary to the trigger nucleic acid. In some embodiments, when translated, the base editor that acts on dsRNA, and that is encoded by the RNA-responsive sensor, is capable of editing the non-complementary nucleotide of the premature stop codon (e.g., UAG) such that it is converted into an amino acid-coding codon (e.g., UIG).

In some embodiments, the genetic circuits described herein are in an OFF state, when translation of the output of the RNA-responsive sensor cannot occur, even in the presence of translation machinery (because of the existence of the premature stop codon in the sensor sequence of the ADAR-mediated RNA-responsive sensor). In some embodiments, the genetic circuits described herein are in an ON state, when translation of the output can occur in the presence of translation machinery (because of mutation of the premature stop codon in the sensor sequence to an amino acid-coding codon, as expounded upon below).

As used herein, “translation machinery” refers to ribosomes and associated factors necessary for protein biosynthesis, such as tRNA(s), amino acid(s), and elongation factors. In some embodiments, the engineered cells described herein (see Part IV) endogenously comprise translation machinery necessary for protein biosynthesis.

A. Trigger Nucleic Acid

In some embodiments, the genetic circuits described herein comprise a trigger nucleic acid. In some embodiments, a trigger nucleic acid, like an RNA-responsive sensor, is an RNA molecule. In some embodiments, trigger nucleic acids can be any RNA molecule.

In some embodiments, a trigger nucleic acid is a small RNA such as an RNA that is less than 200 ribonucleotides in length. In some embodiments, a trigger nucleic acid is 10-20, 20-30, 30-40, 40-50, 50-60, 60-70, 70-80, 80-90, 90-100, 100-110, 110-120, 120-130, 130-140, 140-150, 150-160, 160-170, 170-180, 180-190, or 190-199 nucleotides in length.

In some embodiments, a trigger nucleic acid is a long RNA such as an RNA that is 200 or more nucleotides in length. In some embodiments, a trigger nucleic acid is 200-250, 250-300, 300-350, 350-400, 400-450, 450-500, 500-550, 550-600, 600-650, 650-700, 700-750, 750-800, 800-850, 850-900, 900-950, 950-1,000, 1,000-1,500, 1,500-2,000, 2,000-2,500, 2,500-3,000, 3,000-3,500, 3,500-4,000, 4,000-4,500, 4,500-5,000, or more nucleotides in length.

In some embodiments, a trigger nucleic acid is a naturally occurring RNA, synthetic RNA, endogenous RNA, or exogenous RNA. In some embodiments, a trigger nucleic acid is a messenger RNA (mRNA), a transfer RNA (tRNA), a ribosomal RNA (rRNA), a guide RNA (gRNA), a CRISPR RNA (crRNA) trans-activating CRISPR RNA (tracrRNA), or a non-coding RNA (ncRNA), such as a long non-coding RNA (lncRNA), a small-interfering RNA (siRNA), a small-hairpin RNA (shRNA), a small nucleolar RNA (snoRNA), a micro RNA (miRNA), or a Piwi-interacting RNA (piRNA).

In some embodiments, a trigger nucleic acid is a non-coding RNA (e.g., a non-coding RNA of a cell, e.g., a cell described in Part IV).

In some embodiments, a trigger nucleic acid is an mRNA (e.g., an mRNA of a cell, e.g., a cell described in Part IV). In some embodiments, a trigger nucleic acid is an mRNA of a secreted protein.

In some embodiments, the trigger nucleic acids described herein comprise a trigger sequence. As used herein, a “trigger sequence” refers to a region of a target nucleic acid that is capable of hybridizing to a sensor sequence of an RNA-responsive sensor. In some embodiments, a trigger nucleic acid is an mRNA, and the trigger sequence is within a coding sequence of the mRNA. In some embodiments, a trigger nucleic acid is an mRNA, and the trigger sequence is a splice junction between two exons of the mRNA. In some embodiments, a trigger nucleic acid is an mRNA, and the trigger sequence comprises, or comprises a sequence within, an exon respective to an alternative splice variant of the mRNA of the gene encoding the trigger nucleic acid. In some embodiments, a trigger nucleic acid is an mRNA, and the trigger sequence is within a non-coding region of the mRNA. For example, in some embodiments, a trigger nucleic acid is an mRNA, and the trigger sequence is within the 3′ UTR of the mRNA. In some embodiments, a trigger nucleic acid is an mRNA, and the trigger sequence is within the 5′ UTR of the mRNA. In some embodiments, a trigger nucleic acid is an mRNA, and the trigger sequence is within an intron of the mRNA.

Trigger sequences vary in length. In some embodiments, a trigger sequence is at least 10, at least 15, at least 20, at least 25, at least 30, at least 35, at least 40, at least 50, at least 60, at least 70, at least 80, at least 90, at least 100, at least 120, at least 140, at least 160, at least 180, or at least 200 nucleotides in length. In some embodiments, a trigger sequence is no more than 200, no more than 180, no more than 160, no more than 140, no more than 120, no more than 100, no more than 90, no more than 80, no more than 70, no more than 60, no more than 50, no more than 40, no more than 25, or no more than 20 nucleotides in length. In some embodiments, a trigger sequence is 10-15, 15-20, 20-25, 25-30, 30-35, 35-40, 40-45, 45-50, 50-55, 55-60, 60-65, 65-70, 70-75, 75-80, 80-85, 85-90, 90-95, 95-100, 100-110, 110-120, 120-130, 130-140, 140-150, 150-160, 160-170, 170-180, 180-190, 190-200, 200-225, 225-250, 250-275, or 275-300 nucleotides in length. In some embodiments, a trigger sequence is 50 or 75 nucleotides in length.

In some embodiments, the trigger nucleic acids described herein comprise a nucleotide sequence having at least 80%, at least 85%, at least 90%, at least 92%, at least 95%, at least 98%, or at least 99% identity to any one of SEQ ID NOs: 16-20. In some embodiments, the trigger nucleic acids described herein comprise the nucleotide sequence of any one of SEQ ID NOs: 16-20.

B. Hybridization of a Trigger Sequence with a Sensor Sequence

In some embodiments, the sensor sequence of an RNA-responsive sensor of the circuits described herein is designed such that it is capable of hybridizing (e.g., under the physiological conditions of a cell) with a trigger sequence of a trigger nucleic acid (e.g., a trigger nucleic acid of a cell). Hybridization of the senor sequence of an RNA-responsive sensor with a trigger sequence of a trigger nucleic acid generates a “region of double-stranded RNA (dsRNA).” The sensor sequence recognizes the trigger nucleic acid by hybridizing to the trigger nucleic acid thereby forming a region of dsRNA, wherein one strand is contributed by the sensor sequence and one strand is contributed by the trigger nucleic acid. As such, in some embodiments, a sensor sequence will comprise a nucleic acid sequence having a degree of complementarity relative to a trigger sequence of a trigger nucleic acid that is sufficient to promote binding (e.g., under the physiological conditions of a cell). In some embodiments, a “region of dsRNA” comprises at least 15, at least 16, at least 17, at least 18, at least 19, at least 20, at least 21, at least 22, at least 23, at least 24, at least 25, at least 26, at least 27, at least 28, at least 29, at least 30, at least 35, at least 40, or at least 50 complimentary RNA nucleotides (i.e., base pairing nucleotides between the sensor sequence and the trigger sequence). In some embodiments, a region of dsRNA comprises no more than 50, no more than 40, no more than 35, no more than 30, or no more than 25 complementary RNA nucleotides. In some embodiments, a region of dsRNA comprises 15-50, 15-45, 15-40, 15-35, 15-30, 15-25, 20-50, 20-45, 20-40, 20-35, 20-30, 30-50, 30-45, or 30-40 complimentary RNA nucleotides. In some embodiments, a region of dsRNA comprises 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more complimentary RNA nucleotides.

In some embodiments, initial hybridization of a sensor sequence with a corresponding trigger sequence is imperfect, at least because at least one nucleotide (preferably one nucleotide) of the premature stop codon of the sensor sequence is designed to form a mismatch with the corresponding trigger sequence. Hybridization between a sensor sequence and a trigger sequence may be imperfect for additional reasons. For example, in some embodiments, a sensor sequence comprises one or more functional nucleic acid motif(s) (as described above), which do not hybridize with a trigger sequence (see e.g., FIG. 3A).

C. Activation of a Genetic Circuit

As described above, in some embodiments, the genetic circuits described herein are in an OFF state, when translation of the output of the RNA-responsive sensor cannot occur, even in the presence of appropriate translation machinery (because of the existence of the premature stop codon in the sensor sequence of the RNA-responsive sensor). In some embodiments, the genetic circuits described herein are in an ON state (or activated), when translation of the output can occur in the presence of appropriate translation machinery.

In some embodiments, activation of a genetic circuit described herein occurs when the premature stop codon of a sensor sequence is mutated to an amino acid-coding codon, thereby enabling translation (in the presence of translation machinery) of the sequence encoding a base editor that acts on dsRNA and the sequence encoding the output of the RNA-responsive sensor. In some embodiments, mutation of the premature stop codon occurs when a base editor that acts on dsRNA (e.g., an ADAR expressed by a cell) binds to the region of dsRNA formed between a sensor sequence and its corresponding trigger sequence and mutates the premature stop codon such that it becomes an amino acid-coding codon. In so embodiments, improper base pairing between the sensor sequence and the trigger nucleic acid sequence recruits a base editor that acts on dsRNA which edits the sensor sequence thereby abolishing the premature stop codon.

For example, in some embodiments, the sensor sequence comprises a premature stop codon that forms an improper A:C base pair with the trigger sequence, wherein the A is contributed by the sensor sequence and the C is contributed by the trigger sequence. In some embodiments, the sensor sequence comprises a premature stop codon comprising the sequence 5′-UAG-3′ which binds to a 5′-CCA-3′ sequence in the trigger sequence thereby forming an improper A:C base pair. In some embodiments, the sensor sequence comprises a premature stop codon comprising the sequence 5′-UGA-3′ which binds to a 5′-CCA-3′ sequence in the trigger nucleic acid thereby forming an improper A:C base pair. In some embodiments, following deamination (e.g., via a base editor that acts on double stranded RNA having adenosine deaminase activity), the adenosine of the improper A:C base pair will be converted to a I thereby encoding a 5′-UIG-3′ in the sensor sequence, which is read as UGG (a codon for tryptophan).

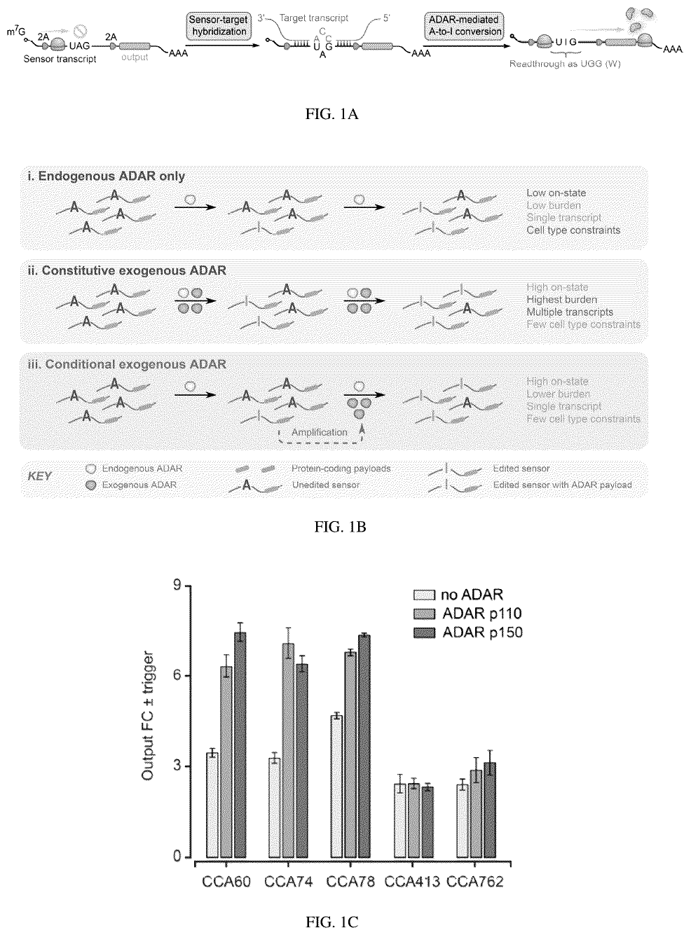

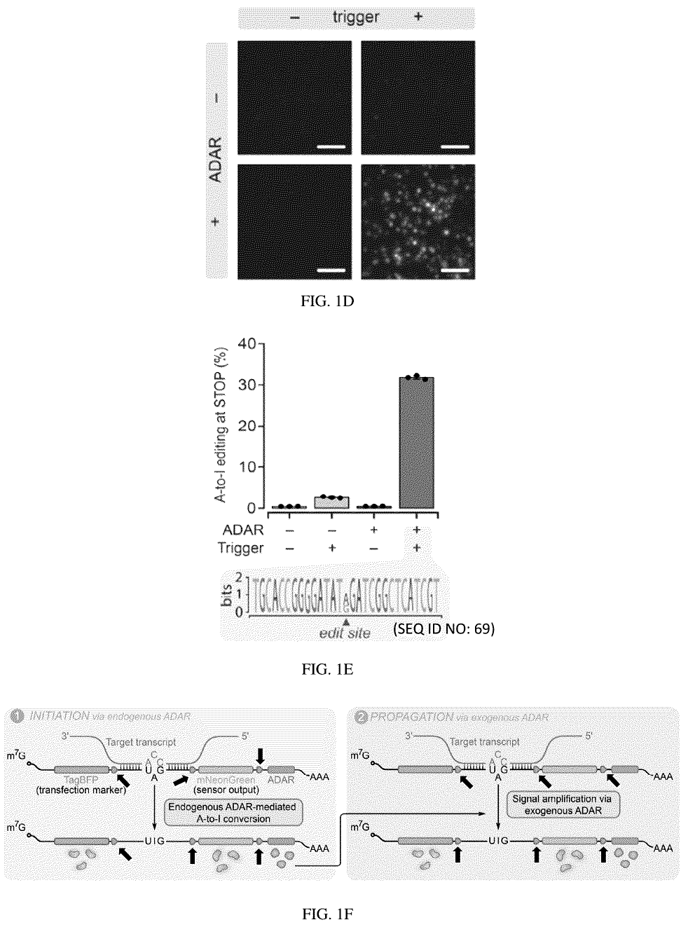

In some embodiments, a genetic circuit is self-amplifying such that a change in structure, regulation, and/or activity of one RNA-responsive sensor results in a change in the structure, regulation, and/or activity of another RNA-responsive sensor thereby forming a positive-feedback loop. In some embodiments, the genetic circuits described herein are autocatalytic, as mutation of a premature stop codon of a sensor sequence results in translation of the sequence encoding a base editor that acts on dsRNA of the RNA-responsive sensor (in the presence of translation machinery), which is designed such that it can act on (i.e., bind and mutate) additional RNA-responsive sensors that may be present (see e.g., FIG. 1B). In some embodiments, the positive feedback loop occurs amongst the same species of RNA-responsive sensors (i.e., identical RNA-responsive sensors, e.g., having the same output). In some embodiments, the said positive feedback loop occurs amongst different species of RNA-responsive sensors (i.e., different RNA-responsive sensors, e.g., having different outputs).

### IV. Cells Comprising an RNA-Responsive Sensor

In some aspects, the disclosure relates to cells comprising an RNA-responsive sensor described herein or a nucleic acid encoding the same. In some embodiments, a cell may endogenously express a trigger nucleic acid comprising a trigger sequence corresponding to a sensor sequence of the RNA-responsive sensor. In some embodiments, the cell may also express (endogenously or exogenously) a base editor that acts on dsRNA and that is capable of mutating the premature stop codon of the RNA-responsive sensor (e.g., an ADAR protein). In some embodiments, an RNA-responsive sensor is provided to a cell comprising low levels of endogenous base editor that acts on double-stranded RNAs.

In some embodiments, a cell comprises a plurality of RNA-responsive sensors (or nucleic acids encoding the same). In some embodiments, the plurality of RNA responsive-sensors (or nucleic acids encoding the same) comprises at least 2, at least 3, at least 4, at least 5, at least 6, at least 7, at least 8, at least 9, or at least 10 distinct RNA-responsive sensors (i.e., RNA responsive-sensors having unique sequences).

An RNA-responsive sensor (or a nucleic acid encoding the same) may be introduced into a cell using methods commonly used by those having ordinary skill in the art. In some embodiments, an RNA-responsive sensor (or nucleic acid encoding the same) is introduced into a cell using recombinant DNA techniques. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197 which are incorporated by reference herein in their entirety. In some embodiments, a nucleic acid encoding an RNA-responsive sensor is introduced into the genome of a cell.

The cells described herein may be derived from any cell. In some embodiments, the cell may be a prokaryotic cell. In some embodiments, the cell may be a eukaryotic cell. In some embodiments, the eukaryotic cell may be a plant cell or fungal cell (e.g., a yeast). In some embodiments, the cell may be a mammalian cell, such as a human cell, a chicken cell, or an insect cell. Examples of suitable mammalian cells are, but are not limited to, HEK-293T cells, COS7 cells, Hela cells and HEK-293 cells. Examples of suitable insect cells include, but are not limited to, High5 cells and Sf9 cells. In some embodiment, the cells are insect cells as they are devoid of undesirable human proteins, and their culture does not require animal serum. Other examples of host cells, such as mammalian cells include, primate cells (e.g., Vero cells), and rat cells (e.g., GH3 cells, 0C23 cells) or mouse cells (e.g., MC3T3 cells). Host cells also include mammalian stem cells (e.g., human stem cells or human neural stem cells) such as, for example, pluripotent stem cells (e.g., human pluripotent stem cells including human induced pluripotent stem cells (hiPSCs)). A stem cell refers to a cell with the ability to divide for indefinite periods in culture and to give rise to specialized cells. A pluripotent stem cell refers to a type of stem cell that is capable of differentiating into all tissues of an organism, but not alone capable of sustaining full organismal development. A human induced pluripotent stem cell refers to a somatic (e.g., mature or adult) cell that has been reprogrammed to an embryonic stem cell-like state by being forced to express genes and factors important for maintaining the defining properties of embryonic stem cells (see, e.g., Takahashi and Yamanaka, Cell 126 (4): 663-76, 2006). Human induced pluripotent stem cells express stem cell markers and are capable of generating cells characteristic of all three germ layers (ectoderm, endoderm, mesoderm). In some embodiments, a cell may be a primary cell. In some embodiments, a cell may be located in a subject (e.g., a human patient). In some embodiments, cells may be derived from cell lines including 293T, 3T3, 4T1, 721, 9L, A-549, A172, A20, A253, A2780, A2780ADR, A2780cis, A431, ALC, B16, B35, BCP-1, BEAS-2B, bEnd.3, BHK-21, BR 293, BxPC3, C2C12, C3H-10T1/2, C6, C6/36, Cal-27, CGR8, CHO, CML T1, CMT, COR-L23, COR-L23/5010, COR-L23/CPR, COR-L23/R23, COS-7, COV-434, CT26, D17, DH82, DU145, DuCaP, E14Tg2a, EL4, EM2, EM3, EMT6/AR1, EMT6/AR10.0, FM3, H1299, H69, HB54, HB55, HCA2, Hepa1c1c7, High Five cells, HL-60, HMEC, HT-29, HUVEC, J558L cells, Jurkat, JY cells, K562 cells, KCL22, KG1, Ku812, KYO1, LNCap, Ma-Mel 1, 2, 3 . . . 48, MC-38, MCF-10A, MCF-7, MDA-MB-231, MDA-MB-435, MDA-MB-468, MDCK II, MG63, MONO-MAC 6, MOR/0.2R, MRCS, MTD-1A, MyEnd, NALM-1, NCI-H69/CPR, NCI-H69/LX10, NCI-H69/LX20, NCI-H69/LX4, NIH-3T3, NW-145, OPCN/OPCT Peer, PNT-1A/PNT 2, PTK2, Raji, RBL cells, RenCa, RIN-5F, RMA/RMAS, S2, Saos-2 cells, Sf21, Sf9, SiHa, SKBR3, SKOV-3, T-47D, T2, T84, THP1, U373, U87, U937, VCaP, WM39, WT-49, X63, YAC-1, and YAR cells. C8161, CCRF-CEM, MOLT, mIMCD-3, NHDF, HeLa-S3, Huh1, Huh4, Huh7, HASMC, HEKn, HEKa, MiaPaCell, Pancl, PC-3, TF1, CTLL-2, C1R, Rat6, CV1, RPTE, A10, T24, J82, A375, ARH-77, Calul, SW480, SW620, SK-UT, CaCo2, P388D1, SEM-K2, WEHI-231, HB56, TIB55, J45.01, LRMB, Bcl-1, BC-3, IC21, DLD2, Raw264.7, NRK, NRK-52E, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A 172, A20, A253, A431, A-549, ALC, B16, B35, BCP-1 cells, BEAS-2B, bEnd.3, BHK-21, BR 293. BxPC3. C3H-10T1/2, C6/36, Cal-27, CHO, CHO-7, CHO-IR, CHO-K1, CHO-K2, CHO-T, CHO Dhfr −/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK 11, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. In some embodiments, a cell may be derived from a cell line selected from a list consisting of: RBL cells, RenCa, RIN-5F, RMA/RMAS, S2, Saos-2 cells, Sf21, Sf9, SiHa, SKBR3, SKOV-3, T-47D, T2, T84, THP1, U373, U87, U937, VCaP, WM39, WT-49, X63, YAC-1, and YAR cells. C8161, and CCRF-CEM.

### V. Compositions

In some aspects, the disclosure relates to compositions comprising a (or a plurality of) RNA-responsive sensor(s) and/or a nucleic acid sequence(s) encoding the same.

In some embodiments, a composition further comprises a liposome, a lipid, a lipid complex, a lipid nanoparticle, a microsphere, a microparticle, a nanosphere, and/or a nanoparticle, or may be otherwise formulated for administration to the cells, biological samples, tissues, organs, or body of a subject in need thereof.

In some embodiments, a composition further comprises a pharmaceutical excipient. Pharmaceutically acceptable excipients (excipients) are substances other than a therapeutic agent that are intentionally included in a drug delivery system. In some embodiments, excipients do not exert or are not intended to exert a therapeutic effect. In some embodiments, excipients may act to a) aid in processing of the drug delivery system during manufacture, b) protect, support or enhance stability, bioavailability or patient acceptability of the API, c) assist in product identification, and/or d) enhance any other attribute of the overall safety, effectiveness, or delivery of the therapeutic agent during storage or use. In some embodiments, a pharmaceutically acceptable excipient may be an inert substance. In some embodiments, a pharmaceutically acceptable excipient may not be an inert substance. Excipients include, but are not limited to, absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

In some embodiments, a composition further comprises additional components commonly found in pharmaceutical compositions. Such additional components can include, but are not limited to: anti-pruritics, astringents, local anesthetics, or anti-inflammatory agents (e.g., antihistamine, diphenhydramine).

In some embodiments, compositions comprising an RNA-responsive sensor may be suitable for treatment regimens and thereby administered to a subject via a variety of methods described herein. Such compositions may be formulated for use in a variety of therapies, such as, for example, in the amelioration, prevention, and/or treatment of conditions. Accordingly, in some embodiments, the compositions comprising an RNA-responsive sensor described herein may be administered to a subject such as human or non-human subjects, a host cell in situ in a subject, a host cell ex vivo, a host cell derived from a subject, or a biological sample (e.g., one derived from a subject).

In some embodiments, for administration of an injectable aqueous solution, the composition comprising an RNA-responsive sensor may be suitably buffered, if necessary, and the liquid diluent first rendered isotonic with sufficient saline, polyalcohols, or glucose. For example, in some embodiments, one dosage of a therapeutic agent may be dissolved in an isotonic NaCl solution and optionally added to a larger volume of hypodermoclysis fluid prior to being injected at the proposed site of infusion. In some embodiments, the composition comprising an RNA-responsive sensor is provided in a solvent or dispersion medium containing, for example, water, ethanol, polyol (for example, glycerol, propylene glycol, and liquid polyethylene glycol), and suitable mixtures thereof. In some embodiments, a composition may also contain adjuvants such as preservatives, wetting agents, emulsifying agents, and dispersing agents. In some embodiments, proper fluidity may be maintained, for example, by the use of a coating, such as lecithin, by the maintenance of the required particle size in the case of dispersion and by the use of surfactants.

### VI. Kits

In some aspects, the disclosure relates to kits comprising a (or a plurality of) RNA-responsive sensor(s) and/or a nucleic acid sequence(s) encoding the same.

In some embodiments, the kits described herein may include one or more containers housing components for performing the methods described herein, and optionally instructions for use. In some embodiments, the components may be prepared sterilely, packaged in a syringe, and shipped refrigerated. Alternatively, in some embodiments, they may be housed in a vial or other container for storage. In some embodiments, a second container may have other components prepared sterilely. Alternatively, in some embodiments, the kits may include the active agents premixed and shipped in a vial, tube, or other container. In some embodiments, the kits may also include other components, depending on the specific application, for example, containers, cell media, salts, buffers, reagents, syringes, needles, a fabric, such as gauze, for applying or removing a disinfecting agent, disposable gloves, a support for the agents prior to administration, etc.

In some embodiments, any of the kits described herein may further comprise components needed for inducing uptake of an RNA-responsive sensor (or nucleic acid encoding the same) into a cell. In some embodiments, each component of the kits, where applicable, may be provided in liquid form (e.g., in solution) or in solid form, (e.g., a dry powder). In some embodiments, some of the components may be reconstitutable or otherwise processible (e.g., to an active form), for example, by the addition of a suitable solvent or other species (for example, water), which may or may not be provided with the kit.

In some embodiments, a kit further comprises a set of instructions for carrying out the methods described herein. As used herein, “instructions” can define a component of instruction and/or promotion, and typically involve written instructions on or associated with packaging of this disclosure. In some embodiments, instructions also can include any oral or electronic instructions provided in any manner such that a user will clearly recognize that the instructions are to be associated with the kit, for example, audiovisual (e.g., videotape, DVD, etc.), Internet, and/or web-based communications, etc. In some embodiments, the written instructions may be in a form prescribed by a governmental agency regulating the manufacture, use, or sale of pharmaceuticals or biological products, which can also reflect approval by the agency of manufacture, use or sale for animal administration. As used herein, “promoted” includes all methods of doing business including methods of education, hospital and other clinical instruction, scientific inquiry, drug discovery or development, academic research, pharmaceutical industry activity including pharmaceutical sales, and any advertising or other promotional activity including written, oral, and electronic communication of any form, associated with this disclosure. Additionally, in some embodiments, the kits may include other components depending on the specific application, as described herein.

In some embodiments, the kits may have a variety of forms, such as a blister pouch, a shrink-wrapped pouch, a vacuum sealable pouch, a sealable thermoformed tray, or a similar pouch or tray form, with the accessories loosely packed within the pouch, one or more tubes, containers, a box, or a bag. In some embodiments, the kits may be sterilized after the accessories are added, thereby allowing the individual accessories in the container to be otherwise unwrapped. In some embodiments, the kits, or any of its components, can be sterilized using any appropriate sterilization techniques, such as radiation sterilization, heat sterilization, or other sterilization methods known in the art.

### VII. Methods

In some aspects, the disclosure relates to methods that utilize a RNA-responsive sensor or a nucleic acid encoding the same. In some embodiments, the RNA-responsive sensor sequences described herein may be designed in order to impart various functional capabilities. In some embodiments, an RNA-responsive sensor is designed for diagnostic purposes. In some embodiments, an RNA-responsive sensor sequence is designed to determine if a person has developed a disease, disorder, or condition or if a person previously diagnosed with a disease, disorder, or condition is no longer exhibiting disease, disorder, or condition-associated symptoms. In some embodiments, an RNA-responsive sensor can be designed to distinguish if a cell expresses a specific isoform of a protein. In some embodiments, a plurality of RNA-responsive sensors comprising distinct sensor sequences may be employed in order to determine the relative amounts of specific isoforms of a protein in a cell. In some embodiments, an RNA-responsive sensor sequences is designed to distinguish healthy cells from disease cells in a mosaic tissue.

In some embodiments, an RNA-responsive sensor may be designed to target expression a therapeutic protein to cells that express a corresponding trigger nucleic acid and an endogenous base editor that acts on double-stranded RNA. In some embodiments, a therapeutic protein, when expressed, is capable of modifying the sequence of a gene found within in the cell's genome (e.g., by introducing a therapeutic or corrective mutation in the gene).

As used herein, a “therapeutic protein” leads to a physiological change that is associated with or expected to at least partially, if not fully, remedy at least one symptom associated with a disease, disorder, or condition. A therapeutic protein refers to any proteinaceous molecule that is translated from an RNA expressed from a transgene which is therapeutic upon translation in a target cell. In some embodiments, the therapeutic protein may be therapeutic for any disease, disease, or condition described herein upon administration to a subject in need thereof. Non-limiting examples of therapeutic proteins include enzymes (such as proteases, signaling proteins, transcriptional regulators, CRISPR/Cas endonucleases such as Cas9, base editors, prime editors, etc.), enzymatic domains, enzyme substrates, hormones, receptors (e.g., chimeric antigen receptors), components of gene editing ribonucleoprotein complexes (e.g., CRISPR/Cas endonucleases such as Cas9, base editors, prime editors, etc.), peptibodies, growth factors, clotting factors, cytokines, chemokines, activating or inhibitory peptides acting on cell surface receptors or ion channels, cell-permeable peptides targeting intracellular processes, thrombolytics, bone morphogenetic proteins, Fc-fusion proteins, anticoagulants, and antibodies or antigen-binding fragments thereof. In some embodiments, a therapeutic protein is selected for the purposes of gene replacement therapy.

In some embodiments, these methods may utilize pluralities of RNA-responsive sensors (or nucleic acid encoding the same), including RNA-responsive sensors that have distinct sequences. In some embodiments, a plurality comprises 1, 2, 3, 4, 5, 6, 7, 8, 9, 10 or more different RNA-responsive sensors (or nucleic acids encoding the same).

A. Methods of Treating

In some aspects, the disclosure relates to methods of treating or preventing a disease, disorder, or condition in a subject. In some embodiments, the method comprises administering to the subject an RNA-responsive sensor or a nucleic acid sequence encoding the same (as described herein). In some embodiments, the subject is administered a vector comprising a nucleic acid sequence encoding the RNA-responsive sensor. In some embodiments, the subject is administered a recombinant viral genome comprising a nucleic acid sequence encoding the RNA-responsive sensor, optionally wherein the recombinant viral genome is a recombinant adeno-associated virus (AAV) genome. In some embodiments, the subject is administered an rAAV particle comprising a transgene comprising a nucleic acid sequence encoding the RNA-responsive sensor.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. In some embodiments, the non-human animal may be a male or female at any stage of development. In some embodiments, the non-human animal may be a transgenic animal or genetically engineered animal. When a subject comprises a mutation the subject may be referred to as a mutant subject. The term “patient” refers to a human subject in need of treatment of a disease.

In some embodiments, administration of an RNA-responsive sensor achieves one, two, three, four, or more of the following effects, including, for example: (i) reduction or amelioration the severity of disease, disorder, or condition or symptom associated therewith; (ii) reduction in the duration of a symptom associated with a disease, disorder, or condition; (iii) protection against the progression of a disease or disorder or symptom associated therewith; (iv) regression of a disease, disorder, or condition or symptom associated therewith; (v) protection against the development or onset of a symptom associated with a disease, disorder, or condition; (vi) protection against the recurrence of a symptom associated with a disease; (vii) reduction in the hospitalization of a subject; (viii) reduction in the hospitalization length; (ix) an increase in the survival of a subject with a disease; (x) a reduction in the number of symptoms associated with a disease, disorder, or condition; (xi) an enhancement, improvement, supplementation, complementation, or augmentation of the prophylactic or therapeutic effect(s) of another therapy. In some embodiments, the disease, disorder, or condition comprises a genetic disease, cancer, inflammatory disease or a inflammatory condition, autoimmune disease, liver disease, spleen disease, lung disease, hematological disease, neurological disease, painful condition, psychiatric disorder, metabolic disorder, immune disorder, infection by a pathogen, a kidney disease, cardiovascular disease, pancreatic disease, intestinal disease, retinal disease, neuromuscular disease, musculoskeletal disease, lysosomal storage disease, or other disease, or any combination thereof.

In some embodiments, it will be desirable to deliver an RNA-responsive sensor (or nucleic acid encoding the same) subcutaneously, intraocularly, intravitreally, parenterally, subcutaneously, intravenously, intracerebro-ventricularly, intramuscularly, intracranially, intrathecally, orally, intraperitoneally, or by oral or nasal inhalation, or by direct injection to one or more cells, tissues, or organs. In some embodiments, an RNA-responsive sensor (or nucleic acid encoding the same) is injected directly into the cerebrospinal fluid of the subject. In some embodiments, direct injection is performed concurrently with a surgical procedure or interventional procedure. In general, the most appropriate route of administration will depend upon a variety of factors including the nature of the agent (e.g., its stability in the environment of the gastrointestinal tract), and/or the condition of the subject (e.g., whether the subject is able to tolerate oral administration, injection, etc.). In some embodiments, compositions are administered to a subject through only one administration route. In some embodiments, multiple administration routes may be exploited (e.g., serially, or simultaneously) for administration of the composition to a subject.

B. Methods of Detecting an RNA Molecule in a Sample

In some aspects, the disclosure relates to methods of detecting an RNA molecule in a sample. In these embodiments, the output of an RNA-responsive sensor will preferably comprise a detectable protein as described above in Part ID.

In some embodiments, the method comprises: a) contacting a sample with an RNA-responsive sensor or a nucleic acid encoding the same (as described herein), wherein the sample comprises: (i) a base editor that acts on double stranded RNA or a polynucleotide encoding the same; and (2) mRNA translation machinery; and b) detecting output that is produced.

In some embodiments, the sample is a biological sample. The term “biological sample” refers to any sample including tissue samples (such as tissue sections and needle biopsies of a tissue, such as those comprising living neural cells); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include brain tissues, blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample. In some embodiments, a biological sample may be derived from a human subject, cultured in vitro, contacted with an engineered nucleic acid, and then introduced into either the same human subject or a different human subject.

In some embodiments, step a) comprises introducing the engineered nucleic acid, the vector, or the recombinant viral genome into a cell, wherein the cell comprises: (i) a base editor or a polynucleotide encoding the same; and (2) mRNA translation machinery. In some embodiments, step a) comprises introducing an rAAV particle comprising a transgene comprising a nucleic acid sequence encoding an RNA-responsive sensor.

Various methods for detecting the presence of an output (e.g., a detectable protein) will be available to those of skill in the art. Such methods may include, but are not limited to, confocal microscopy, fluorescence microscopy, flow cytometry, fluorescent activated cell sorting (FACS), mass spectrometry, gas chromatography, gas chromatography-mass spectrometry, thin paper liquid chromatography, western blot, enzyme-link immunosorbent assay (ELISA), and absorbance measurement.

C. Methods of Expressing a Product of Interest

In some aspects, the disclosure relates to methods of producing a product of interest in a cell. In some embodiments, the method comprises introducing into the cell an RNA-responsive sensor or nucleic acid encoding the same, wherein the output of the RNA-responsive sensor comprises the protein of interest, and wherein the cell expresses: (i) a trigger nucleic acid comprising a trigger sequence, wherein the trigger sequence is capable of hybridizing with the sensor sequence of the RNA-responsive sensor, and wherein a nucleotide of the premature stop codon of the sensor sequence is non-complementary to the trigger sequence; and (ii) an endogenous base editor that is capable of editing the nucleotide of the premature stop codon of the sensor sequence that is non-complementary to the trigger sequence, optionally wherein the base editor comprises an ADAR.

D. Methods of Producing an RNA-Responsive Sensor

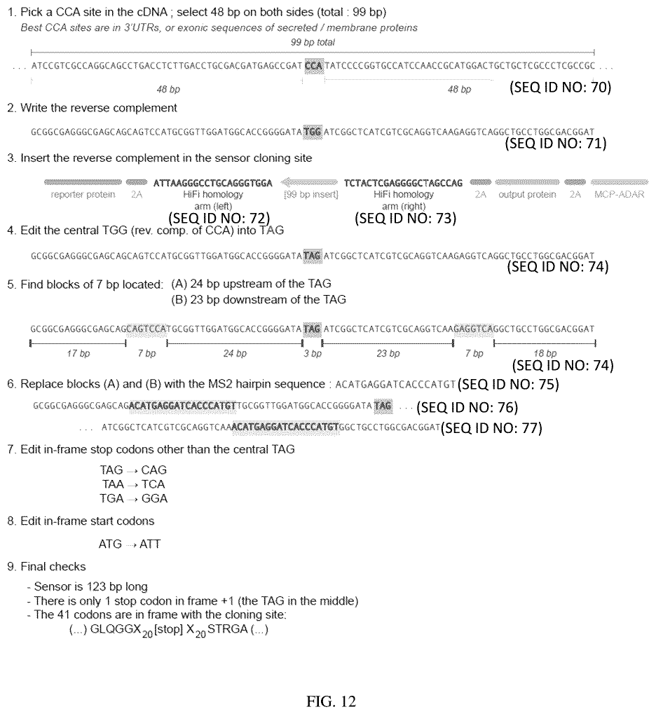

In some aspects, the disclosure relates to methods of producing an RNA-responsive sensor comprising: (a) designing and RNA responsive sensor; and (b) synthesizing the RNA responsive sensor. In some embodiments, step (a) comprises one or more of the steps depicted in FIG. 12. In some embodiments, step (a) comprises each of the steps depicted in FIG. 12. Methods of synthesizing an RNA-responsive sensor are known to those having ordinary skill in the art. Exemplary methods of synthesizing an RNA-responsive sensor are provided in the Examples section.

### EXAMPLES

### Example 1

This example describes a non-limiting example of an autocatalytic circuit comprising an RNA-responsive sensor that can be activated by ADAR (i.e., an ADAR-mediated RNA-responsive sensor) when the ADAR is endogenously expressed in a cell. The autocatalytic circuit includes a single RNA transcript encoding an output (e.g., a product of interest) and ADAR itself to edit additional sensor molecules present in the cell (see, FIG. 1B, panel iii). The circuit forms a positive feedback loop, amplifying the signal from endogenously-expressed ADAR.

### Methods:

Cloning: For the ADAR-mediated RNA-responsive sensor expression plasmids, custom entry vectors were made by isothermal assembly of dsDNA fragments using the NEBuilder HiFi DNA assembly mix (NEB #E2621). Fragments were generated by PCR using high-fidelity Q5 polymerase (NEB #M0494), with in-house plasmids and custom-synthesized gBlocks (Integrated DNA Technologies) as templates. The entry vectors were designed such that the fluorescent protein expression cassettes harbor a multiple cloning site (MCS) without in-frame stop codons, insulated from the fluorescent proteins by sequences coding for 2A peptides. To assemble the final ADAR-mediated RNA-responsive sensor plasmids, sensor sequences were ordered as long oligonucleotides (Sigma Aldrich) with 5′ and 3′ adapter sequences overlapping with the vectors around the HindIII site of the MCS. Oligonucleotides were made double-stranded by PCR and inserted the resulting dsDNA products into HindIII-linearized entry vectors using HiFi assembly mix. To build ADAR-expressing plasmids, plasmids pmGFP-ADAR1-p150 and pmGFP-ADAR1-p110 (Addgene #117927 and #117928, respectively) were used as a starting point. The GFP sequences were excised from the plasmids by amplifying the backbones with Q5 polymerase before circularizing the PCR products with KLD mix (NEB #M0554). The MCP-ADAR sequence was amplified from plasmid MS2-adRNA-MCP-ADAR2DD(E488Q)-NES, kindly provided by Prashant Mali (Addgene #124705). After each cloning and transformation step, the regions of interest in individual clones were verified by Sanger sequencing (QuintaraBio, Azenta). All of the plasmids were propagated in Escherichia coli Turbo (NEB #C2984) or Stable (NEB #C3040) strains, with 100 μg/mL carbenicillin (Teknova #C2110) for selection.

Human cell culture: Cryopreserved HEK293FT cells were obtained from Invitrogen (#R70007) and maintained in Dulbecco's modification of Eagle's medium (DMEM, Gibco #10569010) supplemented with 10% v/v fetal bovine serum (FBS, Gibco #16000044) and 1×MEM non-essential amino acids (Gibco #11140050). Both wild-type and MALAT1 knock-out A459 cells as previously described (31) were propagated in Ham's F-12K (Kaighn's) Medium (Gibco #21127030) supplemented with 10% v/v FBS.

All the cells were grown in a humidified atmosphere at 37° C. with 5% CO₂, and split using trypsin-EDTA (Gibco #25300054) every 2-3 days to ensure they did not surpass 80% confluence. Cells were used at low passage numbers (<15) for all experiments.

C2C12 cell culture and differentiation: C2C12 cells were obtained from ATCC (CRL-1772) and maintained in culture in DMEM supplemented with 10% v/v FBS. Care was taken to ensure that they did not exceed 50% confluence. For differentiation to the muscle lineage, cells were allowed to become fully confluent (which we defined as day 0) one day after transfections, at which point the growth medium was switched to DMEM supplemented with 2% v/v horse serum (Cytiva #SH3007402) and 1× insulin-transferrin-selenium supplement (Sigma Aldrich #I3146).

The growth medium was replaced every 48 hr until the end of the differentiation experiment. For differentiation to the bone lineage the cells were grown in DMEM+10% v/v FBS supplemented with 1000 ng/mL recombinant BMP-2 (R&D Systems #355BECO25) for 5 days prior to transfection with plasmids.

Transfections: Lipofectamine 3000 (Invitrogen #L3000015) was used for transient transfections. Cells were transfected at 70-90% confluence. In each well of a 96-well plate, a total of 150 ng plasmid DNA was transfected, which included 50 ng of each plasmid (sensor, ADAR, and/or trigger). When leaving out one or several plasmids, the mass of transfected plasmids were standardized by adding a filler plasmid (carrying an Fluc2 gene with or without a promoter). For each well, 0.5 μL of P3000 reagent was diluted in a final volume of 5 μL of OptiMEM (Gibco #51985091), as well as 0.5 μL of Lipofectamine in 5 μL of OptiMEM. For larger culture vessels, the transfections were scaled up according to the area of the plates. The cells were analyzed 48 hr after transfection.

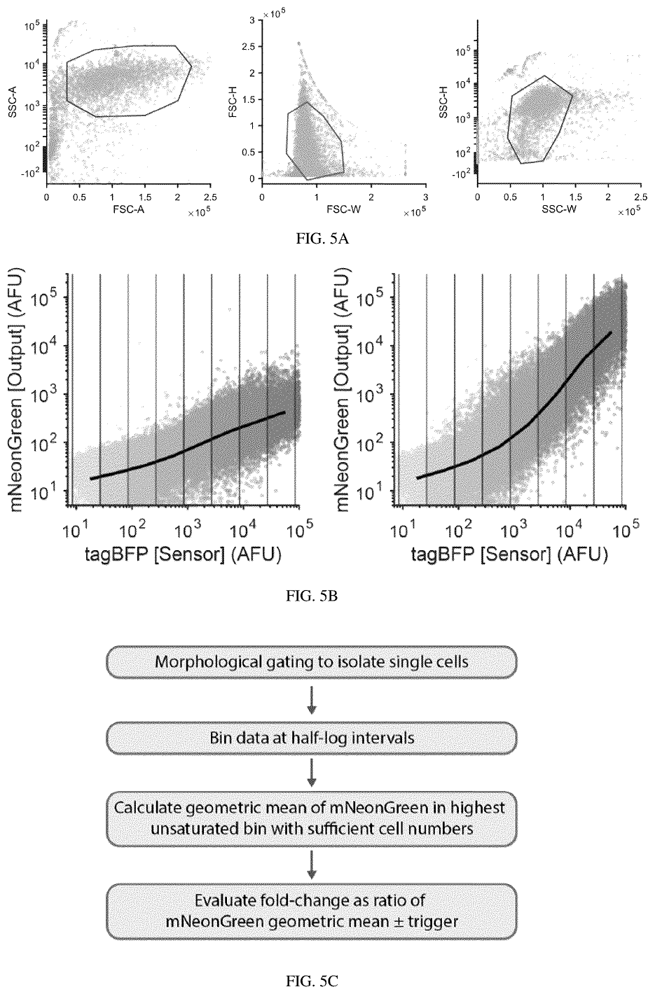

Fluorescence analyses: Fluorescent protein expression was analyzed by flow cytometry. To do so, cells were harvested 48 hr after transfection using trypsin-EDTA. The cells were washed three times with flow cytometry buffer, made of phosphate buffered saline without calcium or magnesium (Corning #21031CV) supplemented with 1% FBS and 5 mM EDTA. Cells were kept on ice until analysis with the HTS module of a BD LSR-II flow cytometer (Koch Institute flow cytometry core). The data was analyzed using Matlab scripts (based on github.com/jonesr18/MATLAB_Flow_Analysis). As a general strategy, cell populations were binned according to their transfection levels, at half-log intervals in the TagBFP-Pacific Blue channel (See, FIGS. 5A-5C).

Microscopy: For the imaging of HEK293FT cells, cells were transfected in a tissue-culture treated polystyrene 24-well plate. After 48 hr, the growth medium of the transfected cells was replaced with Hank's balanced salts solution without phenol red (Sigma Aldrich #H6648) and proceeded with the imaging at room temperature. Images were collected on a Nikon Ti-E inverted microscope equipped with a Nikon CFI S Plan Fluor ELWD 20×0.45 NA objective. A Nikon Intensilight C-HGFIE mercury lamp was used for illumination with the following filters for mNeonGreen: a 470/40 excitation filter and a 425/50 emission filter (Chroma #49002). Images were acquired with a Hamamatsu ORCA-Flash 4.0 CMOS camera controlled with NIS Elements AR 4.13.05 software.

RNA editing analysis: HEK293FT cells were transfected in triplicates in 6-well plates with the appropriate plasmids. After 48 hr, the cells were harvested with trypsin-EDTA; the cells were then washed with flow cytometry buffer and used fluorescence-activated cell sorting (FACS) to isolate transfected cells (TagBFP-positive cells, detected in the Pacific Blue channel). The cells were sorted directly in the lysis buffer from the Qiagen RNeasy Mini kit (#74106) and stored the homogenized samples at −80° C. until total RNA extraction following the manufacturer's instructions. A third-party company (Quintara Biosciences) produced cDNAs by reverse transcription of the sensor regions using an EasyQuick RT MasterMix (Cwbio #CW2019M) and primers CCA60-2264F/CCA60-2463R described in Table 8, after which the samples were sequenced on an Illumina MiSeq platform using a MiSeq Reagent Nano Kit v2 (300-cycles).

Editing efficiency was estimated by aligning the reads of each sample using Geneious mapper at medium sensitivity with up to 5 iterations per alignment and used a custom Matlab script to detect A-to-G substitutions at each nucleotide position.

Off-target editing analysis: Samples were prepared for RNA sequencing using a Qiagen RNeasy Plus mini kit (#74136). mRNAs were enriched using NEBNext Poly(A) mRNA Magnetic Isolation Module, followed by workup with NEBNext UltraII Directional RNA Library Prep Kit for Illumina. Sequences were sequenced on an Illumina MiSeq platform using a MiSeq Reagent Micro Kit v2 (2×150-cycles). Reads were trimmed using Fastp (32), indexed then, and STAR33 was used to align the reads to the UCSC hg38 reference genome (34) and annotations from Gencode (35). The sorted BAM files were sorted for A-to-I edits using REDItools v1.3 (36). The parameters can be found online at github.com/joncchen/dart_vadar.

Quantification of gene expression: At each timepoint, cells were harvested from 16 wells of a 96-well plate or a single well of a 6-well plate, depending on the experiment, using 1 mL of Tri-reagent RT (Molecular Research Center #RT111), and the samples were vortexed vigorously for 5 min. Samples were stored in the Tri-reagent at −80° C. until extraction. After thawing the samples, 50 μL of 4-bromoanisole (Thermo Scientific #A1182422) were added to the homogenate, vortexed and stored the samples on ice for 5 min prior to a 21,000 rcf centrifugation at 4° C. for 15 min. From the upper, clear aqueous phase, 500 μL of harvested cells were thoroughly mixed 1:1 with isopropanol. The mixture was applied on a silica spin column (Epoch Life Science #1910), spun and washed three times, once with buffer RW1 (Qiagen #1053394) and twice with buffer RPE (Qiagen #1018013). The samples were eluted in nuclease-free water and checked the RNA quality and concentration using a Nanodrop spectrophotometer. About 100 ng of total RNA was used in each RT-qPCR reaction using the Luna Universal One-Step RT-qPCR Kit (NEB #E3005), set up according to manufacturer's instructions and analyzed on a CFX Opus 96 instrument (Bio-Rad) in the SYBR-green channel. C2C12 gene expression was normalized within each biological sample to the levels of the housekeeping gene Csnk2a2. Primer sequences are available in Table 8.

Luciferase assays: At each time point of interest, samples were harvested from the wells transfected with the combinations of plasmids of interest. In each well of a 96-well plate containing 100 μL of growth medium, another 100 μL of Nano-Glo lysis/reaction buffer (Promega #N1110/N3040) reconstituted following manufacturer's recommendations was added. Vigorous pipetting was used to ensure complete homogenization and incubated the samples for 5 min at room temperature; then 150 μL of each sample was transferred to a white-bottom 96-well plate and measured luminescence on a ClarioStar Plus instrument (BMG Labtech) set with an acquisition window of 480/70 nm.

Staining: C2C12 cells were stained with Hoechst 33342 (Thermo Fisher #62249) and carboxyfluorescein succinimidyl ester (CFSE, Invitrogen #65085084) during differentiation to the muscle lineage. 1 μL Hoechst staining solution (20 mM stock) and 1 μL of 1000×CFSE (10 mM stock in DMSO) were diluted in 1 mL PBS and added 100 μL of this working staining solution to one well in a 96-well plate. The samples were incubated at room temperature protected from light for 10 minutes. Afterwards, the wells were washed three times with PBS prior to imaging on an EVOS M5000 microscope equipped with DAPI and GFP light cubes (Invitrogen #AMEP4950, AMEP4951). To generate images overlaying the DAPI (Hoechst) and GFP (CFSE) channels, the “Merge channels” function in Fiji 2 was used. For the functional evaluation of alkaline phosphatase expression in C2C12s treated with BMP-2, the cells were fixed with a paraformaldehyde-based buffer (Biolegend #420801) for 10 minutes at room temperature protected from light. Then, the cells were washed with water and subsequently stained with nitro blue tetrazolium chloride and 5-bromo-4-chloro-3-indolyl-1-phosphate (BCIP/NBT, Sigma Aldrich #AB0300). The samples were washed again with water, prior to imaging with an iPhone 12 mini (dual 12 MP, f/1.6 aperture, and iOS 15.5 software) mounted on a light transmission microscope.

Sequences: The following sequences relate to sensors, trigger nucleic acids, or experimental reagents described herein.

TABLE 1
Nucleic Acid Sequences

| | | SEQ |
|---|---|---|
| Sequence Type | Sequence | ID NO: |
| MS2 hairpin 1 | ACAUGAGGAUCACCCAUGU | 1 |
| MS2 hairpin 2 | ACAUGAGGAUUACCCAUGU | 2 |

TABLE 2
Protein Sequences

| | | SEQ |
|---|---|---|
| Sequence | | ID |
| Type | Sequence | NO: |
| human | MAEIKEKICDYLFNVSDSSALNLAKNIGLTKARDINAVLIDMERQGDVYRQG | 3 |
| ADAR1, | TTPPIWHLTDKKRERMQIKRNTNSVPETAPAAIPETKRNAEFLTCNIPTSNAS | |
| isoform | NNMVTTEKVENGQEPVIKLENRQEARPEPARLKPPVHYNGPSKAGYVDFEN | |
| p110 | GQWATDDIPDDLNSIRAAPGEFRAIMEMPSFYSHGLPRCSPYKKLTECQLKN | |
| | PISGLLEYAQFASQTCEFNMIEQSGPPHEPRFKFQVVINGREFPPAEAGSKKV | |
| | AKQDAAMKAMTILLEEAKAKDSGKSEESSHYSTEKESEKTAESQTPTPSATS | |
| | FFSGKSPVTTLLECMHKLGNSCEFRLLSKEGPAHEPKFQYCVAVGAQTFPSV | |
| | SAPSKKVAKQMAAEEAMKALHGEATNSMASDNQPEGMISESLDNLESMMP | |
| | NKVRKIGELVRYLNTNPVGGLLEYARSHGFAAEFKLVDQSGPPHEPKFVYQ | |
| | AKVGGRWFPAVCAHSKKQGKQEAADAALRVLIGENEKAERMGFTEVTPVT | |
| | GASLRRTMLLLSRSPEAQPKTLPLTGSTFHDQIAMLSHRCFNTLTNSFQPSLL | |
| | GRKILAAIIMKKDSEDMGVVVSLGTGNRCVKGDSLSLKGETVNDCHAEIISR | |
| | RGFIRFLYSELMKYNSQTAKDSIFEPAKGGEKLQIKKTVSFHLYISTAPCGDG | |
| | ALFDKSCSDRAMESTESRHYPVFENPKQGKLRTKVENGEGTIPVESSDIVPT | |
| | WDGIRLGERLRTMSCSDKILRWNVLGLQGALLTHFLQPIYLKSVTLGYLFSQ | |
| | GHLTRAICCRVTRDGSAFEDGLRHPFIVNHPKVGRVSIYDSKRQSGKTKETS | |
| | VNWCLADGYDLEILDGTRGTVDGPRNELSRVSKKNIFLLFKKLCSFRYRRDL | |
| | LRLSYGEAKKAARDYETAKNYFKKGLKDMGYGNWISKPQEEKNFYLCPV | |
| human | MNPRQGYSLSGYYTHPFQGYEHRQLRYQQPGPGSSPSSFLLKQIEFLKGQLP | 4 |
| ADAR1, | EAPVIGKQTPSLPPSLPGLRPRFPVLLASSTRGRQVDIRGVPRGVHLGSQGLQ | |
| isoform | RGFQHPSPRGRSLPQRGVDCLSSHFQELSIYQDQEQRILKFLEELGEGKATTA | |
| p150 | HDLSGKLGTPKKEINRVLYSLAKKGKLQKEAGTPPLWKIAVSTQAWNQHSG | |
| | VVRPDGHSQGAPNSDPSLEPEDRNSTSVSEDLLEPFIAVSAQAWNQHSGVVR | |
| | PDSHSQGSPNSDPGLEPEDSNSTSALEDPLEFLDMAEIKEKICDYLFNVSDSSA | |
| | LNLAKNIGLTKARDINAVLIDMERQGDVYRQGTTPPIWHLTDKKRERMQIK | |
| | RNTNSVPETAPAAIPETKRNAEFLTCNIPTSNASNNMVTTEKVENGQEPVIKL | |
| | ENRQEARPEPARLKPPVHYNGPSKAGYVDFENGQWATDDIPDDLNSIRAAP | |
| | GEFRAIMEMPSFYSHGLPRCSPYKKLTECQLKNPISGLLEYAQFASQTCEFN | |
| | MIEQSGPPHEPRFKFQVVINGREFPPAEAGSKKVAKQDAAMKAMTILLEEAK | |
| | AKDSGKSEESSHYSTEKESEKTAESQTPTPSATSFFSGKSPVTTLLECMHKLG | |
| | NSCEFRLLSKEGPAHEPKFQYCVAVGAQTFPSVSAPSKKVAKQMAAEEAMK | |
| | ALHGEATNSMASDNQPEGMISESLDNLESMMPNKVRKIGELVRYLNTNPVG | |
| | GLLEYARSHGFAAEFKLVDQSGPPHEPKFVYQAKVGGRWFPAVCAHSKKQ | |
| | GKQEAADAALRVLIGENEKAERMGFTEVTPVTGASLRRTMLLLSRSPEAQP | |
| | KTLPLTGSTFHDQIAMLSHRCFNTLTNSFQPSLLGRKILAAIIMKKDSEDMGV | |
| | VVSLGTGNRCVKGDSLSLKGETVNDCHAEIISRRGFIRFLYSELMKYNSQTA | |
| | KDSIFEPAKGGEKLQIKKTVSFHLYISTAPCGDGALFDKSCSDRAMESTESRH | |
| | YPVFENPKQGKLRTKVENGEGTIPVESSDIVPTWDGIRLGERLRTMSCSDKIL | |
| | RWNVLGLQGALLTHFLQPIYLKSVTLGYLFSQGHLTRAICCRVTRDGSAFED | |
| | GLRHPFIVNHPKVGRVSIYDSKRQSGKTKETSVNWCLADGYDLEILDGTRGT | |
| | VDGPRNELSRVSKKNIFLLFKKLCSFRYRRDLLRLSYGEAKKAARDYETAKN | |
| | YFKKGLKDMGYGNWISKPQEEKNFYLCPV | |
| human | MDIEDEENMSSSSTDVKENRNLDNVSPKDGSTPGPGEGSQLSNGGGGGPGR | 5 |
| ADAR2 | KRPLEEGSNGHSKYRLKKRRKTPGPVLPKNALMQLNEIKPGLQYTLLSQTGP | |
| | VHAPLFVMSVEVNGQVFEGSGPTKKKAKLHAAEKALRSFVQFPNASEAHLA | |
| | MGRTLSVNTDFTSDQADFPDTLFNGFETPDKAEPPFYVGSNGDDSFSSSGDL | |
| | SLSASPVPASLAQPPLPVLPPFPPPSGKNPVMILNELRPGLKYDFLSESGESHA | |
| | KSFVMSVVVDGQFFEGSGRNKKLAKARAAQSALAAIFNLHLDQTPSRQPIPS | |
| | EGLQLHLPQVLADAVSRLVLGKFGDLTDNFSSPHARRKVLAGVVMTTGTD | |
| | VKDAKVISVSTGTKCINGEYMSDRGLALNDCHAEIISRRSLLRFLYTQLELYL | |
| | NNKDDQKRSIFQKSERGGFRLKENVQFHLYISTSPCGDARIFSPHEPILEGSRS | |
| | YTQAGVQWCNHGSLQPRPPGLLSDPSTSTFQGAGTTEPADRHPNRKARGQL | |
| | RTKIESGEGTIPVRSNASIQTWDGVLQGERLLTMSCSDKIARWNVVGIQGSLL | |
| | SIFVEPIYFSSIILGSLYHGDHLSRAMYQRISNIEDLPPLYTLNKPLLSGISNAEA | |
| | RQPGKAPNFSVNWTVGDSAIEVINATTGKDELGRASRLCKHALYCRWMRV | |
| | HGKVPSHLLRSKITKPNVYHESKLAAKEYQAAKARLFTAFIKAGLGAWVEK | |
| | PTEQDQFSLTP | |
| human | MASVLGSGRGSGGLSSQLKCKSKRRRRRRSKRKDKVSILSTFLAPFKHLSPGI | 6 |
| ADAR3 | TNTEDDDTLSTSSAEVKENRNVGNLAARPPPSGDRARGGAPGAKRKRPLEE | |
| | GNGGHLCKLQLVWKKLSWSVAPKNALVQLHELRPGLQYRTVSQTGPVHAP | |
| | VFAVAVEVNGLTFEGTGPTKKKAKMRAAELALRSFVQFPNACQAHLAMGG | |
| | GPGPGTDFTSDQADFPDTLFQEFEPPAPRPGLAGGRPGDAALLSAAYGRRRL | |
| | LCRALDLVGPTPATPAAPGERNPVVLLNRLRAGLRYVCLAEPAERRARSFV | |
| | MAVSVDGRTFEGSGRSKKLARGQAAQAALQELFDIQMPGHAPGRARRTPM | |
| | PQEFADSISQLVTQKFREVTTDLTPMHARHKALAGIVMTKGLDARQAQVVA | |
| | LSSGTKCISGEHLSDQGLVVNDCHAEVVARRAFLHFLYTQLELHLSKRREDS | |
| | ERSIFVRLKEGGYRLRENILFHLYVSTSPCGDARLHSPYEITTDLHSSKHLVR | |
| | KFRGHLRTKIESGEGTVPVRGPSAVQTWDGVLLGEQLITMSCTDKIARWNV | |
| | LGLQGALLSHFVEPVYLQSIVVGSLHHTGHLARVMSHRMEGVGQLPASYRH | |
| | NRPLLSGVSDAEARQPGKSPPFSMNWVVGSADLEIINATTGRRSCGGPSRLC | |
| | KHVLSARWARLYGRLSTRTPSPGDTPSMYCEAKLGAHTYQSVKQQLFKAFQ | |
| | KAGLGTWVRKPPEQQQFLLTL | |
| MCP | MASNFTQFVLVDNGGTGDVTVAPSNFANGIAEWISSNSRSQAYKVTCSVRQ | 7 |
| domain | SSAQNRKYTIKVEVPKGAWRSYLNMELTIPIFATNSDCELIVKAMQGLLKDG | |
| | NPIPSAIAANSGIY | |
| ADAR2DD | MQLHLPQVLADAVSRLVLGKFGDLTDNFSSPHARRKVLAGVVMTTGTDVK | 8 |
| (E488Q) | DAKVISVSTGTKCINGEYMSDRGLALNDCHAEIISRRSLLRFLYTQLELYLNN | |
| | KDDQKRSIFQKSERGGFRLKENVQFHLYISTSPCGDARIFSPHEPILEEPADRH | |
| | PNRKARGQLRTKIESGQGTIPVRSNASIQTWDGVLQGERLLTMSCSDKIARW | |
| | NVVGIQGSLLSIFVEPIYFSSIILGSLYHGDHLSRAMYQRISNIEDLPPLYTLNK | |
| | PLLSGISNAEARQPGKAPNFSVNWTVGDSAIEVINATTGKDELGRASRLCKH | |
| | ALYCRWMRVHGKVPSHLLRSKITKPNVYHESKLAAKEYQAAKARLFTAFIK | |
| | AGLGAWVEKPTEQDQFSLT | |
| MCP- | MASNFTQFVLVDNGGTGDVTVAPSNFANGIAEWISSNSRSQAYKVTCSVRQ | 9 |
| ADAR2DD | SSAQNRKYTIKVEVPKGAWRSYLNMELTIPIFATNSDCELIVKAMQGLLKDG | |
| (E488Q)- | NPIPSAIAANSGIYGGSGSGAGSGSPAGGGAPGSGGGSQLHLPQVLADAVSR | |
| NES | LVLGKFGDLTDNFSSPHARRKVLAGVVMTTGTDVKDAKVISVSTGTKCING | |
| | EYMSDRGLALNDCHAEIISRRSLLRFLYTQLELYLNNKDDQKRSIFQKSERG | |
| | GFRLKENVQFHLYISTSPCGDARIFSPHEPILEEPADRHPNRKARGQLRTKIES | |
| | GQGTIPVRSNASIQTWDGVLQGERLLTMSCSDKIARWNVVGIQGSLLSIFVEP | |
| | IYFSSIILGSLYHGDHLSRAMYQRISNIEDLPPLYTLNKPLLSGISNAEARQPGK | |
| | APNFSVNWTVGDSAIEVINATTGKDELGRASRLCKHALYCRWMRVHGKVP | |
| | SHLLRSKITKPNVYHESKLAAKEYQAAKARLFTAFIKAGLGAWVEKPTEQD | |
| | QFSLTGSGSGSLPPLERLTL | |
| P2A self- | ATNFSLLKQAGDVEENPGP | 10 |
| cleaving | | |
| peptide | | |
| T2A self- | EGRGSLLTCGDVEENPGP | 11 |
| cleaving | | |
| peptide | | |
| E2A self- | QCTNYALLKLAGDVESNPGP | 12 |
| cleaving | | |
| peptide | | |
| TagBFP | MSELIKENMHMKLYMEGTVDNHHFKCTSEGEGKPYEGTQTMRIKVVEGGP | 13 |
| | LPFAFDILATSFLYGSKTFINHTQGIPDFFKQSFPEGFTWERVTTYEDGGVLTA | |
| | TQDTSLQDGCLIYNVKIRGVNFTSNGPVMQKKTLGWEAFTETLYPADGGLE | |
| | GRNDMALKLVGGSHLIANIKTTYRSKKPAKNLKMPGVYYVDYRLERIKEAN | |
| | NETYVEQHEVAVARYCDLPSKLGH | |
| mNeonGreen | MVSKGEEDNMASLPATHELHIFGSINGVDFDMVGQGTGNPNDGYEELNLKS | 14 |
| | TKGDLQFSPWILVPHIGYGFHQYLPYPDGMSPFQAAMVDGSGYQVHRTMQF | |
| | EDGASLTVNYRYTYEGSHIKGEAQVKGTGFPADGPVMTNSLTAADWCRSK | |
| | KTYPNDKTIISTFKWSYTTGNGKRYRSTARTTYTFAKPMAANYLKNQPMYV | |
| | FRKTELKHSKTELNFKEWQKAFTDVMGMDELYK | |
| NanoLuc | MVFTLEDFVGDWRQTAGYNLDQVLEQGGVSSLFQNLGVSVTPIQRIVLSGE | 15 |
| luciferase | NGLKIDIHVIIPYEGLSGDQMGQIEKIFKVVYPVDDHHFKVILHYGTLVIDGV | |
| | TPNMIDYFGRPYEGIAVFDGKKITVTGTLWNGNKIIDERLINPDGSLLFRVTIN | |
| | GVTGWRLCERILA | |

TABLE 3
DNA sequences coding for target RNAs
(starting at initiation ATG codon)

| | | SEQ |
|---|---|---|
| Sequence | | ID |
| Type | Sequence | NO: |
| iRFP720, | ATGGCGGAAGGATCCGTCGCCAGGCAGCCTGACCTCTTGACCTGCGACGATG | 16 |
| cytosolic | AGCCGATCCATATCCCCGGTGCCATCCAACCGCATGGACTGCTGCTCGCCCT | |
| | CGCCGCCGACATGACGATCGTTGCCGGCAGCGACAACCTTCCCGAACTCACC | |
| | GGACTGGCGATCGGCGCCCTGATCGGCCGCTCTGCGGCCGATGTCTTCGACT | |
| | CGGAGACGCACAACCGTCTGACGATCGCCTTGGCCGAGCCCGGGGCGGCCG | |
| | TCGGAGCACCGATCACTGTCGGCTTCACGATGCGAAAGGACGCAGGCTTCAT | |
| | CGGCTCCTGGCATCGCCATGATCAGCTCATCTTCCTCGAGCTCGAGCCTCCCC | |
| | AGCGGGACGTCGCCGAGCCGCAGGCGTTCTTCCGCCGCACCAACAGCGCCAT | |
| | CCGCCGCCTGCAGGCCGCCGAAACCTTGGAAAGCGCCTGCGCCGCCGCGGC | |
| | GCAAGAGGTGCGGAAGATTACCGGCTTCGATCGGGTGATGATCTATCGCTTC | |
| | GCCTCCGACTTCAGCGGGTCCGTGATCGCAGAGGATCGGTGCGCCGAGGTCG | |
| | AGTCAAAACTAGGCCTGCACTATCCTGCCTCATTCATCCCGGCGCAGGCCCG | |
| | TCGGCTCTATACCATCAACCCGGTACGGATCATTCCCGATATCAATTATCGGC | |
| | CGGTGCCGGTCACCCCAGACCTCAATCCGGTCACCGGGCGGCCGATTGATCT | |
| | TAGCTTCGCCATCCTGCGCAGCGTCTCGCCCAACCATCTGGAGTTCATGCGC | |
| | AACATAGGCATGCACGGCACGATGTCGATCTCGATTTTGCGCGGCGAGCGAC | |
| | TGTGGGGATTGATCGTTTGCCATCACCGAACGCCGTACTACGTCGATCTCGA | |
| | TGGCCGCCAAGCCTGCGAGCTAGTCGCCCAGGTTCTGGCCTGGCAGATCGGC | |
| | GTGATGGAAGAGTAA | |
| iRFP720, | ATGCGGAAGGATCCGTCGCCAGGCAGCCTGACCTCTTGACCTGCGACGATGA | 17 |
| frame- | GCCGATCCATATCCCCGGTGCCATCCAACCGCATGGACTGCTGCTCGCCCTC | |
| shifted | GCCGCCGACATGACGATCGTTGCCGGCAGCGACAACCTTCCCGAACTCACCG | |
| | GACTGGCGATCGGCGCCCTGATCGGCCGCTCTGCGGCCGATGTCTTCGACTC | |
| | GGAGACGCACAACCGTCTGACGATCGCCTTGGCCGAGCCCGGGGCGGCCGT | |
| | CGGAGCACCGATCACTGTCGGCTTCACGATGCGAAAGGACGCAGGCTTCATC | |
| | GGCTCCTGGCATCGCCATGATCAGCTCATCTTCCTCGAGCTCGAGCCTCCCCA | |
| | GCGGGACGTCGCCGAGCCGCAGGCGTTCTTCCGCCGCACCAACAGCGCCATC | |
| | CGCCGCCTGCAGGCCGCCGAAACCTTGGAAAGCGCCTGCGCCGCCGCGGCG | |
| | CAAGAGGTGCGGAAGATTACCGGCTTCGATCGGGTGATGATCTATCGCTTCG | |
| | CCTCCGACTTCAGCGGGTCCGTGATCGCAGAGGATCGGTGCGCCGAGGTCGA | |
| | GTCAAAACTAGGCCTGCACTATCCTGCCTCATTCATCCCGGCGCAGGCCCGT | |
| | CGGCTCTATACCATCAACCCGGTACGGATCATTCCCGATATCAATTATCGGC | |
| | CGGTGCCGGTCACCCCAGACCTCAATCCGGTCACCGGGCGGCCGATTGATCT | |
| | TAGCTTCGCCATCCTGCGCAGCGTCTCGCCCAACCATCTGGAGTTCATGCGC | |
| | AACATAGGCATGCACGGCACGATGTCGATCTCGATTTTGCGCGGCGAGCGAC | |
| | TGTGGGGATTGATCGTTTGCCATCACCGAACGCCGTACTACGTCGATCTCGA | |
| | TGGCCGCCAAGCCTGCGAGCTAGTCGCCCAGGTTCTGGCCTGGCAGATCGGC | |
| | GTGATGGAAGAGTAA | |
| iRFP720, | ATGGGCGTGAAGGTCCTCTTCGCACTCATTTGCATAGCCGTAGCAGAAGCCG | 18 |
| secreted | ACTATAAAGACGACGATGACAAGGGCGGGTCCGGCGGAGCGGAAGGATCCG | |
| | TCGCCAGGCAGCCTGACCTCTTGACCTGCGACGATGAGCCGATCCATATCCC | |
| | CGGTGCCATCCAACCGCATGGACTGCTGCTCGCCCTCGCCGCCGACATGACG | |
| | ATCGTTGCCGGCAGCGACAACCTTCCCGAACTCACCGGACTGGCGATCGGCG | |
| | CCCTGATCGGCCGCTCTGCGGCCGATGTCTTCGACTCGGAGACGCACAACCG | |
| | TCTGACGATCGCCTTGGCCGAGCCCGGGGCGGCCGTCGGAGCACCGATCACT | |
| | GTCGGCTTCACGATGCGAAAGGACGCAGGCTTCATCGGCTCCTGGCATCGCC | |
| | ATGATCAGCTCATCTTCCTCGAGCTCGAGCCTCCCCAGCGGGACGTCGCCGA | |
| | GCCGCAGGCGTTCTTCCGCCGCACCAACAGCGCCATCCGCCGCCTGCAGGCC | |
| | GCCGAAACCTTGGAAAGCGCCTGCGCCGCCGCGGCGCAAGAGGTGCGGAAG | |
| | ATTACCGGCTTCGATCGGGTGATGATCTATCGCTTCGCCTCCGACTTCAGCGG | |
| | GTCCGTGATCGCAGAGGATCGGTGCGCCGAGGTCGAGTCAAAACTAGGCCT | |
| | GCACTATCCTGCCTCATTCATCCCGGCGCAGGCCCGTCGGCTCTATACCATCA | |
| | ACCCGGTACGGATCATTCCCGATATCAATTATCGGCCGGTGCCGGTCACCCC | |
| | AGACCTCAATCCGGTCACCGGGCGGCCGATTGATCTTAGCTTCGCCATCCTG | |
| | CGCAGCGTCTCGCCCAACCATCTGGAGTTCATGCGCAACATAGGCATGCACG | |
| | GCACGATGTCGATCTCGATTTTGCGCGGCGAGCGACTGTGGGGATTGATCGT | |
| | TTGCCATCACCGAACGCCGTACTACGTCGATCTCGATGGCCGCCAAGCCTGC | |
| | GAGCTAGTCGCCCAGGTTCTGGCCTGGCAGATCGGCGTGATGGAAGAGTAA | |
| human | ATGGAGGAGCCGCAGTCAGATCCTAGCGTCGAGCCCCCTCTGAGTCAGGAA | 19 |
| TP53 wild- | ACATTTTCAGACCTATGGAAACTACTTCCTGAAAACAACGTTCTGTCCCCCTT | |
| type | GCCGTCCCAAGCAATGGATGATTTGATGCTGTCCCCGGACGATATTGAACAA | |
| | TGGTTCACTGAAGACCCAGGTCCAGATGAAGCTCCCAGAATGCCAGAGGCTG | |
| | CTCCCCGCGTGGCCCCTGCACCAGCAGCTCCTACACCGGCGGCCCCTGCACC | |
| | AGCCCCCTCCTGGCCCCTGTCATCTTCTGTCCCTTCCCAGAAAACCTACCAGG | |
| | GCAGCTACGGTTTCCGTCTGGGCTTCTTGCATTCTGGGACAGCCAAGTCTGTG | |
| | ACTTGCACGTACTCCCCTGCCCTCAACAAGATGTTTTGCCAACTGGCCAAGA | |
| | CCTGCCCTGTGCAGCTGTGGGTTGATTCCACACCCCCGCCCGGCACCCGCGT | |
| | CCGCGCCATGGCCATCTACAAGCAGTCACAGCACATGACGGAGGTTGTGAG | |
| | GCGCTGCCCCCACCATGAGCGCTGCTCAGATAGCGATGGTCTGGCCCCTCCT | |
| | CAGCATCTTATCCGAGTGGAAGGAAATTTGCGTGTGGAGTATTTGGATGACA | |
| | GAAACACTTTTCGACATAGTGTGGTGGTGCCCTATGAGCCGCCTGAGGTTGG | |
| | CTCTGACTGTACCACCATCCACTACAACTACATGTGTAACAGTTCCTGCATGG | |
| | GCGGCATGAACCGGAGGCCCATCCTCACCATCATCACACTGGAAGACTCCAG | |
| | TGGTAATCTACTGGGACGGAACAGCTTTGAGGTGCGTGTTTGTGCCTGTCCT | |
| | GGGAGAGACCGGCGCACAGAGGAAGAGAATCTCCGCAAGAAAGGGGAGCC | |
| | TCACCACGAGCTGCCCCCAGGGAGCACTAAGCGAGCACTGCCCAACAACAC | |
| | CAGCTCCTCTCCCCAGCCAAAGAAGAAACCACTGGATGGAGAATATTTCACC | |
| | CTTCAGATCCGTGGGCGTGAGCGCTTCGAGATGTTCCGAGAGCTGAATGAGG | |
| | CCTTGGAACTCAAGGATGCCCAGGCTGGGAAGGAGCCAGGGGGGAGCAGGG | |
| | CTCACTCCAGCCACCTGAAGTCCAAAAAGGGTCAGTCTACCTCCCGCCATAA | |
| | AAAACTCATGTTCAAGACAGAAGGGCCTGACTCAGACTAG | |
| human | ATGGAGGAGCCGCAGTCAGATCCTAGCGTCGAGCCCCCTCTGAGTCAGGAA | 20 |
| TP53- | ACATTTTCAGACCTATGGAAACTACTTCCTGAAAACAACGTTCTGTCCCCCTT | |
| Y220H | GCCGTCCCAAGCAATGGATGATTTGATGCTGTCCCCGGACGATATTGAACAA | |
| | TGGTTCACTGAAGACCCAGGTCCAGATGAAGCTCCCAGAATGCCAGAGGCTG | |
| | CTCCCCGCGTGGCCCCTGCACCAGCAGCTCCTACACCGGCGGCCCCTGCACC | |
| | AGCCCCCTCCTGGCCCCTGTCATCTTCTGTCCCTTCCCAGAAAACCTACCAGG | |
| | GCAGCTACGGTTTCCGTCTGGGCTTCTTGCATTCTGGGACAGCCAAGTCTGTG | |
| | ACTTGCACGTACTCCCCTGCCCTCAACAAGATGTTTTGCCAACTGGCCAAGA | |
| | CCTGCCCTGTGCAGCTGTGGGTTGATTCCACACCCCCGCCCGGCACCCGCGT | |
| | CCGCGCCATGGCCATCTACAAGCAGTCACAGCACATGACGGAGGTTGTGAG | |
| | GCGCTGCCCCCACCATGAGCGCTGCTCAGATAGCGATGGTCTGGCCCCTCCT | |
| | CAGCATCTTATCCGAGTGGAAGGAAATTTGCGTGTGGAGTATTTGGATGACA | |
| | GAAACACTTTTCGACATAGTGTGGTGGTGCCCCATGAGCCGCCTGAGGTTGG | |
| | CTCTGACTGTACCACCATCCACTACAACTACATGTGTAACAGTTCCTGCATGG | |
| | GCGGCATGAACCGGAGGCCCATCCTCACCATCATCACACTGGAAGACTCCAG | |
| | TGGTAATCTACTGGGACGGAACAGCTTTGAGGTGCGTGTTTGTGCCTGTCCT | |
| | GGGAGAGACCGGCGCACAGAGGAAGAGAATCTCCGCAAGAAAGGGGAGCC | |
| | TCACCACGAGCTGCCCCCAGGGAGCACTAAGCGAGCACTGCCCAACAACAC | |
| | CAGCTCCTCTCCCCAGCCAAAGAAGAAACCACTGGATGGAGAATATTTCACC | |
| | CTTCAGATCCGTGGGCGTGAGCGCTTCGAGATGTTCCGAGAGCTGAATGAGG | |
| | CCTTGGAACTCAAGGATGCCCAGGCTGGGAAGGAGCCAGGGGGGAGCAGGG | |
| | CTCACTCCAGCCACCTGAAGTCCAAAAAGGGTCAGTCTACCTCCCGCCATAA | |
| | AAAACTCATGTTCAAGACAGAAGGGCCTGACTCAGACTAG | |

TABLE 4
DNA coding for sensor sequences (75 bp) targeting
iRFP720 mRNAs, recruiting wild-type ADARs

| | | SEQ |
|---|---|---|
| Sequence | | ID |
| Type | Sequence | NO: |
| CCA60 with | AGCAGCAGTCCATGCGGTTGGA | 21 |
| single stop | TTGCACCGGGGATATAGATCGG | |
| codon | CTCATCGTCGCAGGTCAAGAGG | |
| | TCAGGCTGC | |
| CCA60 with | AGCAGCAGTCCATGCGGTTAGA | 22 |
| multiple stop | TTGCACCGGGGATATAGATCGG | |
| codons | CTCATCGTCGCAGGTCAAGAGG | |
| | TCAGGCTGC | |
| CCA74 with | CGGCGGCGAGGGCGAGCAGCAG | 23 |
| single stop | TCCATGCGGTTGGATAGCACCG | |
| codon | GGGATATGGATCGGCTCATCGT | |
| | CGCAGGTCA | |
| CCA78 with | ATGTCGGCGGCGAGGGCGAGCA | 24 |
| single stop | GCAGTCCATGCGGTTAGATTGC | |
| codon | ACCGGGGATATGGATCGGCTCA | |
| | TCGTCGCAG | |
| CCA78 with | ATGTCGGCGGCGAGGGCGAGCA | 25 |
| multiple stop | GCAGTCCATGCGGTTAGATTGC | |
| codons | ACCGGGGATATAGATCGGCTCA | |
| | TCGTCGCAG | |
| CCA413 with | CGCTTTCCAAGGTTTCGGCGGC | 26 |
| single stop | CTGCAGGCGGCGGATAGCGCTG | |
| codon | TTGGTGCGGCGGAAGAACGCCT | |
| | GCGGCTCGG | |
| CCA413 with | CGCTTTCCAAGGTTTCGGCGGC | 27 |
| multiple stop | CTGCAGGCGGCGGATAGCGCTG | |
| codons | TTAGTGCGGCGGAAGAACGCCT | |
| | GCGGCTCGG | |
| CCA762 with | GTGCCGTGCATTCCTATTTTGC | 28 |
| single stop | GCATTAACTCCAGATAGTTGGG | |
| codon | CGAGACGCTGCGCAGGATTGCG | |
| | AAGCTAAGA | |

TABLE 5
DNA coding for sensor sequences targeting
iRFP720 mRNAs, recruiting MCP fusions

| | | SEQ |
|---|---|---|
| Sequence | | ID |
| Type | Sequence | NO: |
| CCA60, | GCGGCGAGGGCGAGCAGACATGAGGATCA | 29 |
| equidistant | CCCATGTTGCGGTTGGATTGCACCGGGGA | |
| MS2 hairpins | TATAGATCGGCTCATCGTCGCAGGTCAAA | |
| | CATGAGGATCACCCATGTGGCTGCCTGGC | |
| | GACGGAT | |
| CCA60, 5′- | GCGGCGAGGGCGAGACATGAGGATCACCC | 30 |
| shifted MS2 | ATGTCCATGCGGTTGGATTGCACCGGGGA | |
| hairpins | TATAGATCGGCTCATCGTCGCAGGTACAT | |
| | GAGGATCACCCATGTTCAGGCTGCCTGGC | |
| | GACGGAT | |
| CCA60, 3′- | GCGGCGAGGGCGAGCAGCAGACATGAGGA | 31 |
| shifted MS2 | TCACCCATGTGGTTGGATTGCACCGGGGA | |
| hairpins | TATAGATCGGCTCATCGTCGCAGGTCAAG | |
| | AGACATGAGGATCACCCATGTTGCCTGGC | |
| | GACGGAT | |
| CCA74, | CGATCGTCATGTCGGCGACATGAGGATCA | 32 |
| equidistant | CCCATGTCGAGCAGCAGTCCATGCGGTTG | |
| MS2 hairpins | GATAGCACCGGGGATATGGATCGGCTCAA | |
| | CATGAGGATCACCCATGTAGGTCAAGAGG | |
| | TCAGGCT | |
| CCA74, 5′- | CGATCGTCATGTCGACATGAGGATCACCC | 33 |
| shifted MS2 | ATGTGGGCGAGCAGCAGTCCATGCGGTTG | |
| hairpins | GATAGCACCGGGGATATGGATCGGCACAT | |
| | GAGGATCACCCATGTCGCAGGTCAAGAGG | |
| | TCAGGCT | |
| CCA74, 3′- | CGATCGTCATGTCGGCGGCGACATGAGGA | 34 |
| shifted MS2 | TCACCCATGTGCAGCAGTCCATGCGGTTG | |
| hairpins | GATAGCACCGGGGATATGGATCGGCTCAT | |
| | CGACATGAGGATCACCCATGTTCAAGAGG | |
| | TCAGGCT | |
| CCA78, | GCAACGATCGTCATTTCACATGAGGATCA | 35 |
| equidistant | CCCATGTAGGGCGAGCAGCAGTCCATGCG | |
| MS2 hairpins | GTTAGATTGCACCGGGGATATGGATCGGA | |
| | CATGAGGATCACCCATGTTCGCAGGTCAA | |
| | GAGGTCA | |
| CCA78, 5′- | GCAACGATCGTCATACATGAGGATCACCC | 36 |
| shifted MS2 | ATGTGCGAGGGCGAGCAGCAGTCCATGCG | |
| hairpins | GTTAGATTGCACCGGGGATATGGATACAT | |
| | GAGGATCACCCATGTTCGTCGCAGGTCAA | |
| | GAGGTCA | |
| CCA78, 3′- | GCAACGATCGTCATTTCGGCACATGAGGA | 37 |
| shifted MS2 | TCACCCATGTGCGAGCAGCAGTCCATGCG | |
| hairpins | GTTAGATTGCACCGGGGATATGGATCGGC | |
| | TCACATGAGGATCACCCATGTCAGGTCAA | |
| | GAGGTCA | |

TABLE 6
DNA coding for sensor sequences targeting
human p53 mRNAs, recruiting MCP fusions

| | | SEQ |
|---|---|---|
| Sequence | | ID |
| Type | Sequence | NO: |
| Sensor | CAGTTGCAGTGGATTGTACATGAG | 38 |
| differentiating | GATCACCCATGTTCAGAGCCAACC | |
| wild-type | TCAGGCGGCTCATAGGGCACCACC | |
| and Y220H | ACACTATGTCGAAAACATGAGGAT | |
| mutant p53 | CACCCATGTCTGTCATCCAAATAC | |
| | TCC | |

TABLE 7
DNA coding for sensor sequences targeting
murine mRNAs, recruiting MCP fusions

| | | SEQ |
|---|---|---|
| Sequence | | ID |
| Type | Sequence | NO: |
| Sensor | CCACTTAAAAGCCCCCTACATGAGGATCAC | 39 |
| targeting | CCATGTAAGGGATGGCTTTTGACACCAACT | |
| myogenin | TAGGGGCTCACATGCACACCCAGCCTACAT | |
| mRNAs at | GAGGATCACCCATGTAATCTCAGTTGGGCA | |
| position | TGG | |
| CCA1843 | | |
| Sensor | GAAGCTCCTGAGTTTGCACATGAGGATCAC | 40 |
| targeting | CCATGTAGGGACATTAACAAGGGGGCTCTC | |
| myogenin | TAGACTCCATCTTTCTCTCCTCAGAAACAT | |
| mRNAs at | GAGGATCACCCATGTCTCTCTGCTTTAAGG | |
| position | AGT | |
| CCA2046 | | |
| Sensor | AGCTCCTCCCCCTTCTCACATGAGGATCAC | 41 |
| targeting | CCATGTTCAGGGCAGGCCCAGCCCAGCCAC | |
| myogenin | TAGCTTCAGGAAGAGACTAGAACAGAACAT | |
| mRNAs at | GAGGATCACCCATGTACTTGTCCAGGTCAG | |
| position | GGC | |
| CCA2217 | | |
| Sensor | GGCAGCTTTACAAACAAACATGAGGATCAC | 42 |
| targeting | CCATGTAACAATAAACAATACACAAAGCAC | |
| myogenin | TAGAAGGTTCCCAAGATCCACTGCAAACAT | |
| mRNAs at | GAGGATCACCCATGTGCCCCCAGAGGCTTT | |
| position | GGA | |
| CCA2323 | | |
| Sensor | CCCACCCCCTTCCCTGCACATGAGGATCAC | 43 |
| targeting | CCATGTCGGTATCATCAGCACAGGAGACCT | |
| myogenin | TAGTCAGACGGCAGCTTTACAAACAAACAT | |
| mRNAs at | GAGGATCACCCATGTAACAATAAACAATAC | |
| position | ACA | |
| CCA2380 | | |
| Sensor | AAAGGAAATCCAAATAAACATGAGGATCAC | 44 |
| targeting | CCATGTAAAGAATTACAAAAGAAAAAAAAT | |
| myogenin | TAGCAAAACCACACAATTCTTAGTTCACAT | |
| mRNAs at | GAGGATCACCCATGTAAGTCACCCCAAGAG | |
| position | CCC | |
| CCA2478 | | |
| Sensor | ACAAAGGGGAATTTGTCACATGAGGATCAC | 45 |
| targeting | CCATGTAGCCGGGTCTCCTCGCCCGTGTTG | |
| alkaline | TAGTGTAGCTGGCCCTTAAGGATTCGACAT | |
| phosphatase | GAGGATCACCCATGTGTTACTGTGGAGACG | |
| mRNAs at | CCC | |
| position | | |
| CCA41237 | | |
| Sensor | TGGATTGGACCTCATGGACATGAGGATCAC | 46 |
| targeting | CCATGTTGGTGTTGCATCGCGTGCGCTCTG | |
| alkaline | TAGCTGCGCTCACTCCCACTGTGCCCACAT | |
| phosphatase | GAGGATCACCCATGTCCTTCACGCCACACA | |
| mRNAs at | AGT | |
| position | | |
| CCA42443 | | |
| Sensor | AGCTCTTCCAAATACGGACATGAGGATCAC | 47 |
| targeting | CCATGTCCAGGCCATCTAGCCTTGTACCCC | |
| alkaline | TAGCCTTCTCATCCAGTTCGTATTCCACAT | |
| phosphatase | GAGGATCACCCATGTTTCTGTTCTTCGGGT | |
| mRNAs at | ACA | |
| position | | |
| CCA46769 | | |

TABLE 8
List of Oligonucleotides

| | | SEQ |
|---|---|---|
| | | ID |
| Sequence Type | Sequence | NO: |
| Oligonucleotide 1 | CCGGAGGCCCTAGATCTTCTTGAC | 48 |
| Oligonucleotide 2 | GGGACTGCTCCTTCACCACC | 49 |
| Oligonucleotide 3 | TTGCTCAGCTCCCTCAACCAG | 50 |
| Oligonucleotide 4 | AGCCGCGAGCAAATGATCTC | 51 |
| Oligonucleotide 5 | GGCGCATCAAGGAGCTCACC | 52 |
| Oligonucleotide 6 | CCTGCTCCTCCGCCTCCTC | 53 |
| Oligonucleotide 7 | CACCTGCCTTACCAACTCTTTTGTG | 54 |
| Oligonucleotide 8 | GGCTACATTGGTGTTGAGCTTTTGG | 55 |
| Oligonucleotide 9 | TTAAGGGCCTGCAGGGTG | 56 |
| Oligonucleotide 10 | TGGCTAGCCCCTCGAGT | 57 |
| Oligonucleotide 11 | CTGAAACAGGCAGGAGATGTGGA | 58 |
| Oligonucleotide 12 | CCGCATGTAAGCAGACTTCCTCT | 59 |
| Oligonucleotide 13 | TGCTCTCCTGTTGTGCTTCTCC | 60 |
| Oligonucleotide 14 | AGCCTCCCATTCAATTGCCAC | 61 |
| Oligonucleotide 15 | AAGAAGAAAAAAGCATCTGAGCCTGG | 62 |
| Oligonucleotide 16 | AGCCTCCCATTCAATTGCCAC | 63 |
| Oligonucleotide 17 | ACGACCACTTTGTCAAGCTCATTTC | 64 |
| Oligonucleotide 18 | GCAGTGAGGGTCTCTCTCTTCCTCT | 65 |

### Results:

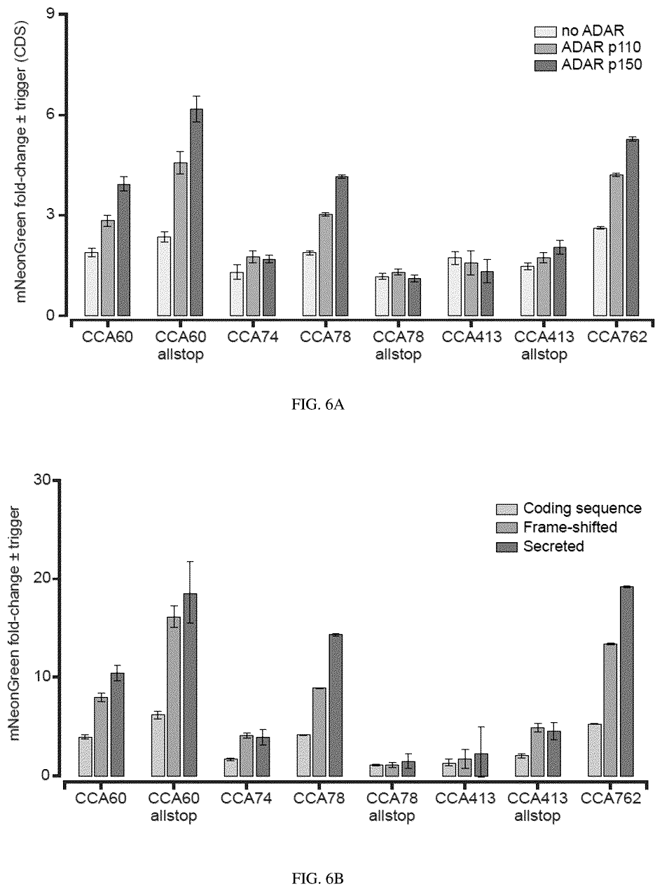

Performance of ADAR-based riboregulators is dependent on enzyme availability: The hypothesis that ADAR availability is a limiting factor in RNA editing-based sensors was tested. To do so, a basic ADAR-mediated RNA-responsive sensor architecture was designed and tested. Flow cytometry analysis was used to quantify the expression of the mNeonGreen detectable protein across a panel of sensors targeting different regions of a trigger nucleic acid encoding the iRFP720 fluorescent protein (see, FIGS. 5A-5C). The ADAR-mediated RNA-responsive sensors were designed to be complementary to sequences centered on CCA sites in the trigger nucleic acid, with the exception of the adenosine in the premature stop codon (see, FIG. 4). Co-transfection of plasmids encoding the ADAR-mediated RNA-responsive sensor variants and trigger nucleic acid in HEK293FT cells consistently resulted in higher detectable protein expression compared to the ADAR-mediated RNA-responsive sensor alone, across all CCA sites tested. This trend was observed for both short (51 bp) and longer (75 bp, see, FIGS. 6A-6B) sensor sequences; as the latter provided better performance, the experiments proceeded with 75 bp sensor sequences for optimization of the basic ADAR-mediated sensor designs. In agreement with the hypothesis, supplying exogenous ADAR resulted in a marked increase in mNeonGreen output levels, which suggested that low endogenous levels of ADAR limit ADAR-mediated RNA-responsive sensor performance (see, FIG. 1C). Of note, the p150 isoform of ADAR1 seemed to enhance output expression more than the p110 isoform, yielding up to 9-fold activation.

The output activation in cells transfected with an ADAR-mediated RNA-responsive sensor, ADAR p150, and a trigger nucleic acid was readily observable at the protein level via microscopy, whereas this was not the case for cells transfected with the ADAR-mediated RNA-responsive sensor and trigger nucleic acid alone (see, FIG. 1D). To quantify this output at the mRNA level, the editing efficiency of ADAR-mediated RNA-responsive sensor transcripts was evaluated via next-generation sequencing (see, FIG. 1E). Over 30% editing was observed of the adenosine in the premature UAG stop codon of ADAR-mediated RNA-responsive sensor transcripts harvested from cells transfected with an ADAR-mediated RNA-responsive sensor, ADAR p150, and trigger nucleic acid. Importantly, this editing was observed to a much lesser extent (about 3%) in cells receiving only the ADAR-mediated RNA-responsive sensor and trigger nucleic acid plasmids, confirming that endogenous ADAR edits ADAR-mediated RNA-responsive sensor transcripts but that its activity is insufficient to mediate efficient detection of trigger nucleic acids. Additionally, it was found that ADAR-mediated editing is specific: substantial off-target editing of other nearby adenosine residues in the ADAR-mediated RNA-responsive sensor was not detected. These data, combined with considerations about deployment of such a technology for practical applications, prompted engineering of ADAR-mediated RNA-responsive sensors containing an autocatalytic feedback motif that does not require constitutive ADAR expression for sensitive detection of trigger nucleic acids.

A self-amplifying circuit was engineered that consists of an ADAR-mediated RNA-responsive sensor transcript containing four in-frame components insulated by self-cleaving 2A peptides (see, FIG. 1F). A transfection marker serving as a reporter (TagBFP) was cloned upstream of the premature UAG stop codon in the sensor sequence to normalize for plasmid dosage. The ADAR-mediated RNA-responsive sensor comprised a sequence of sufficient complementarity to a trigger nucleic acid, with the exception of the adenosine in the premature UAG stop codon. The conditionally expressed detectable protein (mNeonGreen) was encoded downstream of this premature stop codon. Further, an ADAR coding sequence was linked to the ADAR-mediated RNA-responsive sensor output via another 2A peptide. In this system, it was expected that all cells transcribing the ADAR-mediated RNA-responsive sensors would produce the TagBFP reporter, but only cells expressing the trigger nucleic acid would also produce mNeonGreen detectable protein and exogenous ADAR encoded within the ADAR-mediated RNA-responsive sensor.

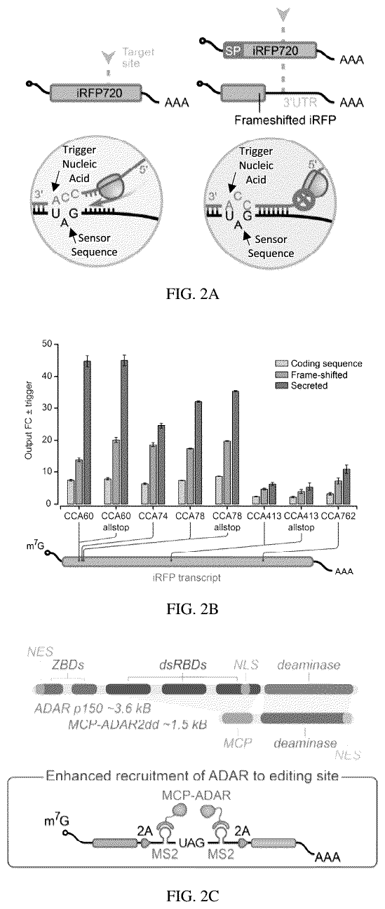

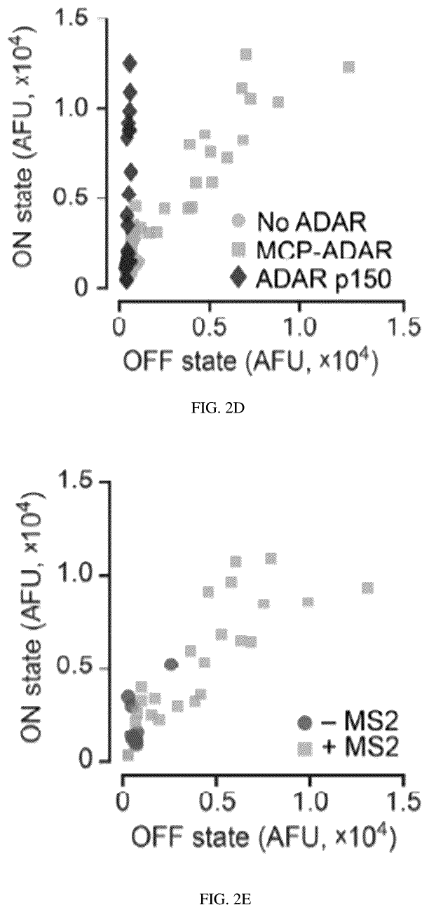

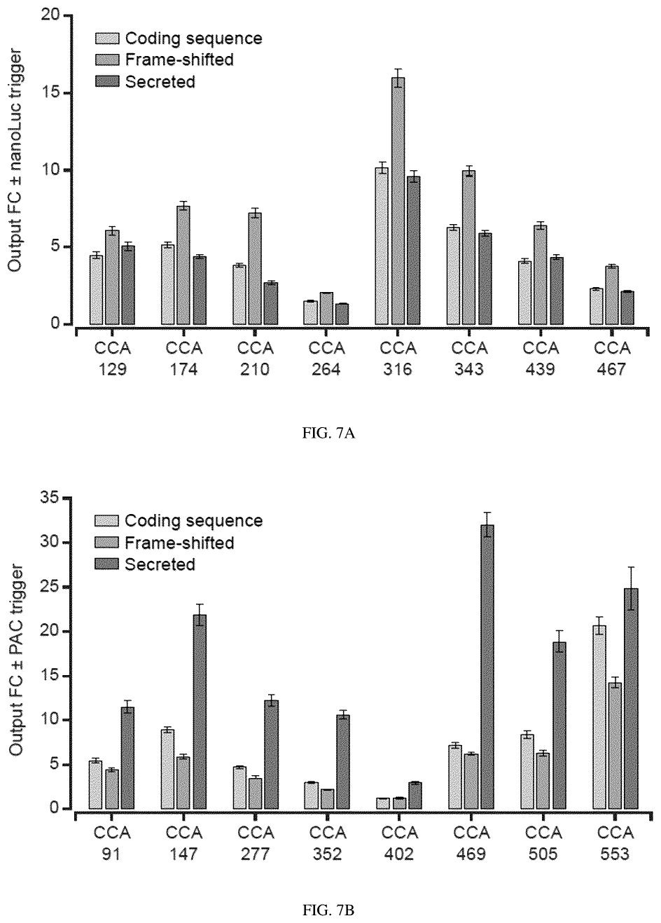

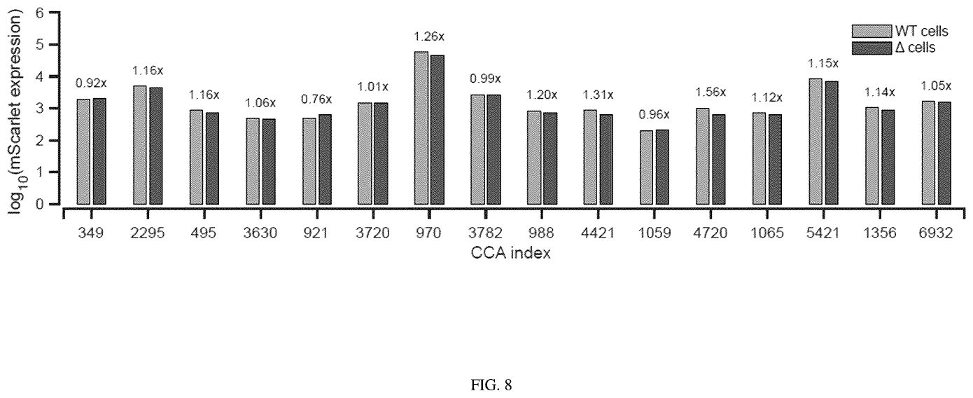

Identification of design rules for ADAR-mediated RNA-responsive sensors: Considering the modular nature of the ADAR-mediated RNA-responsive sensors, the trigger nucleic acid-sensor sequence interface was independently optimized starting with a simple topology in which ADAR was expressed constitutively from a separate transcript rather that conditionally from the ADAR-mediated RNA-responsive sensor transcript. General rules for the efficient targeting of trigger nucleic acids were defined. It was reasoned that since most gene-length RNA sequences harbor multiple CCA motifs, the question of which target sites within the trigger nucleic acid should be prioritized for ADAR-mediated RNA-responsive sensor engineering needed to be addressed. It was hypothesized that the translational machinery may interfere with ADAR editing by disrupting dsRNA in coding sequences (see, FIG. 2A) (15). To test this, the performance of the validated ADAR-mediated RNA-responsive sensors targeting trigger nucleic acid sequences were compared in three different contexts: (a) within the original protein coding sequence; (b) in a frame-shifted construct such that the target sites are part of the mRNA 3′UTR; and (c) in the coding sequence of a secreted version of the same protein (see, FIG. 2A). Across all ADAR-mediated RNA-responsive sensor sequences, targeting a secreted protein (and to a lesser extent a 3′UTR) yielded much higher activation levels than the same sites within the original protein-coding sequence—up to about 45-fold (see, FIG. 2B and FIGS. 7A-7B). Since, during the translation of a secreted protein, the ribosome translates the beginning of the protein, then pauses until the protein is inserted in the reticulum membrane (after which translation resumes) (16), the observations suggested that the pause limits the number of ribosomes scanning the coding sequence and potentially displacing the RNA:RNA duplexes that used for sensing and that ribosome-free RNA sequences were generally better trigger nucleic acids. This mechanistic insight explained why preliminary descriptions of ADAR-based sensors reported highly variable performance depending on the chosen target RNA (11). Of note, the importance of ribosome occupancy strongly suggests that ADAR-mediated editing was cytoplasmic. To probe this further, sensors were designed against the nuclear transcript MALAT1 (17). Output activation was not observed even when the transfection was supplemented with the predominantly nuclear-localized p110 isoform of ADAR (18) (see, FIG. 8). This supported and reinforced the hypothesis that in the system most editing events take place in the cytosol.

Next, the design of the modules that mediate autocatalysis in ADAR-mediated RNA-responsive sensors was optimized. The natural ADAR isoforms are large (FIG. 2A), and therefore using one of these as the amplifier would undercut the delivery potential of constructs. For instance, clinically approved adeno-associated viruses (AAVs) have a packaging limit of about 5 kilobases (19); the coding sequence of ADAR p150 would therefore expend over 70% of that capacity. Additionally, the dsRNA binding domains that mediate the recruitment of natural ADARs are promiscuous; supplying one of these ADARs in trans could therefore carry risks of off-target effects in bystander transcripts. Drawing inspiration from prior work focused on RNA-guided endogenous transcript editing (20,21), these limitations were overcome by substituting natural ADARs with an engineered ADAR variant that (a) contains only the ADAR catalytic domain necessary for RNA editing, and (b) could be recruited to the edit site in the sensor sequence to increase the frequency of editing events. To this end, a hyperactive, minimal version of ADAR2, namely MCP-ADAR2DD(E488Q)-NES was used (see, FIG. 2C) (20)—hereafter referred to as MCP-ADAR. The MS2 bacteriophage major coat protein (MCP) specifically binds to a short MS2 RNA hairpin and replaces the promiscuous dsRNA-interacting domains of natural ADAR enzymes with a short, localized, and orthogonal RNA-binding moiety. MCP-ADAR was integrated in-frame in the sensor transcript and added two MS2 hairpins flanking the premature UAG stop codon in the sensor sequence.

Upon testing the activity of ADAR-mediated RNA-responsive sensors comprising MS2 hairpins in human cells, it was observed that the constitutive expression of MCP-ADAR resulted in high ADAR-mediated RNA-responsive sensor activation in the absence of trigger nucleic acid (see, FIG. 2D), thus reducing dynamic range. However, since leaky activation by MCP-ADAR was only observed in ADAR-mediated RNA-responsive sensors harboring MS2 hairpins (FIG. 2E), it was inferred that the basal activation by MCP-ADAR was unlikely to be indicative of promiscuous activity that would result in the editing of off-target transcripts. Therefore, it was reasoned that MCP-ADAR would be a viable option for the ADAR system if the dynamic range was enhanced by reducing background editing.

Compact autocatalytic architecture boosts sensor performance: In the ADAR-mediated RNA-responsive sensors (see, FIG. 3A), MCP-ADAR was expressed only upon ADAR-mediated RNA-responsive sensor activation. In the presence of trigger nucleic acid, these ADAR-mediated RNA-responsive sensors relied on an initial editing step by endogenous ADARs, thereby yielding stop-less transcripts from which MCP-ADAR can be translated. In turn, MCP-ADAR could edit additional ADAR-mediated RNA-responsive sensor molecules upon recruitment to the MS2 hairpins (integrated in the various ways shown in FIG. 3B), thus efficiently amplifying the initial signal. This formed a positive feedback loop in which edited ADAR-mediated RNA-responsive sensors gave rise to the enzyme that further catalyzed this editing. It was reasoned that this approach could capitalize on the low background editing by natural ADARs and the targeted and efficient editing by MCP-ADAR. Additionally, the system was highly compact and could be encoded in a single transcript, potentially facilitating its delivery to cells of interest.

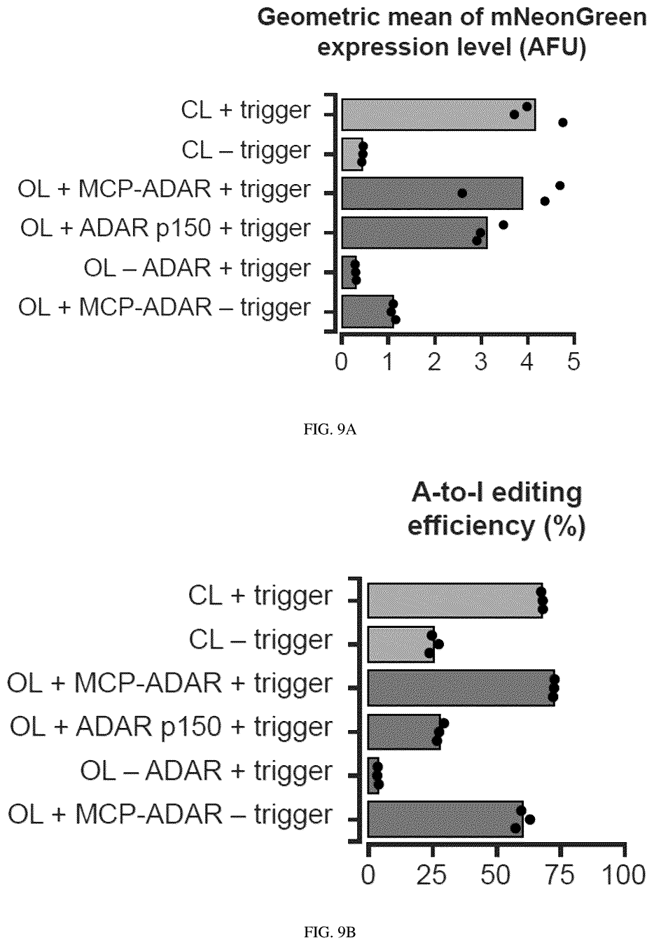

To benchmark the performance of the ADAR-mediated RNA-responsive sensor in terms of dynamic range at the protein level, its activity (closed loop, CL) was compared against an open-loop (OL) control in which MCP-ADAR was constitutively expressed in trans (see, FIG. 3C). It was observed that, across all tested prototypes, the ADAR-mediated RNA-responsive sensor yielded low background translational activation without compromising maximal activity in the presence of the trigger nucleic acid (see, FIGS. 3D-3E). NGS analysis confirmed that a marked decrease in A-to-I editing in the absence of trigger underlies the reduction in background observed by flow cytometry (FIGS. 9A-9B). As a result, the great majority of tested ADAR-mediated RNA-responsive sensor prototypes had increased dynamic range compared to the open-loop system, which suggested broad applicability of this approach for improving ADAR-mediated RNA-responsive sensor performance (see, FIGS. 3D-3E and 9A-9B). A poly-transfection was used to de-correlate the amounts of ADAR-mediated RNA-responsive sensors and trigger nucleic acid (22), highlighting that the implementation of the feedback mechanism improved the transfer function for a given amount of ADAR-mediated RNA-responsive sensor (see, FIG. 3F).

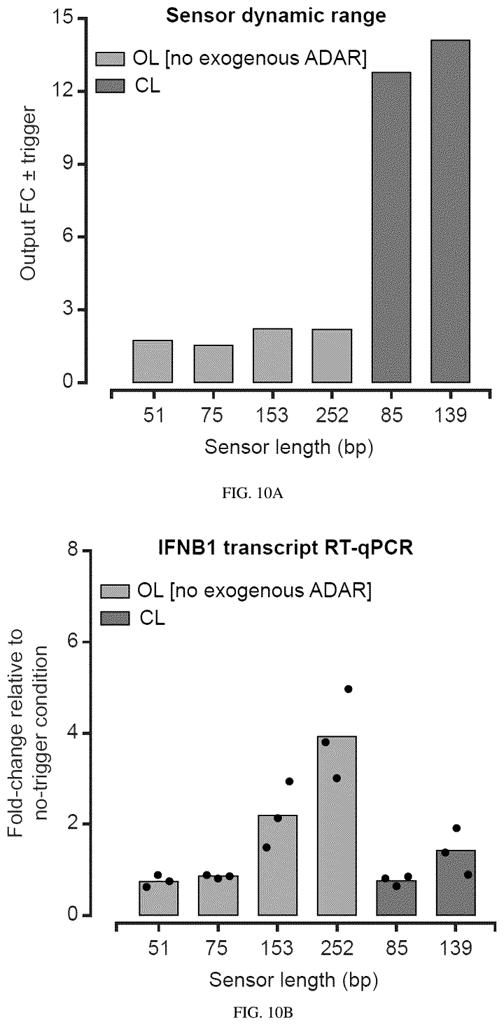

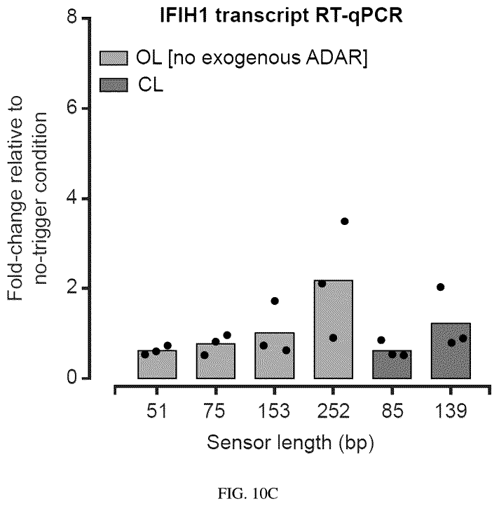

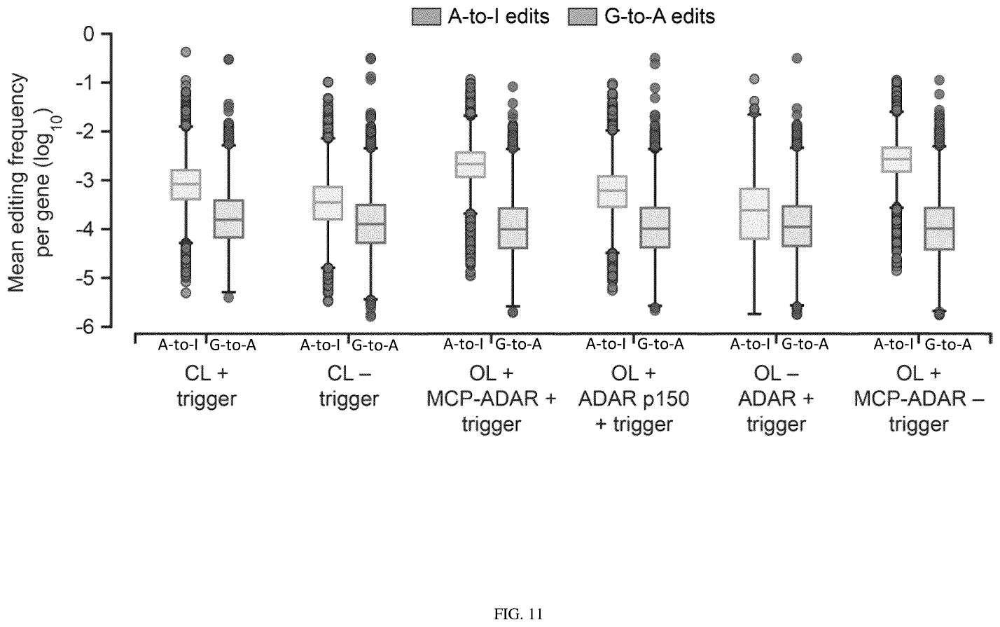

Additionally, the performance of ADAR-mediated RNA-responsive sensors relying on only endogenous ADAR was not appreciably improved with sensor length (see, FIGS. 10A10-C). Together, these results suggested that the ADAR-mediated RNA-responsive sensor architecture was a promising approach to generate useful in vivo sense-and-respond modules.

To further characterize the safety profiles of open- (no exogenous ADAR) and closed-loop architectures (featuring conditional expression of an MCP-ADAR), A-to-I editing was compared between these architectures. It was found that the frequency of off-target RNA editing was reduced in the closed-loop architecture relative to the open loop architecture (FIG. 11).

ADAR-mediated RNA-responsive sensors are specific and sensitive: To explore the utility of the ADAR-mediated RNA-responsive sensor cassettes for sensing cellular states, their specificity and sensitivity was tested in model mammalian cell lines. First, it was investigated whether ADAR editing could be leveraged to discriminate between two RNA molecules with minimal differences. Somatic mutations are responsible for myriad complex diseases, ranging from cancer to cardiovascular and neurological conditions (23,24). Therefore, the ability to discriminate healthy and diseased cells in mosaic tissues would be of great interest for precision therapeutics. Then, it was tested whether an ADAR-mediated RNA-responsive sensor targeted towards a point mutation of interest could specifically trigger translation in cells expressing a disease biomarker. As a case study, focus was directed to a single-base mutation in the human p53 tumor suppressor gene (c.658T>C), which results in a Y220H substitution that is known to destabilize the DNA binding domain of p53, making it a driver of breast, lung, and liver cancers (25, 26). HEK293FT cells were with an ADAR-mediated RNA-responsive sensor specifically designed to detect a trigger nucleic acid corresponding to the p53 mutant, alongside plasmids expressing either the wild-type or Y220H mutant p53 gene. The sensor was designed to be fully complementary to the Y220 codon and the surrounding sequence, such that the target adenosine could not be edited by ADAR; conversely, imperfect hybridization with the mutant RNA produced a single base-pair bulge, exposing the adenosine for editing by ADAR. A 5-fold activation was observed in the detectable protein gene downstream of the ADAR-mediated RNA-responsive sensor in cells expressing p53-Y220H, highlighting the specificity of the ADAR-mediated RNA-responsive sensors (see, FIG. 3G).

Next, it was investigated whether the ADAR-mediated RNA-responsive sensors could be used to discriminate closely related cell types based on deferentially expressed endogenous genes. Experiments were focused on the C2C12 murine myoblast cell line, a well-described model of cell differentiation (see, FIG. 3H). When they reach confluency, and particularly in serum-restricted conditions, C2C12 cells differentiate to form functional myotubes. Alternatively, upon exposure to bone morphogenetic protein-2 (BMP-2), the cells are biased to differentiate towards an osteoblastic lineage (27). ADAR-mediated RNA-responsive sensors were designed targeting trigger nucleic acid markers of both cell fates, namely the 3′ UTRs of mRNAs encoding myogenin and slow-twitch myosin heavy chain I (two proteins expressed during myogenesis), and the coding sequence of alkaline phosphatase (a bone-mineralizing enzyme). C2C12 cells were differentiated and confirmed phenotypically either by the presence of multinucleated syncytia indicative of early myotube formation (eventually forming functional contractile units), or by the detection of strong alkaline phosphatase activity (see, FIG. 3H). Reverse transcription followed by quantitative PCR (RT-qPCR) also confirmed the expected increase in the expression of the trigger nucleic acids corresponding to the target mRNAs (see, FIG. 3I), which indicated transcriptional changes that drive differentiation. It was observed that the ADAR-mediated RNA-responsive sensor constructs expressed the detectable protein (Nanoluc luciferase) as a response: ADAR-mediated RNA-responsive sensors targeting the myogenin and myosin mRNAs were activated in myotubes (see, FIG. 3J), while alkaline phosphatase-targeting ADAR-mediated RNA-responsive sensors were strongly activated in BMP-2-induced osteoblasts (see, FIG. 3K)—up to 80% of the maximum level defined by a control comprising no premature stop codon.

These observations demonstrated that the ADAR-mediated RNA-responsive sensor constructs were sensitive enough to drive high levels of expression of user-defined payloads in response to endogenous levels of trigger nucleic acids, making them well suited to sense and respond to transcriptional changes across both cell types and cell states.

### Conclusion:

The previously described constructs comprise a sensitive, programmable, modular, and compact RNA sense-and-respond circuit. Hybridization of a ADAR-mediated RNA-responsive sensor with a user-defined trigger nucleic acid transcript initiated RNA editing of a premature stop codon, driving the translation of the downstream output and detectable protein sequences. A secondary payload in the form of a hyperactive, minimal version of ADAR2 was validated and targeted to the edit site via the MS2 RNA hairpin-coat protein interaction, which resulted in an autocatalytic positive feedback loop. This configuration relied on endogenous ADAR to elicit the initial response with a high degree of specificity. It was demonstrated that by using autocatalysis, the circuit background was attenuated and the output dynamic range was enhanced by close to eight-fold relative to an open-loop configuration, while reducing the overall number of components and genetic footprint of the technology. The resulting circuit was able to detect minimal differences between RNA molecules and interpret endogenous signals to control transgene expression across different cell states.

While general rules for targeting user-defined RNA triggers were defined (see FIGS. 4 and 12), the choice of a target site for in vivo applications might involve additional considerations beyond editing efficiency.

Machine learning would be a straightforward way to optimize trigger nucleic acid detection, as has been done for toehold switches (28,29), but the design of ADAR-mediated RNA-responsive sensors might involve unique trade-offs between efficacy and safety. ADAR-mediated RNA-responsive sensor sequences encode translated peptides, the exact sequences of which are defined by the target RNA sites; different ADAR-mediated RNA-responsive sensors targeting a given RNA sequence will therefore produce different peptides, which might vary in their immunogenicity. We expect that recent computational advances in the prediction of peptide immunogenicity could be leveraged to further refine the prediction of optimal target sites (30), thereby guiding the design of therapeutically relevant ADAR-mediated RNA-responsive sensors.

These results expanded the application space of editing-based riboregulators: the autocatalytic feedback implementation featured a size of less than 5 kb (including promoter and terminator), making it amenable for delivery in clinically relevant vectors (19). Importantly, as ADAR enzymes are endogenously expressed in most human tissues (14), we expect most cells to be able to trigger autocatalysis when provided with sensors. It is envisioned that the sensors could lay the foundation for easy-to-deliver smart RNA-based therapeutics.

### REFERENCES

1. K Ilia, D Del Vecchio, Squaring a Circle: To What Extent Are Traditional Circuit Analogies Impeding Synthetic Biology? GEN Biotechnology 1, 150-155 (2022).
2. F Sedlmayer, D Aubel, M Fussenegger, Synthetic gene circuits for the detection, elimination and prevention of disease. Nature Biomedical Engineering 2, 399-415 (2018).
3. Tabula Sapiens Consortium, et al., The Tabula Sapiens: a multiple-organ, single-cell transcriptomic atlas of humans. Science (New York, N.Y.) 376, eab14896 (2022).
4. M A English, R V Gayet, J J Collins, Designing biological circuits: synthetic biology within the operon model and beyond. Annual Review of Biochemistry 90, 221-244 (2021).
5. C M Schmidt, C D Smolke, RNA switches for synthetic biology. Cold Spring Harbor Perspectives in Biology 11, a032532 (2019).
6. A A Green, P A Silver, J J Collins, P Yin, Toehold switches: de-novo-designed regulators of gene expression. Cell 159, 925-939 (2014).
7. K H Siu, W Chen, Riboregulated toehold-gated gRNA for programmable CRISPR-Cas9 function. Nature Chemical Biology 15, 217-220 (2018).
8. E M Zhao, et al., RNA-responsive elements for eukaryotic translational control. Nature Biotechnology 40, 539-545 (2021).
9. K E Kaseniit, et al., Modular and programmable RNA sensing using ADAR editing in living cells. bioRxiv 2022.01.28.478207 (2022).
10. Y Qian, et al., Programmable RNA sensing for cell monitoring and manipulation. bioRxiv. 2022.05.25.493141 (2022).
11. K Jiang, et al., Programmable eukaryotic protein expression with RNA sensors. bioRxiv, 2022.01.26.477951 (2022).
12. Y Song, et al., irCLASH reveals RNA substrates recognized by human ADARs. Nature Structural & Molecular Biology 27, 351-362 (2020).
13. K Licht, et al., Inosine induces context-dependent recoding and translational stalling. Nucleic Acids Research 47, 3 (2019).
14. M Uhlén, et al., Tissue-based map of the human proteome. Science 347, 1260419 (2015).
15. S Takyar, R P Hickerson, H F Noller, mRNA helicase activity of the ribosome. Cell 120, 49-58 (2005).
16. S Shao, R S Hegde, Membrane protein insertion at the endoplasmic reticulum. Annual Review of Cell and Developmental Biology 27, 25-56 (2011).
17. J E Wilusz, et al., A triple helix stabilizes the 3′ ends of long noncoding RNAs that lack poly(A) tails. Genes & Development 26, 2392-407 (2012).
18. H Poulsen, J Nilsson, C K Damgaard, J Egebjerg, J Kjems, CRM1 mediates the export of ADAR1 through a nuclear export signal within the Z-DNA dinding domain. Molecular and Cellular Biology 21, 7862-7871 (2001).
19. Z Wu, H Yang, P Colosi, Effect of genome size on AAV vector packaging. Molecular Therapy 18, 80-86 (2010).
20. D Katrekar, et al., In vivo RNA editing of point mutations via RNA-guided adenosine deaminases. Nature Methods 16, 239-242 (2019).
21. S Rauch, et al., Programmable RNA-guided RNA effector proteins built from human parts. Cell 178, 122-134 (2019).
22. J J Gam, B DiAndreth, R D Jones, J Huh, R Weiss, A ‘poly-transfection’ method for rapid, one-pot characterization and optimization of genetic systems. Nucleic Acids Research 47, e106-e106 (2019).
23. S Mustjoki, N S Young, Somatic mutations in “benign” disease. New England Journal of Medicine 384, 2039-2052 (2021).
24. A Poduri, G D Evrony, X Cal, C A Walsh, Somatic mutation, genomic variation, and neurological disease. Science 341, 1237758 (2013).
25. Catalogue Of Somatic Mutations In Cancer (COSMIC database) v95, Mutation ID: COSV52760651 (2021).
26. M R Bauer, et al., Targeting cavity-creating p53 cancer mutations with small-molecule stabilizers: the Y220X paradigm. ACS Chemical Biology 15, 657-668 (2020).
27. T Katagiri, et al., Bone morphogenetic protein-2 converts the differentiation pathway of C2C12 myoblasts into the osteoblast lineage. The Journal of Cell Biology 127, 1755-1766 (1994).
28. N M Angenent-Mari, A S Garruss, L R Soenksen, G Church, J J Collins, A deep learning approach to programmable RNA switches. Nature Communications 11, 1-12 (2020).
29. J A Valeri, et al., Sequence-to-function deep learning frameworks for engineered riboregulators. Nature Communications 11, 5058 (2020).
30. B Peters, M Nielsen, A Sette, T-cell epitope predictions. Annual Reviews in Immunology 38, 123-145 (2020).
31. T Gutschner, M Baas, S Diederichs, Non-coding RNA gene silencing through genomic integration of RNA destabilizing elements using zinc finger nucleases. Genome Research 21, gr.122358.111 (2011).
32. S Chen, Y Zhou, Y Chen, J Gu, fastp: an ultra-fast all-in-one FASTQ preprocessor. Bioinformatics 34, i884-i890 (2018).
33. A Dobin, et al., STAR: ultrafast universal RNA-seq aligner. Bioinformatics 29, 15-21 (2013).
34. E S Lander, et al., Initial sequencing and analysis of the human genome. Nature 409, 860-921 (2001).
35. A Frankish, et al., GENCODE reference annotation for the human and mouse genomes. Nucleic Acids Research 47, D766-D773 (2019).
36. C Lo Giudice, M A Tangaro, G Pesole, E Picardi, Investigating RNA editing in deep transcriptome datasets with REDItools and REDIportal. Nature Protocols 15, 1098-1131 (2020).
37. NT Swaidan, et al., Identification of potential transcription factors that enhance human iPSC generation. Scientific Reports 10, 21950 (2020).

### INCORPORATION BY REFERENCE

The present application refers to various issued patent, published patent applications, scientific journal articles, and other publications, all of which are incorporated herein by reference. The details of one or more embodiments of the invention are set forth herein. Other features, objects, and advantages of the invention will be apparent from the Detailed Description, the Figures, the Examples, and the Claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A nucleic acid comprising, from 5′ to 3′:
(i) a translation initiation sequence;
(ii) a sensor sequence comprising a premature stop codon;
(iii) a sequence encoding a base editor that acts on double stranded ribonucleic acid (dsRNA) and a sequence encoding an output, wherein the sequence encoding the base editor and the sequence encoding the output are in frame with the translation initiation sequence, optionally wherein the sequence encoding the base editor and the sequence encoding the output are separated by a sequence encoding a self-cleaving peptide;
optionally wherein the nucleic acid further comprises a sequence encoding a reporter that is in frame with the translation initiation sequence and which is 5′ to the premature stop codon;
optionally wherein (i) is separated from (ii) by a sequence encoding a self-cleaving peptide and/or (ii) is separated from (iii) by a sequence encoding a self-cleaving peptide.

2. The nucleic acid of claim 1, wherein the base editor comprises an adenosine deaminase acting on RNA (ADAR).

3. The nucleic acid of claim 1, wherein the output sequence comprises a therapeutic protein.

4. The nucleic acid of claim 3, wherein the therapeutic protein is capable of binding an RNA, optionally wherein the therapeutic protein comprises a guanine deaminase, a cytidine deaminase, an adenosine deaminase, or a uridine isomerase, optionally wherein the cytidine deaminase is an apolipoprotein B mRNA editing enzyme catalytic polypeptide (APOBEC), optionally wherein the therapeutic protein is capable of processing a gRNA into a mature component of a ribonucleoprotein (RNP).

5. (canceled)

6. The nucleic acid of claim 1, wherein the sensor sequence comprises an MS2 hairpin sequence, optionally wherein the sensor sequence comprises an MS2 hairpin sequence flanking each side of the premature stop codon, and wherein the base editor comprises an MS2 coat protein (MCP).

7. The nucleic acid of claim 1, wherein the premature stop codon is a TAG stop codon or a UAG stop codon, or wherein the sensor sequence comprises two or more premature stop codons, optionally wherein each of the two or more premature stop codons is a TAG stop codon or a UAG stop codon.

8.-9. (canceled)

10. The nucleic acid of claim 1, wherein the sensor sequence does not comprise an ATG start codon that: is positioned 3′ to a premature stop codon; and is in frame with the translation initiation sequence.

11. (canceled)

12. The nucleic acid of claim 1, wherein the sensor sequence does not comprise a TAG stop codon, a UAG stop codon, a TAA stop codon, a UAA stop codon, a TGA stop codon, or a UGA stop codon.

13. The nucleic acid of claim 1, wherein the nucleic acid comprises a sequence having at least 80%, at least 85%, at least 90%, at least 92%, at least 95%, at least 98%, or at least 99% identity to any one of SEQ ID NOs: 21-47, or wherein the nucleic acid comprises the sequence of any one of SEQ ID NOs: 21-47.

14. (canceled)

15. A vector or a recombinant viral genome comprising a nucleic acid sequence encoding the nucleic acid of claim 1.

16. (canceled)

17. A recombinant virus comprising
a nucleic acid encoding an RNA responsive sensor,
wherein the recombinant virus is a recombinant adeno-associated virus (rAAV) and the nucleic acid encoding the RNA-responsive sensor is flanked by a first and a second AAV inverted terminal repeat (ITR) or a recombinant lentivirus and the nucleic acid encoding the RNA-responsive sensor is flanked by a first and a second lentivirus long-inverted terminal repeat,
wherein the RNA-responsive sensor comprises:
(i) a translation initiation sequence;
(ii) a sensor sequence comprising a premature stop codon;
(iii) a sequence encoding a base editor that acts on double stranded ribonucleic acid (dsRNA) and a sequence encoding an output, wherein the sequence encoding the base editor and the sequence encoding the output are in frame with the translation initiation sequence, optionally wherein the sequence encoding the base editor and the sequence encoding the output are separated by a sequence encoding a self-cleaving peptide;
optionally wherein the nucleic acid further comprises a sequence encoding a reporter that is in frame with the translation initiation sequence and which is 5′ to the premature stop codon; optionally wherein (i) is separated from (ii) by a sequence encoding a self-cleaving peptide and/or (ii) is separated from (iii) by a sequence encoding a self-cleaving peptide.

18. (canceled)

19. A pharmaceutical composition comprising the nucleic acid of claim 1.

20. A genetic circuit comprising:
a) a nucleic acid encoding an RNA-responsive sensor comprising:
(i) a translation initiation sequence;
(ii) a sensor sequence comprising a premature stop codon;
(iii) a sequence encoding a base editor that acts on double stranded ribonucleic acid (dsRNA) and a sequence encoding an output, wherein the sequence encoding the base editor and the sequence encoding the output are in frame with the translation initiation sequence, optionally wherein the sequence encoding the base editor and the sequence encoding the output are separated by a sequence encoding a self-cleaving peptide;
optionally wherein the nucleic acid further comprises a sequence encoding a reporter that is in frame with the translation initiation sequence and which is 5′ to the premature stop codon; optionally wherein (i) is separated from (ii) by a sequence encoding a self-cleaving peptide and/or (ii) is separated from (iii) by a sequence encoding a self-cleaving peptide; and
b) a trigger nucleic acid comprising a trigger sequence, wherein the trigger sequence is capable of hybridizing with the sensor sequence of the RNA-responsive sensor, and wherein a nucleotide of the premature stop codon of the sensor sequence is non-complementary to the trigger sequence, and wherein the base editor of the RNA-responsive sensor is capable of editing the nucleotide of the premature stop codon of the sensor sequence that is non-complementary to the trigger sequence,
optionally wherein the genetic circuit comprises a vector or a recombinant viral genome comprising the nucleic acid sequence encoding the RNA-responsive sensor, optionally wherein the recombinant viral genome is a recombinant adeno-associated virus (AAV) genome.

21.-22. (canceled)

23. The genetic circuit of claim 20, wherein the trigger nucleic acid is a non-coding RNA or an mRNA, optionally wherein the region of the trigger sequence that is capable of hybridizing with the sensor sequence is within the 5′ UTR, the 3′ UTR, or an intron of the mRNA.

24.-25. (canceled)

26. The genetic circuit of claim 20, wherein the sensor sequence is designed to encode a TAG stop codon or a UAG stop codon at each position that aligns with a CCA site found in the trigger sequence.

27. The genetic circuit of claim 20, wherein fewer than 15 nucleic acid mismatches exist between the trigger sequence and the sensor sequence upon their hybridization, optionally wherein only one mismatch exists between the trigger sequence and the sensor sequence upon their hybridization.

28. (canceled)

29. A cell comprising the genetic circuit of claim 20 wherein the cell expresses an endogenous base editor that acts on dsRNA, optionally wherein the base editor comprises an ADAR.

30. A method of treating a disease, disorder, or condition in a subject comprising administering to the subject an RNA-responsive sensor comprising the nucleic acid of claim 1.

31.-33. (canceled)

34. A method of detecting an RNA molecule in a sample, comprising:
a) contacting the sample with the nucleic acid of claim 1, wherein the sample comprises: (i) a base editor that acts on double stranded RNA or a polynucleotide encoding the same; and (ii) mRNA translation machinery, optionally wherein the nucleic acid is introduced into a cell comprising (i) and (ii); and
b) detecting output that is produced.

35. (canceled)

36. A method of expressing a product of interest in a cell comprising introducing into the cell the nucleic acid of claim 1, wherein the output of the nucleic acid encodes the product of interest, and wherein the cell expresses: (i) a trigger nucleic acid comprising a trigger sequence, wherein the trigger sequence is capable of hybridizing with the sensor sequence of the nucleic acid, and wherein a nucleotide of the premature stop codon of the sensor sequence is non-complementary to the trigger sequence; and (ii) an endogenous base editor that is capable of editing the nucleotide of the premature stop codon of the sensor sequence that is non-complementary to the trigger sequence, optionally wherein the base editor comprises an ADAR.


SEQUENCE LISTING (77 sequences)

SEQ ID NO: 1 - RNA, 19 nt, Synthetic construct
  source 1..19: mol_type = other RNA
acatgaggatcacccatgt

SEQ ID NO: 2 - RNA, 19 nt, Synthetic construct
  source 1..19: mol_type = other RNA
acatgaggattacccatgt

SEQ ID NO: 3 - protein, 931 aa, Homo sapiens
MAEIKEKICDYLFNVSDSSALNLAKNIGLTKARDINAVLIDMERQGDVYRQGTTPPIWHLTDKKRERMQIKRNTNSVPETAPAAIPETKRNAEFLTCNIPTSNASNNMVTTEKVENGQEPVIKLENRQEARPEPARLKPPVHYNGPSKAGYVDFENGQWATDDIPDDLNSIRAAPGEFRAIMEMPSFYSHGLPRCSPYKKLTECQLKNPISGLLEYAQFASQTCEFNMIEQSGPPHEPRFKFQVVINGREFPPAEAGSKKVAKQDAAMKAMTILLEEAKAKDSGKSEESSHYSTEKESEKTAESQTPTPSATSFFSGKSPVTTLLECMHKLGNSCEFRLLSKEGPAHEPKFQYCVAVGAQTFPSVSAPSKKVAKQMAAEEAMKALHGEATNSMASDNQPEGMISESLDNLESMMPNKVRKIGELVRYLNTNPVGGLLEYARSHGFAAEFKLVDQSGPPHEPKFVYQAKVGGRWFPAVCAHSKKQGKQEAADAALRVLIGENEKAERMGFTEVTPVTGASLRRTMLLLSRSPEAQPKTLPLTGSTFHDQIAMLSHRCFNTLTNSFQPSLLGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPV

SEQ ID NO: 4 - protein, 1226 aa, Homo sapiens
MNPRQGYSLSGYYTHPFQGYEHRQLRYQQPGPGSSPSSFLLKQIEFLKGQLPEAPVIGKQTPSLPPSLPGLRPRFPVLLASSTRGRQVDIRGVPRGVHLGSQGLQRGFQHPSPRGRSLPQRGVDCLSSHFQELSIYQDQEQRILKFLEELGEGKATTAHDLSGKLGTPKKEINRVLYSLAKKGKLQKEAGTPPLWKIAVSTQAWNQHSGVVRPDGHSQGAPNSDPSLEPEDRNSTSVSEDLLEPFIAVSAQAWNQHSGVVRPDSHSQGSPNSDPGLEPEDSNSTSALEDPLEFLDMAEIKEKICDYLFNVSDSSALNLAKNIGLTKARDINAVLIDMERQGDVYRQGTTPPIWHLTDKKRERMQIKRNTNSVPETAPAAIPETKRNAEFLTCNIPTSNASNNMVTTEKVENGQEPVIKLENRQEARPEPARLKPPVHYNGPSKAGYVDFENGQWATDDIPDDLNSIRAAPGEFRAIMEMPSFYSHGLPRCSPYKKLTECQLKNPISGLLEYAQFASQTCEFNMIEQSGPPHEPRFKFQVVINGREFPPAEAGSKKVAKQDAAMKAMTILLEEAKAKDSGKSEESSHYSTEKESEKTAESQTPTPSATSFFSGKSPVTTLLECMHKLGNSCEFRLLSKEGPAHEPKFQYCVAVGAQTFPSVSAPSKKVAKQMAAEEAMKALHGEATNSMASDNQPEGMISESLDNLESMMPNKVRKIGELVRYLNTNPVGGLLEYARSHGFAAEFKLVDQSGPPHEPKFVYQAKVGGRWFPAVCAHSKKQGKQEAADAALRVLIGENEKAERMGFTEVTPVTGASLRRTMLLLSRSPEAQPKTLPLTGSTFHDQIAMLSHRCFNTLTNSFQPSLLGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPV

SEQ ID NO: 5 - protein, 741 aa, Homo sapiens
MDIEDEENMSSSSTDVKENRNLDNVSPKDGSTPGPGEGSQLSNGGGGGPGRKRPLEEGSNGHSKYRLKKRRKTPGPVLPKNALMQLNEIKPGLQYTLLSQTGPVHAPLFVMSVEVNGQVFEGSGPTKKKAKLHAAEKALRSFVQFPNASEAHLAMGRTLSVNTDFTSDQADFPDTLFNGFETPDKAEPPFYVGSNGDDSFSSSGDLSLSASPVPASLAQPPLPVLPPFPPPSGKNPVMILNELRPGLKYDFLSESGESHAKSFVMSVVVDGQFFEGSGRNKKLAKARAAQSALAAIFNLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEGSRSYTQAGVQWCNHGSLQPRPPGLLSDPSTSTFQGAGTTEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 6 - protein, 739 aa, Homo sapiens
MASVLGSGRGSGGLSSQLKCKSKRRRRRRSKRKDKVSILSTFLAPFKHLSPGITNTEDDDTLSTSSAEVKENRNVGNLAARPPPSGDRARGGAPGAKRKRPLEEGNGGHLCKLQLVWKKLSWSVAPKNALVQLHELRPGLQYRTVSQTGPVHAPVFAVAVEVNGLTFEGTGPTKKKAKMRAAELALRSFVQFPNACQAHLAMGGGPGPGTDFTSDQADFPDTLFQEFEPPAPRPGLAGGRPGDAALLSAAYGRRRLLCRALDLVGPTPATPAAPGERNPVVLLNRLRAGLRYVCLAEPAERRARSFVMAVSVDGRTFEGSGRSKKLARGQAAQAALQELFDIQMPGHAPGRARRTPMPQEFADSISQLVTQKFREVTTDLTPMHARHKALAGIVMTKGLDARQAQVVALSSGTKCISGEHLSDQGLVVNDCHAEVVARRAFLHFLYTQLELHLSKRREDSERSIFVRLKEGGYRLRENILFHLYVSTSPCGDARLHSPYEITTDLHSSKHLVRKFRGHLRTKIESGEGTVPVRGPSAVQTWDGVLLGEQLITMSCTDKIARWNVLGLQGALLSHFVEPVYLQSIVVGSLHHTGHLARVMSHRMEGVGQLPASYRHNRPLLSGVSDAEARQPGKSPPFSMNWVVGSADLEIINATTGRRSCGGPSRLCKHVLSARWARLYGRLSTRTPSPGDTPSMYCEAKLGAHTYQSVKQQLFKAFQKAGLGTWVRKPPEQQQFLLTL

SEQ ID NO: 7 - protein, 117 aa, Synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 8 - protein, 386 aa, Synthetic construct
MQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 9 - protein, 541 aa, Synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSGSGSLPPLERLTL

SEQ ID NO: 10 - protein, 19 aa, Synthetic construct
ATNFSLLKQAGDVEENPGP

SEQ ID NO: 11 - protein, 18 aa, Synthetic construct
EGRGSLLTCGDVEENPGP

SEQ ID NO: 12 - protein, 20 aa, Synthetic construct
QCTNYALLKLAGDVESNPGP

SEQ ID NO: 13 - protein, 230 aa, Synthetic construct
MSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGH

SEQ ID NO: 14 - protein, 236 aa, Synthetic construct
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYK

SEQ ID NO: 15 - protein, 171 aa, Synthetic construct
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 16 - DNA, 951 bp, Synthetic construct
  source 1..951: mol_type = other DNA
atggcggaaggatccgtcgccaggcagcctgacctcttgacctgcgacgatgagccgatccatatccccggtgccatccaaccgcatggactgctgctcgccctcgccgccgacatgacgatcgttgccggcagcgacaaccttcccgaactcaccggactggcgatcggcgccctgatcggccgctctgcggccgatgtcttcgactcggagacgcacaaccgtctgacgatcgccttggccgagcccggggcggccgtcggagcaccgatcactgtcggcttcacgatgcgaaaggacgcaggcttcatcggctcctggcatcgccatgatcagctcatcttcctcgagctcgagcctccccagcgggacgtcgccgagccgcaggcgttcttccgccgcaccaacagcgccatccgccgcctgcaggccgccgaaaccttggaaagcgcctgcgccgccgcggcgcaagaggtgcggaagattaccggcttcgatcgggtgatgatctatcgcttcgcctccgacttcagcgggtccgtgatcgcagaggatcggtgcgccgaggtcgagtcaaaactaggcctgcactatcctgcctcattcatcccggcgcaggcccgtcggctctataccatcaacccggtacggatcattcccgatatcaattatcggccggtgccggtcaccccagacctcaatccggtcaccgggcggccgattgatcttagcttcgccatcctgcgcagcgtctcgcccaaccatctggagttcatgcgcaacataggcatgcacggcacgatgtcgatctcgattttgcgcggcgagcgactgtggggattgatcgtttgccatcaccgaacgccgtactacgtcgatctcgatggccgccaagcctgcgagctagtcgcccaggttctggcctggcagatcggcgtgatggaagagtaa

SEQ ID NO: 17 - DNA, 950 bp, Synthetic construct
  source 1..950: mol_type = other DNA
atgcggaaggatccgtcgccaggcagcctgacctcttgacctgcgacgatgagccgatccatatccccggtgccatccaaccgcatggactgctgctcgccctcgccgccgacatgacgatcgttgccggcagcgacaaccttcccgaactcaccggactggcgatcggcgccctgatcggccgctctgcggccgatgtcttcgactcggagacgcacaaccgtctgacgatcgccttggccgagcccggggcggccgtcggagcaccgatcactgtcggcttcacgatgcgaaaggacgcaggcttcatcggctcctggcatcgccatgatcagctcatcttcctcgagctcgagcctccccagcgggacgtcgccgagccgcaggcgttcttccgccgcaccaacagcgccatccgccgcctgcaggccgccgaaaccttggaaagcgcctgcgccgccgcggcgcaagaggtgcggaagattaccggcttcgatcgggtgatgatctatcgcttcgcctccgacttcagcgggtccgtgatcgcagaggatcggtgcgccgaggtcgagtcaaaactaggcctgcactatcctgcctcattcatcccggcgcaggcccgtcggctctataccatcaacccggtacggatcattcccgatatcaattatcggccggtgccggtcaccccagacctcaatccggtcaccgggcggccgattgatcttagcttcgccatcctgcgcagcgtctcgcccaaccatctggagttcatgcgcaacataggcatgcacggcacgatgtcgatctcgattttgcgcggcgagcgactgtggggattgatcgtttgccatcaccgaacgccgtactacgtcgatctcgatggccgccaagcctgcgagctagtcgcccaggttctggcctggcagatcggcgtgatggaagagtaa

SEQ ID NO: 18 - DNA, 1038 bp, Synthetic construct
  source 1..1038: mol_type = other DNA
atgggcgtgaaggtcctcttcgcactcatttgcatagccgtagcagaagccgactataaagacgacgatgacaagggcgggtccggcggagcggaaggatccgtcgccaggcagcctgacctcttgacctgcgacgatgagccgatccatatccccggtgccatccaaccgcatggactgctgctcgccctcgccgccgacatgacgatcgttgccggcagcgacaaccttcccgaactcaccggactggcgatcggcgccctgatcggccgctctgcggccgatgtcttcgactcggagacgcacaaccgtctgacgatcgccttggccgagcccggggcggccgtcggagcaccgatcactgtcggcttcacgatgcgaaaggacgcaggcttcatcggctcctggcatcgccatgatcagctcatcttcctcgagctcgagcctccccagcgggacgtcgccgagccgcaggcgttcttccgccgcaccaacagcgccatccgccgcctgcaggccgccgaaaccttggaaagcgcctgcgccgccgcggcgcaagaggtgcggaagattaccggcttcgatcgggtgatgatctatcgcttcgcctccgacttcagcgggtccgtgatcgcagaggatcggtgcgccgaggtcgagtcaaaactaggcctgcactatcctgcctcattcatcccggcgcaggcccgtcggctctataccatcaacccggtacggatcattcccgatatcaattatcggccggtgccggtcaccccagacctcaatccggtcaccgggcggccgattgatcttagcttcgccatcctgcgcagcgtctcgcccaaccatctggagttcatgcgcaacataggcatgcacggcacgatgtcgatctcgattttgcgcggcgagcgactgtggggattgatcgtttgccatcaccgaacgccgtactacgtcgatctcgatggccgccaagcctgcgagctagtcgcccaggttctggcctggcagatcggcgtgatggaagagtaa

SEQ ID NO: 19 - DNA, 1182 bp, Homo sapiens
  source 1..1182: mol_type = genomic DNA
atggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccgcgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactag

SEQ ID NO: 20 - DNA, 1182 bp, Homo sapiens
  source 1..1182: mol_type = genomic DNA
atggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccgcgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccccatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactag

SEQ ID NO: 21 - DNA, 75 bp, Synthetic construct
  source 1..75: mol_type = other DNA
agcagcagtccatgcggttggattgcaccggggatatagatcggctcatcgtcgcaggtcaagaggtcaggctgc

SEQ ID NO: 22 - DNA, 75 bp, Synthetic construct
  source 1..75: mol_type = other DNA
agcagcagtccatgcggttagattgcaccggggatatagatcggctcatcgtcgcaggtcaagaggtcaggctgc

SEQ ID NO: 23 - DNA, 75 bp, Synthetic construct
  source 1..75: mol_type = other DNA
cggcggcgagggcgagcagcagtccatgcggttggatagcaccggggatatggatcggctcatcgtcgcaggtca

SEQ ID NO: 24 - DNA, 75 bp, Synthetic construct
  source 1..75: mol_type = other DNA
atgtcggcggcgagggcgagcagcagtccatgcggttagattgcaccggggatatggatcggctcatcgtcgcag

SEQ ID NO: 25 - DNA, 75 bp, Synthetic construct
  source 1..75: mol_type = other DNA
atgtcggcggcgagggcgagcagcagtccatgcggttagattgcaccggggatatagatcggctcatcgtcgcag

SEQ ID NO: 26 - DNA, 75 bp, Synthetic construct
  source 1..75: mol_type = other DNA
cgctttccaaggtttcggcggcctgcaggcggcggatagcgctgttggtgcggcggaagaacgcctgcggctcgg

SEQ ID NO: 27 - DNA, 75 bp, Synthetic construct
  source 1..75: mol_type = other DNA
cgctttccaaggtttcggcggcctgcaggcggcggatagcgctgttagtgcggcggaagaacgcctgcggctcgg

SEQ ID NO: 28 - DNA, 75 bp, Synthetic construct
  source 1..75: mol_type = other DNA
gtgccgtgcattcctattttgcgcattaactccagatagttgggcgagacgctgcgcaggattgcgaagctaaga

SEQ ID NO: 29 - DNA, 123 bp, Synthetic construct
  source 1..123: mol_type = other DNA
gcggcgagggcgagcagacatgaggatcacccatgttgcggttggattgcaccggggatatagatcggctcatcgtcgcaggtcaaacatgaggatcacccatgtggctgcctggcgacggat

SEQ ID NO: 30 - DNA, 123 bp, Synthetic construct
  source 1..123: mol_type = other DNA
gcggcgagggcgagacatgaggatcacccatgtccatgcggttggattgcaccggggatatagatcggctcatcgtcgcaggtacatgaggatcacccatgttcaggctgcctggcgacggat

SEQ ID NO: 31 - DNA, 123 bp, Synthetic construct
  source 1..123: mol_type = other DNA
gcggcgagggcgagcagcagacatgaggatcacccatgtggttggattgcaccggggatatagatcggctcatcgtcgcaggtcaagagacatgaggatcacccatgttgcctggcgacggat

SEQ ID NO: 32 - DNA, 123 bp, Synthetic construct
  source 1..123: mol_type = other DNA
cgatcgtcatgtcggcgacatgaggatcacccatgtcgagcagcagtccatgcggttggatagcaccggggatatggatcggctcaacatgaggatcacccatgtaggtcaagaggtcaggct

SEQ ID NO: 33 - DNA, 123 bp, Synthetic construct
  source 1..123: mol_type = other DNA
cgatcgtcatgtcgacatgaggatcacccatgtgggcgagcagcagtccatgcggttggatagcaccggggatatggatcggcacatgaggatcacccatgtcgcaggtcaagaggtcaggct

SEQ ID NO: 34 - DNA, 123 bp, Synthetic construct
  source 1..123: mol_type = other DNA
cgatcgtcatgtcggcggcgacatgaggatcacccatgtgcagcagtccatgcggttggatagcaccggggatatggatcggctcatcgacatgaggatcacccatgttcaagaggtcaggct

SEQ ID NO: 35 - DNA, 123 bp, Synthetic construct
  source 1..123: mol_type = other DNA
gcaacgatcgtcatttcacatgaggatcacccatgtagggcgagcagcagtccatgcggttagattgcaccggggatatggatcggacatgaggatcacccatgttcgcaggtcaagaggtca

SEQ ID NO: 36 - DNA, 123 bp, Synthetic construct
  source 1..123: mol_type = other DNA
gcaacgatcgtcatacatgaggatcacccatgtgcgagggcgagcagcagtccatgcggttagattgcaccggggatatggatacatgaggatcacccatgttcgtcgcaggtcaagaggtca

SEQ ID NO: 37 - DNA, 123 bp, Synthetic construct
  source 1..123: mol_type = other DNA
gcaacgatcgtcatttcggcacatgaggatcacccatgtgcgagcagcagtccatgcggttagattgcaccggggatatggatcggctcacatgaggatcacccatgtcaggtcaagaggtca

SEQ ID NO: 38 - DNA, 123 bp, Synthetic construct
  source 1..123: mol_type = other DNA
cagttgcagtggattgtacatgaggatcacccatgttcagagccaacctcaggcggctcatagggcaccaccacactatgtcgaaaacatgaggatcacccatgtctgtcatccaaatactcc

SEQ ID NO: 39 - DNA, 123 bp, Synthetic construct
  source 1..123: mol_type = other DNA
ccacttaaaagccccctacatgaggatcacccatgtaagggatggcttttgacaccaacttaggggctcacatgcacacccagcctacatgaggatcacccatgtaatctcagttgggcatgg

SEQ ID NO: 40 - DNA, 123 bp, Synthetic construct
  source 1..123: mol_type = other DNA
gaagctcctgagtttgcacatgaggatcacccatgtagggacattaacaagggggctctctagactccatctttctctcctcagaaacatgaggatcacccatgtctctctgctttaaggagt

SEQ ID NO: 41 - DNA, 123 bp, Synthetic construct
  source 1..123: mol_type = other DNA
agctcctcccccttctcacatgaggatcacccatgttcagggcaggcccagcccagccactagcttcaggaagagactagaacagaacatgaggatcacccatgtacttgtccaggtcagggc

SEQ ID NO: 42 - DNA, 123 bp, Synthetic construct
  source 1..123: mol_type = other DNA
ggcagctttacaaacaaacatgaggatcacccatgtaacaataaacaatacacaaagcactagaaggttcccaagatccactgcaaacatgaggatcacccatgtgcccccagaggctttgga

SEQ ID NO: 43 - DNA, 123 bp, Synthetic construct
  source 1..123: mol_type = other DNA
cccacccccttccctgcacatgaggatcacccatgtcggtatcatcagcacaggagaccttagtcagacggcagctttacaaacaaacatgaggatcacccatgtaacaataaacaatacaca

SEQ ID NO: 44 - DNA, 123 bp, Synthetic construct
  source 1..123: mol_type = other DNA
aaaggaaatccaaataaacatgaggatcacccatgtaaagaattacaaaagaaaaaaaattagcaaaaccacacaattcttagttcacatgaggatcacccatgtaagtcaccccaagagccc

SEQ ID NO: 45 - DNA, 123 bp, Synthetic construct
  source 1..123: mol_type = other DNA
acaaaggggaatttgtcacatgaggatcacccatgtagccgggtctcctcgcccgtgttgtagtgtagctggcccttaaggattcgacatgaggatcacccatgtgttactgtggagacgccc

SEQ ID NO: 46 - DNA, 123 bp, Synthetic construct
  source 1..123: mol_type = other DNA
tggattggacctcatggacatgaggatcacccatgttggtgttgcatcgcgtgcgctctgtagctgcgctcactcccactgtgcccacatgaggatcacccatgtccttcacgccacacaagt

SEQ ID NO: 47 - DNA, 123 bp, Synthetic construct
  source 1..123: mol_type = other DNA
agctcttccaaatacggacatgaggatcacccatgtccaggccatctagccttgtacccctagccttctcatccagttcgtattccacatgaggatcacccatgtttctgttcttcgggtaca

SEQ ID NO: 48 - DNA, 24 bp, Synthetic construct
  source 1..24: mol_type = other DNA
ccggaggccctagatcttcttgac

SEQ ID NO: 49 - DNA, 20 bp, Synthetic construct
  source 1..20: mol_type = other DNA
gggactgctccttcaccacc

SEQ ID NO: 50 - DNA, 21 bp, Synthetic construct
  source 1..21: mol_type = other DNA
ttgctcagctccctcaaccag

SEQ ID NO: 51 - DNA, 20 bp, Synthetic construct
  source 1..20: mol_type = other DNA
agccgcgagcaaatgatctc

SEQ ID NO: 52 - DNA, 20 bp, Synthetic construct
  source 1..20: mol_type = other DNA
ggcgcatcaaggagctcacc

SEQ ID NO: 53 - DNA, 19 bp, Synthetic construct
  source 1..19: mol_type = other DNA
cctgctcctccgcctcctc

SEQ ID NO: 54 - DNA, 25 bp, Synthetic construct
  source 1..25: mol_type = other DNA
cacctgccttaccaactcttttgtg

SEQ ID NO: 55 - DNA, 25 bp, Synthetic construct
  source 1..25: mol_type = other DNA
ggctacattggtgttgagcttttgg

SEQ ID NO: 56 - DNA, 18 bp, Synthetic construct
  source 1..18: mol_type = other DNA
ttaagggcctgcagggtg

SEQ ID NO: 57 - DNA, 17 bp, Synthetic construct
  source 1..17: mol_type = other DNA
tggctagcccctcgagt

SEQ ID NO: 58 - DNA, 23 bp, Synthetic construct
  source 1..23: mol_type = other DNA
ctgaaacaggcaggagatgtgga

SEQ ID NO: 59 - DNA, 23 bp, Synthetic construct
  source 1..23: mol_type = other DNA
ccgcatgtaagcagacttcctct

SEQ ID NO: 60 - DNA, 22 bp, Synthetic construct
  source 1..22: mol_type = other DNA
tgctctcctgttgtgcttctcc

SEQ ID NO: 61 - DNA, 21 bp, Synthetic construct
  source 1..21: mol_type = other DNA
agcctcccattcaattgccac

SEQ ID NO: 62 - DNA, 26 bp, Synthetic construct
  source 1..26: mol_type = other DNA
aagaagaaaaaagcatctgagcctgg

SEQ ID NO: 63 - DNA, 21 bp, Synthetic construct
  source 1..21: mol_type = other DNA
agcctcccattcaattgccac

SEQ ID NO: 64 - DNA, 25 bp, Synthetic construct
  source 1..25: mol_type = other DNA
acgaccactttgtcaagctcatttc

SEQ ID NO: 65 - DNA, 25 bp, Synthetic construct
  source 1..25: mol_type = other DNA
gcagtgagggtctctctcttcctct

SEQ ID NO: 66 - protein, 10 aa, Synthetic construct
GCCRCSATGN

SEQ ID NO: 67 - DNA, 10 bp, Synthetic construct
  source 1..10: mol_type = other DNA
gccaccatga

SEQ ID NO: 68 - protein, 8 aa, Synthetic construct
DXEXNPGP

SEQ ID NO: 69 - DNA, 29 bp, synthetic construct
  source 1..29: mol_type = other DNA
  modified_base 15: mod_base = OTHER; note = May be replaced with guanosine
tgcaccggggatatagatcggctcatcgt

SEQ ID NO: 70 - DNA, 99 bp, synthetic construct
  source 1..99: mol_type = other DNA
atccgtcgccaggcagcctgacctcttgacctgcgacgatgagccgatccatatccccggtgccatccaaccgcatggactgctgctcgccctcgccgc

SEQ ID NO: 71 - DNA, 100 bp, synthetic construct
  source 1..100: mol_type = other DNA
gcggcgagggcgagcagcagtccatgcggttggatggcaccggggatatggatcggctcatcgtcgcaggtcaagaggtcaggctgcctggcgacgggat

SEQ ID NO: 72 - DNA, 21 bp, synthetic construct
  source 1..21: mol_type = other DNA
attaagggcctgcagggtgga

SEQ ID NO: 73 - DNA, 21 bp, synthetic construct
  source 1..21: mol_type = other DNA
tctactcgaggggctagccag

SEQ ID NO: 74 - DNA, 101 bp, synthetic construct
  source 1..101: mol_type = other DNA
gcggcgaggggcgagcagcagtccatgcggttggatggcaccggggatatagatcggctcatcgtcgcaggtcaaggaggtcaggctgcctggcgacggat

SEQ ID NO: 75 - DNA, 19 bp, synthetic construct
  source 1..19: mol_type = other DNA
acatgaggatcacccatgt

SEQ ID NO: 76 - DNA, 63 bp, synthetic construct
  source 1..63: mol_type = other DNA
gcggcgagggcgagcagacatgaggatcacccatgttgcggttggatggcaccggggatatag

SEQ ID NO: 77 - DNA, 60 bp, synthetic construct
  source 1..60: mol_type = other DNA
atcggctcatcgtcgcaggtcaaacatgaggatcacccatgtggctgcctggcgacggat